SEQUENCE LISTING (11 sequences)

SEQ ID NO: 1 - DNA, 25 bp, Artificial Sequence
  Synthetic- primer
agggagtcaacgatcgcacgggagg

SEQ ID NO: 2 - DNA, 25 bp, Artificial Sequence
  Synthetic- primer
cacagacagggagtcttagccaggg

SEQ ID NO: 3 - DNA, 22 bp, Artificial Sequence
  Synthetic- primer
ctgtgtcttctggccaagtgtt

SEQ ID NO: 4 - DNA, 19 bp, Artificial Sequence
  Synthetic- primer
tcggttctcagagactagc

SEQ ID NO: 5 - DNA, 23 bp, Artificial Sequence
  Synthetic- primer
gcggtctggcagtaaaaactatc

SEQ ID NO: 6 - DNA, 23 bp, Artificial Sequence
  Synthetic- primer
gtgaaacagcattgctgtcactt

SEQ ID NO: 7 - DNA, 20 bp, Artificial Sequence
  Synthetic- primer
ggagggagtcaacgatcgca

SEQ ID NO: 8 - DNA, 20 bp, Artificial Sequence
  Synthetic- primer
aatgggggacctactcacca

SEQ ID NO: 9 - DNA, 19 bp, Artificial Sequence
  Synthetic- primer
gcactcatccaggctgtcc

SEQ ID NO: 10 - DNA, 20 bp, Artificial Sequence
  Synthetic- primer
ataccggagatcatgcaagc

SEQ ID NO: 11 - DNA, 20 bp, Artificial Sequence
  Synthetic- primer
aggtggacctgatcatggag

(19) United States
(12) Patent Application Publication
(10) Pub. No.: US 2022/0175738 A1
(43) Pub. Date: Jun. 9, 2022

(54) Tip60 Inhibitors and Methods of Use for Cardiovascular Disease

(71) Applicant: The Medical College of Wisconsin, Inc., Milwaukee, WI (US)

(72) Inventors: John William Lough, Elm Grove, WI (US); John Allen Auchampach, Elm Grove, WI (US)

(21) Appl. No.: 17/442,968

(22) PCT Filed: Mar. 31, 2020
(86) PCT No.: PCT/US2020/025923

§ 371 Date: Sep. 24, 2021

Related U.S. Application Data
(60) Provisional application No. 62/827,519, filed on Apr. 1, 2019.

(51) Int. Cl. A61K 31/427; A61K 45/06; A61P 9/04

(52) U.S. Cl. CPC A61K 31/427; A61P 9/04; A61K 45/06

(57) ABSTRACT

The present invention relates to compositions and methods of inducing cardiomyocyte proliferation by transiently contacting the cardiomyocytes with a Tip60 inhibitor. The present invention also provides methods of treating cardiac injury, myocardial infarction and methods of regenerating cardiac tissue.

### CROSS-REFERENCE TO RELATED APPLICATIONS

This patent application claims the benefit of priority of U.S. Provisional Patent Application No. 62/827,519, filed Apr. 1, 2019, which is incorporated herein by reference in its entirety.

### STATEMENT REGARDING FEDERALLY SPONSORED RESEARCH

This invention was made with government under Grant Numbers 5R01HL131788 and 1S10 OD025038, both awarded by the National Institutes of Health. The government has certain rights in this invention.

### SEQUENCE LISTING

A Sequence Listing accompanies this application and is submitted as an ASCII text file of the sequence listing named “650053_00682_ST25.txt” which is 2.34 KB in size and was created on Mar. 18, 2020. The sequence listing is electronically submitted via EFS-Web with the application and is incorporated herein by reference in its entirety.

### INTRODUCTION

Cardiac diseases and injury are conditions that result in cardiac cell injury and reduced cardiac function. Heart attack, or myocardial infarction (MI), is a leading cause of cardiac injury and is one of the leading causes of death for men and women in the US and in developed countries. Myocardial infarction is the irreversible death (i.e. necrosis) of heart muscle, which is caused by ischemia due to prolonged lack of oxygen supply. Approximately 1.5 million cases of MI occur annually in the United States.

The prevalence of cardiac diseases has steadily increased in the population of the western developed countries over the last few years. One reason for said increase can be seen in an increased average life expectation and improved survival after myocardial infarction due to modern medicine. The mortality rate caused by cardiac disease, however, could be further reduced by novel therapeutic approaches to regenerate damaged heart tissue and thus improve recovery rates from cardiac diseases and injury.

The pathogenesis of MI is largely attributed to the loss of cardiomyocytes (CMs) and their insufficient regeneration. Inducing the proliferation of pre-existing CMs has emerged as a potential therapeutic strategy for cardiac repair. There is a need for drugs, methods and compositions for increasing proliferation of CMs in response to MI.

### SUMMARY

In one aspect, the present invention provides methods of inducing proliferation of adult cardiomyocytes (CMs). The methods comprise transiently contacting the adult CMs with an effective amount of a Tip60 inhibitor to induce proliferation of cardiomyocytes.

In a second aspect, the present invention provides methods of regenerating heart tissue within a patient in need thereof. The methods comprise administering an effective amount of a Tip60 inhibitor to transiently induce proliferation and regeneration of heart tissue.

In a third aspect, the present invention provides methods of treating a subject with cardiac injury. The methods comprise administering a therapeutically effective amount of a Tip60 inhibitor to treat the cardiac injury.

In a final aspect, the present invention provides medicaments for use in a method of proliferating cardiac cells or cardiomyocytes in a patient, the method comprising administering an effective amount of Tip60 inhibitor to cause cardiomyocytes to proliferate.

### BRIEF DESCRIPTION OF THE DRAWINGS

FIG. 1 shows that tamoxifen activation of Cre-recombinase at P0 causes Tip60 depletion in the heart at successive postnatal stages. Control (Kat5^{f/f}) and experimental (Kat5^{f/f;Myh6-merCremer}, denoted Kat5^{Δ/Δ}) pups were injected with 250 μg tamoxifen on the day of birth to initiate recombination of the floxed Kat5 gene. Panel a schematically displays how the control and knockout genotypes are designated in this paper, and the postnatal days when hearts were harvested (H) for analysis. Panel b shows results from qRT/PCR analyses revealing extent of Kat5 mRNA knockdown at each neonatal day. Panel c is a western blot showing Tip60 protein depletion at P12. Error bars denote ±SEM. Statistical significance was determined using an unpaired two-tailed t-test.

FIG. 2 shows that levels of phospho-Atm (pAtm) and bulk Atm in the heart are increased early in the neonatal period. Protein was isolated from wild-type mouse hearts at the indicated stages of development (E11-P31); sampling the earliest embryonic stages required pooling of hearts. Proteins were separated on a 4-15% gradient acrylamide/SDS gels, followed by electro-blotting onto nitrocellulose and reacting with antibodies to detect pAtm (panel a) and bulk Atm (panel b). Atm and pAtm levels were normalized to equivalent total protein (10 μg/lane; Ponceau S-stained gel, panel c) instead of Gapdh due to changing levels of the latter during the glycolytic-aerobic transition at the time of birth. Because Atm migrates at ˜350 kid, panels a and b only show proteins >100 kD. E=embryonic day; P=postnatal day

FIG. 3 shows that pAtm-positive CMs are decreased in Tip60-depleted hearts. Hearts in control (Kat5^{f/f}) and experimental (Kat^{Δ/Δ}) neonates that were administered tamoxifen at P0 were harvested on the indicated postnatal days, followed by double immunostaining to co-detect GATA4, as indicative of CM identity, and pAtm. Panels a-f show typical staining patterns in a P12 heart. Panel g displays percentages of pAtm-positive CMs enumerated by blinded observers while scanning entire sections at 1000× magnification. Vertical lines denote ±SEM. [Ns: P7, 3 v 4; P12, 7 v 8; P39, 7 v 7]

FIG. 4 shows that decreased expression of cell cycle inhibitors Meis1 and p27 in Tip60-depleted hearts. Following administration of tamoxifen at neonatal day P0, hearts were removed from control (Kat5^{f/f}) and experimental (Kat^{Δ/Δ}) mice on the indicated postnatal days and subjected to qRT-PCR using the Taqman probes listed in Table 1. In order to use normalizers with optimally stable expression, Rpl37α and Gapdh were employed P7/P12 and P39, respectively. Vertical lines denote ±SEM. [Ns: P7, 3 v 4; P12, 6 v 9; P39, 6 v 7]

FIG. 5 shows that activation of the cardiomyocyte cell cycle in Tip60-depleted CMs. Following injection of tamoxifen at P0, hearts harvested on postnatal days P7, P12 and P39 were immunostained for Ki67 (panel a), BrdU (panel b), and phosphohistone H3 (pH3) (panel c), plus GATA4 to identify CMs. Percentages of cell cycle-activated CMs were enumerated by blinded observers while scanning entire sections at 1000× magnification. Vertical lines indicate ±SEM. Bar graphs displaying individual data points are shown in FIG. 16.

FIG. 6 shows that Tip60 depletion increases the percentage of mononuclear diploid cardiomyocytes at P12. Ventricles of Kat5^{f/f} (control) and Kat5^{Δ/Δ} (Tip60-depleted) neonatal mice injected with 250 μg tamoxifen at P0 were dis-aggregated into single cells at P12, followed by immunofluorescent staining with cTnT and DAPI. In panel a, a minimum of 200 cTnT-positive CMs per heart were evaluated to discern mono- vs. multi-nucleation as revealed by numbers of DAPI-positive nuclei/cell. In panel b, DAPI-positive nuclei were assessed for relative ploidy by determining pixilation density using ImageJ analysis. Vertical lines indicate ±SEM. Statistical significance was determined using unpaired two-tailed t-tests.

FIG. 7 shows that Tip60 depletion may improve cardiac function and reduce scarring after myocardial infarction (MI). Kat5^{f/f} and Kat5^{Δ/Δ} hearts that had been treated with tamoxifen on P0 were infarcted on P7 via permanent ligation of the left main coronary artery. On P39, cardiac function was assessed by echocardiography (panels c-e), after which the hearts were harvested and transverse sections removed at equal intervals below the ventricular midline were processed to evaluate scarring by Masson trichrome staining (panels a-b), which was quantitated by area and midline length. Statistical significance was determined using unpaired two-tailed t-tests.

FIG. 8 shows gross anatomical parameters of Tip60-depleted hearts. Kat5^{f/f} and Kat5^{Δ/Δ} hearts (left column), and Kat and Kat^{+/+} and Kat^{+/+;Myh6-merCremer} mice (right column), were treated with 250 μg tamoxifen on neonatal day P0. On the indicated days (panel a: P7, panel b: P12, panel c: P39), the animals were weighed to obtain total body weight (BW; grams), followed by harvesting and weighing hearts (HW; mg). Vertical lines indicate ±SEM. Statistical significance was determined using unpaired two-tailed t-tests.

FIG. 9 shows the absence of scarring in Tip60-depleted neonatal hearts. Kat5^{f/f} and Kat5^{Δ/Δ} hearts were treated with tamoxifen on P0 and harvested on the indicated days, followed by histologic processing to assess scarring via Masson trichrome staining. Scanning sections of each heart at 1,000× revealed no evidence of scarring.

FIG. 10 shows the results of TUNEL histochemistry, which demonstrate that cell death is not altered in hearts containing Tip60-depleted CMs. Kat5^{f/f} and Kat5^{Δ/Δ} mice were injected with 250 μg tamoxifen at P0. Hearts were harvested for histology on postnatal days P7 (panel a), P12 (panel b), and P39 (panel c). Sections were reacted to detect fragmented DNA using TUNEL reagents, followed by microscopically scanning, in blind, the entirety of each section at 1,000× to determine the number of TUNEL-positive cells. Vertical lines indicate ±SEM. P-values were determined using unpaired two-tailed t-tests.

FIG. 11 shows that Tip60 depletion in CMs beginning on P0 alters expression of cell cycle activation genes on subsequent postnatal days. Following administration of tamoxifen at neonatal day P0, hearts were removed from control (Kat5^{f/f}) and experimental (Kat^{Δ/Δ}) mice at days P7, P12, and P39, RNA was purified and reverse-transcribed, and cDNAs encoded by the indicated target genes were amplified in triplicate by quantitative PCR using the Taqman probe kits listed in Table 1. In order to use normalizers with optimally stable expression, Rpl37α and Gapdh were employed P7/P12 and P39, respectively. G1 cell cycle activators are shown in panel a, and G2 cell cycle activators are shown in panel b. Vertical lines denote ±SEM. [Ns: P7, 3 v 4; P12, 6 v 9; P39, 6 v 7]

FIG. 12 shows the results of qRT-PCR assays, which demonstrate that Tip60 depletion in CMs beginning on P0 causes increased expression of the de-differentiation marker Myh7 on subsequent postnatal days. To use normalizers with optimally stable expression, Rpl37α and Gapdh were employed at P7/P12 and P39, respectively. Vertical lines denote ±SEM. [Ns: P7, 3 v 4; P12, 6 v 9; P39, 6 v 7]

FIG. 13 shows the results of qRT-PCR assays, which demonstrate that Cre-recombinase, alone, did not alter expression of cell cycle target genes at neonatal stage P9. Following administration of tamoxifen at neonatal day P0 to wild-type (Kat5^{+/+}) mice and to littermates expressing the merCremer-recombinase transgene (Kat5^{+/+;merCremer}) hearts were removed at postnatal day 9. RNA was purified and reverse-transcribed, and cDNAs encoded by the indicated target genes were amplified in triplicate by quantitative PCR using the Taqman probe kits listed in Table 1. Cell cycle activators are shown in panel a, and cell cycle inhibitors and de-differentiation markers are shown in panel b.

FIG. 14 shows the results of qRT-PCR assays, which demonstrate that Cre-recombinase, alone, did not alter expression of cell cycle target genes at neonatal stage P12. Following administration of tamoxifen at neonatal day P0 to wild-type (Kat5^{+/+}) mice and to littermates expressing the merCremer-recombinase transgene (Kat5^{+/+;merCremer}), hearts were removed at postnatal day 12. RNA was purified and reverse-transcribed, and cDNAs encoded by the indicated target genes were amplified in triplicate by quantitative PCR using the Taqman probe kits listed in Table 1. Cell cycle activators are shown in panel a, and cell cycle inhibitors and de-differentiation markers are shown in panel b.

FIG. 15 shows the results of qRT-PCR assays, which demonstrate that Cre-recombinase, alone, did not alter expression of cell cycle target genes at neonatal stage P41. Following administration of tamoxifen at neonatal day P0 to wild-type (Kat5^{+/+}) mice and to littermates expressing the merCremer-recombinase transgene (Kat5^{+/+;merCremer}), hearts were removed at postnatal day 41. RNA was purified and reverse-transcribed, and cDNAs encoded by the indicated target genes were amplified in triplicate by quantitative PCR using the Taqman probe kits listed in Table 1. Cell cycle activators are shown in panel a, and cell cycle inhibitors and de-differentiation markers are shown in panel b.

FIG. 16 shows the activation of the cardiomyocyte cell cycle in Tip60-depleted CMs. Following injection of tamoxifen at P0, hearts harvested on postnatal days P7, P12 and P39 were immunostained for Ki67 (panel a), BrdU (panel b), and phosphohistone H3 (pH3) (panel c), plus GATA4 to identify CMs. Percentages of cell cycle-activated CMs were enumerated by blinded observers while scanning entire sections at 1000× magnification. Vertical lines indicate ±SEM. Statistical significance was determined using unpaired two-tailed t-tests.

FIG. 17 shows that Cre-recombinase alone does not alter percentages of BrdU-positive CMs in wild-type hearts. Kat5^{+/+} and Kat5^{+/+;Cre} neonatal mouse pups were injected with 250 μg tamoxifen at P0, followed by injection of BrdU at P11 or P40, and respectively harvesting hearts for histological analysis at P12 (panel a) or P41 (panel b). A minimum of 500 GATA4-positive nuclei (indicative of CM identity) were evaluated in each heart for co-expression of BrdU. Bars with scatter plots indicate the mean; vertical lines indicate ±SEM. Statistical significance was determined using an unpaired two-tailed t-test.

FIG. 18 shows the relative numbers of Ki67-positive CMs and non-CMs in Tip60-depleted hearts at P7 (panel a), P12 (panel b), or P39 (panel c). Cardiomyocytes were identified by staining GATA4. Counts are normalized to a total of 500 enumerated cardiomyocytes.

FIG. 19 shows the relative numbers of BrdU-positive CMs and non-CMs in Tip60-depleted hearts at P7 (panel a), P12 (panel b), or P39 (panel c). Cardiomyocytes were identified by staining GATA4. Counts are normalized to a total of 500 enumerated cardiomyocytes.

FIG. 20 shows the relative numbers of pH3-positive CMs and non-CMs in Tip60-depleted hearts at P7 (panel a), P12 (panel b), or P39 (panel c). Cardiomyocytes were identified by staining GATA4. Counts are normalized to a total of 500 enumerated cardiomyocytes.

FIG. 21 shows WGA-stained CMs at P12. Mice were injected with 250 μg tamoxifen at P0 followed by processing of hearts at P12. Sections were stained with FITC-conjugated wheat germ agglutinin (WGA), followed by photomicrography of six 400× fields containing CMs in transverse orientation. Panel a shows the average CM cross-sectional area determined in blind by processing photomicrographs with ImageJ software, wherein manually-thresholded CM cross-sectional areas were analyzed by particle analysis using constant settings of 600-infinity for pixels and 0.25-1.0 for circularity. A minimum of 250 CMs was evaluated in each heart. (Note: The lowball outlier in the control group was dysmorphic.) Panel b shows CM numbers that were estimated from total number of WGA-enclosed areas within these six fields, determined in blind using ImageJ software wherein CM cross-sectional areas were manually thresholded followed by particle analysis using constant settings of 600-infinity (pixels) and 0.25-1.0 (circularity). Vertical lines=±SEM. Statistical significance was determined using an unpaired two-tailed t-test with Welch's correction.

FIG. 22 shows anatomical data of Tip60-depleted mice subjected to MI. Kat5^{f/f} and Kat5^{Δ/Δ} mice were treated with tamoxifen on P0, subjected to MI at P7, and harvested on P39, followed by acquisition of anatomical data. Panels A, B, and C respectively show left ventricular mass, left ventricular wall thicknesses by echocardiography, and total heart weight. Panel D shows heart weight data normalized to body weight (BW), tibial length, dry lung weight (DL), and wet lung weight (WL). *P<0.05 vs. Kat5^{f/f}.

FIG. 23 shows that tamoxifen induces depletion of Tip60 in hearts with foxed Kat5 alleles. Adult Kat5^{f/f} and Kat5^{Δ/Δ} mice were injected with 40 mg/kg tamoxifen on three consecutive days, after which hearts were collected and processed for assessment of Tip60 mRNA and protein levels as described in Methods. Panel A shows depletion of Kat5 mRNA in Kat5^{Δ/Δ} hearts assessed by qRT-PCR 3-8 days after the first of three daily tamoxifen injections. Panel B (upper) is a representative western blot showing depleted levels of Tip60 protein in Kat5^{Δ/Δ} hearts collected 8-9 days after the first tamoxifen injection; the bar graph (lower) shows the extent of Tip60 depletion as assessed by quantitative densitometry. *P<0.05 vs. Kat5^{f/f}.

FIG. 24 shows that Tip60 depletion preserves function of infarcted mouse hearts. Panel A depicts the experimental timeline for the MI studies. Mice were injected with tamoxifen (40 mg/kg i.p.) on three consecutive days to deplete Tip60 in CMs beginning three days after induction of MI by left main coronary artery ligation (day 0). To assess heart structure and function, transthoracic echocardiography was performed at baseline and at the indicated intervals up to 28 days post-MI when hearts were harvested and processed for histological assessment. One day prior to harvest, mice were injected with BrdU (1 mg i.p.). Panels B-D shows indices of left ventricular function determined by echocardiography on the indicated days post-MI; fractional shortening (FS), left ventricle inner dimensions (LVIDs), and myocardial performance index (MPI) were preserved in Tip60-depleted (Kat5^{Δ/Δ}) hearts (N=8), but not in Kat5^{f/f} controls (N=5). Additional echocardiographic parameters are tabulated in Table 7. FIG. 33 shows that functional improvement was observed in both male and female mice. *P<0.05 vs. Kat5^{f/f}; ^{†}P<0.05 vs baseline value (0 days post-MI).

FIG. 25 shows that depletion of Tip60 after MI reduces scarring. Panel A: Representative trichrome-stained cross-sections obtained at intervals of 0.8 mm along the basal-apical axis of control (Kat5^{f/f} and Tip60-depleted (Kat5^{Δ/Δ}) hearts at 28 days post-MI; blue stain denotes area of the scar. Panel B: Infarct scar size quantified by measurement of area and midline length.

FIG. 26 shows activation of the cell cycle in CMs of infarcted Tip60-depleted hearts. Quantification of heart sections immunostained for Ki67, BrdU, and pH3 revealed that cell cycle activity in CMs within both the infarct border and remote zones was increased in Tip60-depleted (Kat5^{Δ/Δ}) hearts at 28 days post-MI, compared to controls (Kat5^{f/f}). Panel A shows representative 400× microscopic fields co-immunostained to detect cTnT (to identify CMs) and Ki67. White and yellow arrows in Panel A denote examples of Ki67-positive CMs and non-CMs, respectively. Panels B-D respectively show quantification of Ki67-positive (Panel B), BrdU-positive (Panel C), and pH3-positive (Panel D) CMs per 200× field in the border and remote zones relative to the zone of infarct; border and remote zones are anatomically defined as described in Methods. Examples of BrdU/cTnT- and pH3/cTnT-positive CMs are shown in FIG. 27.

FIG. 27 shows SMA expression in CMs in the border zone of infarcted Tip60-depleted hearts. Panels A-C are representative images of the infarct border zone at 28 days post-MI in hearts of control (Kat5^{f/f}, left) and Tip60-depleted (Kat5^{Δ/Δ}, right) mice, immunostained for SMA and viewed at 400× (Panel A), 100× (Panel B), and 40× (Panel C) magnification. Striated, SMA-positive CMs are aligned along the border zone of Tip60-depleted hearts; striations, which are most apparent under 400× magnification (Panel A), indicate that SMA is expressed in CMs.

FIG. 28 shows reduced numbers of TUNEL-positive and cleaved caspase-3-positive cells in the remote zone of infarcted Tip60-depleted hearts. Control (Kat5^{f/f}) and Tip60-depleted (Kat5^{Δ/Δ}) hearts were histologically processed to detect TUNEL-positive and cleaved caspase-3-positive cells at 28 days post-MI. Panel A: Representative 600× TUNEL images from the remote zone; all TUNEL signals were nuclear (verified by DAPI stain). Panel B: Enumeration of TUNEL-positive cells (exclusive of cellular identity) in sections from the border and remote zones relative to area of infarction. Panel C: Representative cleaved caspase-3-stained 600× images taken from the border zone and remote zone of infarcted Kat5^{Δ/Δ} hearts. Panel D: Enumeration of cleaved caspase-3-positive cells (exclusive of cellular identity) in the border and remote zones.

FIG. 29 shows dysfunction and increased numbers of TUNEL-positive cells in the remote zone of infarcted Kat5^{+/+;Myh6-merCremer} mice. Wild-type (Kat5^{+/+}) and Kat5^{+/+;Myh6-merCremer} mice were subjected to MI and tamoxifen treatment as described in FIG. 24A. Panel A shows indices of left ventricular function (FS, LVIDs, and MPI; N=5/group) determined by echocardiography on the indicated days post-MI; dysfunction was observed in both experimental groups (FIG. 37 provides the results separated by sex). Additional echocardiographic parameters are tabulated in Table 8. Panel B shows representative 600-x TUNEL images and enumeration of TUNEL-positive cells (exclusive of cellular identity) in sections from the border and remote zones relative to area of infarction. *P<0.05 vs. Kat5^{f/f}; ^{†}P<0.05 vs baseline value (0 days post-MI).

FIG. 30 shows a schematic of Tip60-regulated pathways that may inhibit proliferation and activate apoptosis in cardiomyocytes. Scheme depicting molecular pathways known to inhibit CM proliferation (p53,²⁶ Atm^{29, 30} p21,¹⁴ Tert⁶⁰) that are regulated by Tip60 in other cell types. Ac, acetylation; P, phosphorylation.

FIG. 31 shows the cardiac function and longevity of non-injured Tip60-depleted mice. Cardiac structure and function of naïve non-infarcted Tip60-depleted (Kat5^{Δ/Δ}) mice determined by echocardiography were unaffected 4 weeks (28 days) after the first of three tamoxifen (40 mg/kg i.p.) injections to deplete Tip60. By 20 weeks, mild dysfunction became apparent when the mice began to die. Panels A and B show FS and MPI data, respectively. Additional echocardiographic data are tabulated in Table 6. Panel C presents Kaplan-Meier curves. *P<0.05 vs. Kat5^{f/f}; ^{†}P<0.05 vs baseline.

FIG. 32 shows a trend toward improved post-MI survival in Tip60-depleted mice using Kaplan-Maier curves comparing survival of infarcted control (Kat5^{f/f}) and Tip60-depleted (Kat5^{Δ/Δ}) mice.

FIG. 33 shows improved post-MI functional recovery in male and female Tip60-depleted mice. Echocardiographic data indicate that indices of left ventricular function (FS, LVIDs, & MPI) were preserved in both male (upper) and female (lower) Tip60-depleted)(Kat5^{Δ/Δ}) mice. *P<0.05 vs. Kat5^{+/+}; ^{†}P<0.05 vs baseline value (0 days post-MI).

FIG. 34 shows attenuated CM hypertrophy in the border zone of infarcted Tip60-depleted hearts. Sections from Tip60-depleted (Kat5^{Δ/Δ}) and control (Kat5^{f/f}) hearts at 28 days post-MI were stained with wheat germ agglutinin (WGA) to outline CM boundaries, followed by ImageJ processing to estimate CM size by calculating numbers of pixels within CMs that were in transverse orientation. Panel A: Representative cross-sectional images of WGA staining taken at 400×. Panel B: Quantitation of CM size measured by the average number of pixels in transversely sectioned CMs.

FIG. 35 shows examples of immunostains for BrdU (Panel A) and pH3 (Panel B), each co-stained for cTnT to identify CMs, at 28 Days post-MI in hearts from control (Kat5^{f/f}, left) and Tip60-depleted (Kat5^{Δ/Δ}, right) mice. Photomicrographs were made at 400× magnification. White arrows denote examples of CMs positive for BrdU or pH3; yellow arrows denote non-CMs.

FIG. 36 shows activation of the cell cycle in non-CMs of Tip60-depleted hearts. Quantification of heart sections immunostained for Ki67, BrdU, and pH3 indicated that cell cycle activity in non-CMs was increased in Tip60-depleted (Kat5^{Δ/Δ}) hearts at 28 days post-MI. Panels A-C respectively show quantification of Ki67-positive (Panel A), BrdU-positive (Panel B), and pH3-positive (Panel C) non-CMs per 200× field in the border and remote zones relative to the infarct zone.

FIG. 37 shows echocardiography results from the infarction studies with Kat5^{+/+} and Kat5^{+/+;Myh6-merCremer} mice separated by sex, which demonstrate that Cre-recombinase-mediated cardiac dysfunction does not exhibit gender bias. *P<0.05 vs. Kat5^{+/+}; ^{†}P<0.05 vs baseline value (0 days post-MI).

FIG. 38 shows a comparison of scarring in hearts of wild-type (Kat5^{+/+}) and Kat5^{+/+;Myh6-merCreMer} mice 28 days post-MI, which demonstrates that Cre-recombinase alone does not affect scarring at 28 days post-MI. Panel A: Representative trichrome-stained images of cross-sections obtained at ˜0.8 mm intervals along the basal-apical axis of 28 day post-MI hearts. Blue stain denotes area of the scar. Panel B: Scar size quantified by measurements of area (left) and midline length (right).

FIG. 39 shows an assessment of cell cycle activity in CMs and non-CMs of infarcted wild-type (Kat5^{+/+}) and Kat5^{+/+;Myh6-merCreMer} hearts, which demonstrates that Cre-recombinase alone does not affect cell cycle activation at 28 days post-MI. Quantitative immunostaining of Ki67 showed that cell cycle activity in CMs (cTnT+) or non-CMs (cTnT−) of wild-type mice bearing the Myh6-merCremer transgene at 28 days post-MI was not different from wild-type controls. Representative micrographs were obtained at 200× magnification. White and yellow arrows respectively denote examples of Ki67-positive CMs and non-CMs.

FIG. 40 shows drugs and compounds that act as Tip60 (Tat interactive protein, 60 kDa) inhibitors. [Adapted from: Biochem. Soc. Trans. 44:979-986 (2016)]

### DETAILED DESCRIPTION

The present disclosure provides compositions and methods for inducing cardiomyocyte (CM) proliferation, both in vitro and in vivo. The methods may be used in the treatment of patients suffering from cardiac disorders, particularly those who have suffered damage or loss of cardiac muscle tissue, for example, subjects that have suffered from a myocardial infarction (MI).

Contractile dysfunction and mortality associated with ischemic heart disease are caused by a massive loss of cardiomyocytes (CMs), a cell-type that is essentially non-regenerable due to its pronounced state of proliferative senescence. As detailed in a recent review article,¹ the most promising therapeutic interventions for re-muscularizing the myocardium are (i) transplantation of CMs derived from pluripotent stem cells, (ii) inducing new CMs via re-programming of non-CMs, and, (iii) expanding numbers of pre-existing CMs by inducing their re-entry into the cell cycle.¹ The latter approach, which has received increasing attention during the past decade, has been attempted using strategies including the augmentation of pro-proliferative factors and the depletion of cell cycle inhibitory factors. Regarding the latter, the need to relieve the CM cell cycle from multiple layers of inhibitors that induce and maintain profound proliferative senescence was recently cited.² Experimental depletion of inhibitors including tumor suppressor proteins such as retinoblastoma,³ meis1,⁴ glycogen synthase kinase-3 (Gsk-3),⁵ and components of hippo signaling⁶ has been shown to permit the resumption of CM proliferation to varying degrees. Because the cell cycle contains multiple points of inhibition, it is reasonable that the co-depletion of multiple factors, or of single factors possessive of pleiotropic inhibitory function, would enhance the regenerative response.

To induce the proliferation of pre-existing CMs, the present invention targets Tip60 (Tat-interactive protein U60U kD). Tip60 is a protein with pleiotrophic functions that contains a chromodomain as well as an acetylase domain. Tip60 is widely conserved in mammals and is encoded by the lysine acetyltransferase-5 (Kat5) gene, which is expressed in all tissues examined including the heart.²² Tip60 is a pan-acetylase that regulates cellular functions including apoptosis, the DNA damage response (DDR), and cell cycle progression. At the molecular level, Tip60 acetylates Atm⁷⁻⁹ (ataxia-telangiectasia mutated), a kinase that consequently undergoes auto-phosphorylation to induce the DNA damage response (DDR) in various cell types. In CMs, activated Atm (phosphorylated Atm or pATM) ultimately causes proliferative senescence and inability to regenerate. Tip60 also acetylates p53,^{10,11} activating apoptosis. More recently it was shown that Tip60 regulates intracellular levels of p21,¹⁴ Tert polymerase,¹⁵ and aurora-B kinase,¹⁶ contributing to the maintainance proliferative senescence. These functions of Tip60, combined with its depletion in ten human cancers including lymphoma,¹⁷ breast,^{18, 19} and prostate carcinoma,^{20, 21} have accorded its inclusion as a member in the tumor suppressor gene database.

The inventors previously investigated Tip60's role in the heart by globally and conditionally ablating the gene encoding Tip60, Kat5, respectively in Kat5^{+/-} or Kat5^{flox/flox} mice ((Fisher et al PLoS One. 2012; 7:e31569. doi: 10.1371/journal.pone.0031569, PMID: 22348108; Fisher e al PLoS One. 2016; 11(10):e0164855. doi: 10.1371/journal.pone.0164855, PMID: 27768769), revealing that modest depletion activates the cardiomyocyte (CM) cell cycle, whereas chronic depletion kills CMs by week 12, a phenomenon preceded by increased CM density at postnatal day 15 (P15).

In the present application, the inventors demonstrate the effects of conditionally depleting Tip60 for a finite duration, i.e. using conditions wherein only the immediate-early effects of Tip60 depletion are observed. To do so, they employed a mouse model in which Kat5 is experimentally and conditionally disrupted, enabling depletion of Tip60 protein in CMs on demand. The inventors show that, in both neonatal (Example 1) and adult hearts (Example 2), depletion of Tip60 (a) depresses the DDR and (b) permits increased CM proliferation. In the adult heart, following the experimental induction of myocardial infarction, depletion of Tip60 remarkably improves cardiac function, to a level that significantly exceeds function exhibited by controls (Example 2). These results suggest that inhibition of Tip60 is cardioprotective, and that inhibiting Tip60 using a small molecule drug may temporarily permit CM proliferation to regenerate the infarcted myocardium (Example 3).

Accordingly, the present disclosure provides methods that utilize a Tip60 inhibitor to induce CM proliferation. In certain embodiments, the Tip60 inhibitor is used to induce the regeneration of heart tissue in a patient in need thereof. In other embodiments, the Tip60 inhibitor is used to prevent, treat or alleviate a disease or injury of cardiac cells.

### Methods:

The present disclosure provides a method for inducing proliferation of adult CMs. The method comprises transiently contacting the adult CMs with an amount of a Tip60 inhibitor that is sufficient to induce proliferation of CMs. The CMs can be in vitro or in vivo. In a preferred embodiment, the CMs are in vivo in a patient in need of CM regeneration. In some embodiments, the method comprises administering to the patient a therapeutically effective amount of Tip60 inhibitor transiently to induce in vivo proliferation of CMs.

As discussed above, persistent activity of a Tip60 inhibitor in cells may be lethal, causing cell death. Therefore, in the methods described herein, Tip60 inhibition should be transient, e.g., should cause Tip60 inactivity for a limited duration in CMs. Therefore, suitable Tip60 inhibitors would be inhibitors that can be cleared from the subject after having provided their therapeutic effect for a sufficient amount of time. For example, the Tip60 inhibitor should be present for a sufficient time in CMs to inhibit Tip60 at a level sufficient to permit CMs to undergo cell division and proliferate, resulting in an increased number of CMs within the subject. As most drugs and small molecules have a definitive duration of action defined by their in vivo half-life, one skilled in the art would be able to determine and adjust the dosing and administration to provide an effective concentration of a drug to inhibit Tip60 transiently in CMs for a sufficient time to permit CM proliferation. The drug may need to be given to inhibit Tip60 for up to several days until the maximum number of new CMs are formed to restore heart contractile function back to normal. For example, but not limited to, the drug may be given to a subject for e at least about three days, at least about one week, at least about two weeks, at least about three weeks, at least about four weeks, at least about five weeks, at least about six weeks, at least about seven weeks, at least about 8 weeks, at least about nine weeks, at least about ten weeks, at least about 11 weeks, at least about 12 weeks, or any time-frame in-between that is sufficient to allow for new CMs formation and/or sufficient time to restore heart contractile function.

The term “cardiac cell” as used herein includes not only cardiomyocytes, but also other cell types that comprise functional cardiac tissue. Exemplary cardiac cells include, but are not limited to, endocardial cells, pericardial cells, cardiomyocytes, epicardial cells, and mesocardial cells.

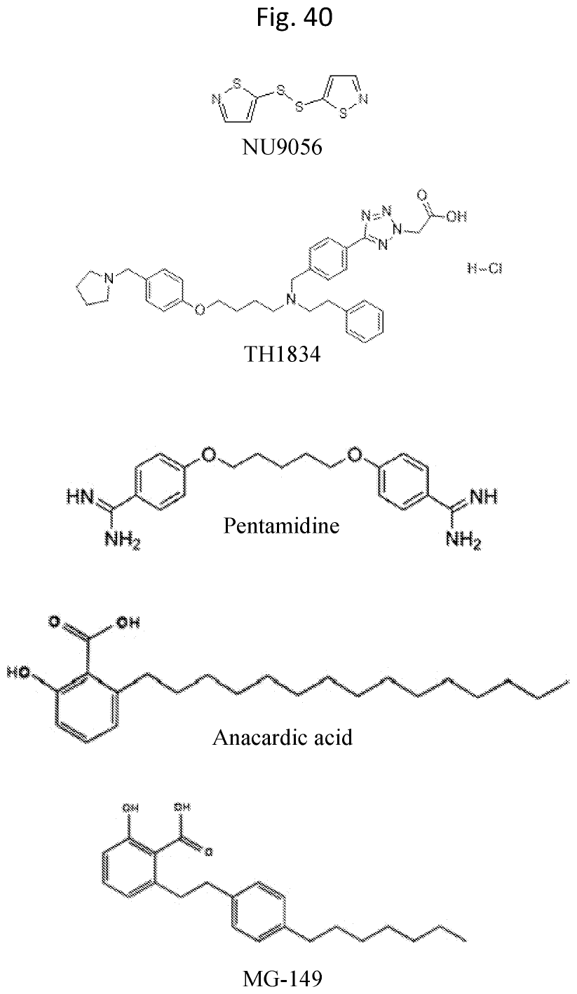

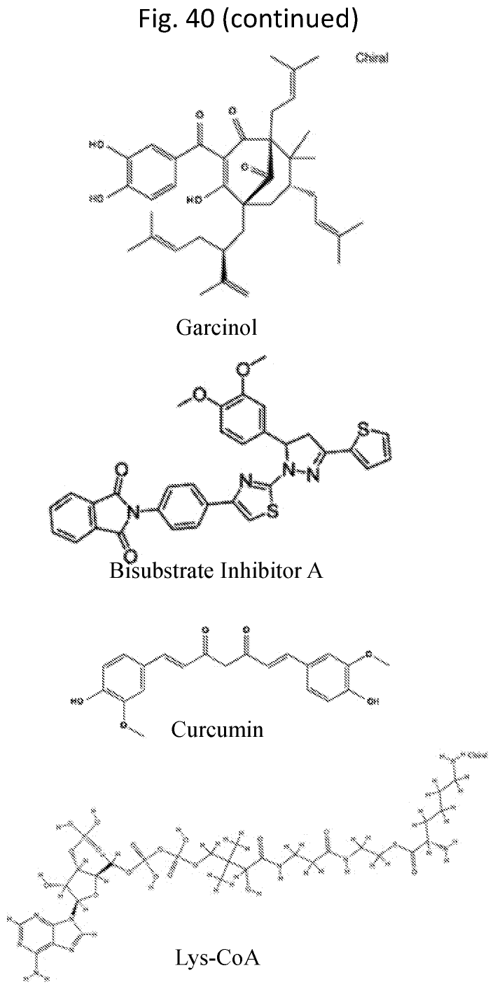

Suitable Tip60 inhibitors are known by one skilled in the art and commercially available. Suitable Tip60 inhibitors include, but are not limited to, for example, NU9056 (Tocris biotechne, Minneapolis Minn.), TH1834 (Axon Medchem, Reston, Va.), Pentamidine, Anacardic acid, MG-149, Garcinol, Bisubstrate Inhibitor A, Curcumin, LysCoA (See FIG. 40), among others. In preferred embodiments, the Tip60 inhibitor is NU9056.

The methods of the present invention can be used to treat a wide range of injuries and diseases characterized by a decrease in cardiac function. Suitable diseases or injuries include, but are not limited to, for example, myocardial infarction, myocardial ischemia, myocarditis, myocardial damage from myocardial infarction; atherosclerosis; coronary artery disease; obstructive vascular disease; dilated cardiomyopathy; heart failure; myocardial necrosis; valvular heart disease; non-compaction of the ventricular myocardium; hypertrophic cardiomyopathy; exposure to a toxin, exposure to cancer chemotherapeutics or radiation treatment, exposure to an infectious agent, or mineral deficiency. In one preferred embodiment, the disease or injury is myocardial infarction.

As used herein, the terms “contacting” or “administering” refers to any suitable method of bringing the Tip60 inhibitor into contact with the cardiac cells. For example, in some embodiments, the cardiac cells are within a patient and the contacting step comprises administering to the patient the Tip60 inhibitor. For such in vivo applications, any suitable method of administration may be used. For example, local administration or systemic (e.g., oral or intraveneous) administration is contemplated.

The inhibitors and compositions, including the pharmaceutical compositions described in the present application can be administered systemically or locally. Locally administered compositions can be delivered, for example, to the pericardial sac, to the pericardium, to the endocardium, to the great vessels surrounding the heart (e.g., intravascularly to the heart), via the coronary arteries, or directly to the myocardium. Delivery may be accomplished, for example, using a syringe, catheter, stent, wire, or other intraluminal device. For example, intracardial injection catheters can be used to deliver the inhibitors or compositions of the invention directly to a specific tissue. When the inhibitor is to be delivered to repair damaged tissue (e.g., damaged myocardium), it may be delivered directly to the site of damage or delivered to another site at some distance from the site of damage.

In one embodiment, when treating an acute disease or cardiac injury, the inhibitors or compositions described herein can be administered to the patient within at least 5 days of the diagnosis of the acute disease or injury, preferably within 1 day of the acute onset of disease. In other embodiments, the inhibitors or compositions described herein can be administered at any time after the onset of cardiac disease or injury, for example, they can be used to treat a patient with long-standing, chronic disease such as heart failure, at any time after diagnosis.

The disclosure further provides a method of regenerating heart tissue within a patient in need thereof. The method comprises administering an effective amount of a Tip60 inhibitor to transiently induce proliferation and regeneration of heart tissue. A suitable patient in need includes patients who have cardiac disease or who have cardiac injury, including myocardial infarction and ischemia of cardiac tissue. Regenerating heat tissue can include an increase in cardiac muscle cells and restoring or improving heart function (e.g., as measured bia echocardiography or reduced scarring).

The disclosure further provides a method of treating a subject with cardiac injury, the method comprising administering a therapeutically effective amount of a Tip60 inhibitor to treat the cardiac injury. In some examples, the Tip60 inhibitor is administered transiently.

The methods described herein for regenerating the proliferation of pre-existing cardiomyocytes comprise transiently administering at least one Tip60 inhibitor in an amount effective to regenerate or proliferate the existing cardiomyocytes. In further embodiments, the methods can be combined with administering a glycogen synthase kinase (GSK) inhibitor to the patient. Not to be bound by theory, but it is believed by the inventors that Gsk directly activates Tip60 in cardiomyocytes. Hence, a GSK inhibitor used in combination with a Tip60 inhibitor should result increase the efficacy of the Tip60 inhibitor, promoting increased cardiomyocyte proliferation and regeneration.

Glycogen synthase kinase (GSK)-3 is a serine/threonine kinase that phosphorylates either threonine or serine amino acids within proteins. This phosphorylation permits a variety of biological activities such as cell proliferation, glycogen metabolism, cell signaling, cellular transport, and others. GSK is known to phosphorylate, and thereby activate, Tip60. GSK inhibitors are known in the art and include, but are not limited to, lithium ion, valproic acid, iodotubercidin, Naproxen, Cromolyn, Famotidine, curcumin, olanzaprine, pyrimidine derivatives. Suitable GSK-3 inhibitors are commercially available at Tocris Bioscience (Minneapolis, Minn.) or Selleckchem (for example, 3F8, A1070722, Alsterpaullone, AR-A014418, BIO, BIO-acetoxime, CHIR 98014, CHIR 99021, CHIR 99021 trihydrochloride, Indirubin-3′-oxime, Kenpaullone, lithium carbonate, NSC 693869, SB216763, SB415286, TC-G 24, TCS 21311, TDZD-8, TWS119, SB216763, 5-Bromoindole, 2-D08, AZD1080, LY2090314, IM-12, Indirubin, Bikini, 1-Azakenpaullone, and Tideglusib among others).

For purposes of the present invention, “treating” or “treatment” describes the management and care of a subject for the purpose of combating the disease, condition, or disorder. Treating includes the administration of an inhibitor or composition of present invention to prevent the onset of the symptoms or complications, alleviating the symptoms or complications, or eliminating the disease, condition, or disorder. Suitably, the disease is a cardiac disease or injury that results in a decrease in cardiac function and/or a decrease in cardiomyocytes within the patient. Suitably, the term treatment encompasses (a) relieving one or more symptoms of cardiac disease or injury, (b) increasing the number of cardiomyocytes within a patient, (c) increasing cardiac function within a patient, (d) increasing cardiac muscle mass within a patient, (e) decreasing ischemia within the heart of a patient having had myocardial infarction, among others. In one embodiment, treatment encompasses increasing the number and function of cardiomyocytes in a patient. Function of cardiomyocytes may be assessed, for example using echocardiography.

A “therapeutically effective amount” is an amount sufficient to effect beneficial or desired results, including clinical results (e.g., amelioration of symptoms, achievement of clinical endpoints, and the like). An effective amount can be administered in one or more administrations. In terms of a disease state, an effective amount is an amount sufficient to ameliorate, stabilize, or delay development of a disease. For any compound described herein, the therapeutically effective amount can be initially estimated from cell culture assays. For example, a dose can be formulated in animal models to achieve a circulating concentration of the Tip60 inhibitor as to be therapeutically effective. Such information can then be used to determine useful doses in humans (see, e.g., Washburn et al, 1976, “Prediction of the Effective Radioprotective Dose of WR-2721 in Humans Through an Interspecies Tissue Distribution Study” Radial. Res. 66:100-5).

In some embodiments, the present invention also provides methods of in vitro proliferation of adult cardiomyocytes in culture, the method comprising contacting the cardiomyocytes transiently with an inhibitor of Tip60 in an effective amount to proliferate cardiomyocytes in culture. The present invention provides for a method of inducing myocardial cell proliferation in vitro, as well as for myocardial cell cultures produced by this method. In a preferred embodiment, the invention provides for human myocardial cell cultures. The myocardial cell cultures of the invention may be used to study the physiology of cardiac muscle and to screen for pharmaceutical agents that may be useful in the treatment of heart disease. Furthermore, the cultures of the invention may be used to provide myocardial cells that may be transplanted or implanted into a patient that suffers from a cardiac disorder.

### Compositions:

Compositions or medicaments for inducing the proliferation of cardiac cells (e.g., cardiomyocytes), treating cardiac injury (e.g., myocardial infarction), or regenerating heart tissue are also contemplated. Suitable compositions comprise a Tip60 inhibitor. The compositions are administered in an amount effective to provide a transient inhibition of Tip60 within the cardiomyocytes within the subject.

In one embodiment, the present invention provides a medicament for use in a method of proliferating cardiomyocytes in a patient, the method comprising administering an effective amount of Tip60 inhibitor to proliferate cardiomyocytes.

In some embodiments, the compositions comprise a pharmaceutically acceptable carrier. “Pharmaceutically acceptable” carriers are known in the art and include, but are not limited to, for example, suitable diluents, preservatives, solubilizers, emulsifiers, liposomes, nanoparticles and adjuvants. Pharmaceutically acceptable carriers are well known to those skilled in the art and include, but are not limited to, 0.01 to 0.1M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. Water is not contemplated as a suitable physiologically acceptable carrier. In some embodiments, additional components may be added to preserve the structure and function of the inhibitors of the present invention, but are physiologically acceptable for administration to a subject.

The compositions used with the present invention can be sterilized by conventional, well-known sterilization techniques. The compositions may contain pharmaceutically acceptable additional substances as required to approximate physiological conditions such as a pH adjusting and buffering agent, toxicity adjusting agents, such as, sodium acetate, sodium chloride, potassium chloride, calcium chloride, sodium lactate, and the like. Compositions of the present disclosure may include liquids or lyophilized or otherwise dried formulations and may include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the polypeptide, complexation with metal ions, or incorporation of the material into or onto particulate preparations of polymeric compounds such as polylactic acid, polyglycolic acid, hydrogels, etc., or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). In some embodiments, the inhibitors are provided in lyophilized form and rehydrated with sterile water or saline solution before administration.

Kits for carrying out the methods described herein are also provided. The kits provided may contain the necessary components in which to carry out one or more of the above-noted methods. In one embodiment, the kit comprises a composition comprising one or more Tip60 inhibitors and instructions for use.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit's interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim.

In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention. The present invention has been described in terms of one or more preferred embodiments, and it should be appreciated that many equivalents, alternatives, variations, and modifications, aside from those expressly stated, are possible and within the scope of the invention.

Each publication, patent, and patent publication cited in this disclosure is incorporated in reference herein in its entirety. The present invention is not intended to be limited to the following examples, but encompasses all such modifications and variations as come within the scope of the appended claims.

### EXAMPLES

### Example 1: Cell Cycle Activation and Proliferation in Tip60-Depleted Neonatal Cardiomyocytes

In two previous studies (Fisher et al PLoS One. 2012; 7:e31569. doi: 10.1371/journal.pone.0031569, PMID: 22348108; Fisher et al PLoS One. 2016; 11(10):e0164855. doi: 10.1371/journal.pone.0164855, PMID: 27768759), we found that modest depletion of Tip60 activates the cardiomyocyte (CM) cell cycle, whereas chronic depletion of Tip60 kills CMs by week 12, a phenomenon preceded by increased CM density at postnatal day 15 (P15). These findings, considered vis-à-vis findings of others that Atm activation of the DNA damage response (DDR) pathway in the early neonatal period causes CM proliferative senescence, caused us to hypothesize that Tip60 induces DDR in CMs by phosphorylating Atm, and accordingly that Tip60 depletion should increase the extent and/or duration of postnatal CM proliferation.

In this Example, we report western blot evidence for increased levels of phosphorylated Atm (pAtm), and especially bulk Atm, in hearts of wild-type mice beginning at postnatal day P2. In Kat5^{flox/flox;Myh6-mCrem} mice treated with tamoxifen to deplete Tip60 in CMs, immunofluorescent microscopy revealed that Tip60 depletion occurred concomitant with reduced numbers of pAtm-positive CM nuclei during postnatal development (P7-P39). Effects of Tip60 depletion on cell cycle regulatory genes revealed significantly decreased levels of mRNAs encoding cell cycle inhibitors (Meis1, p27), concomitant with a trend toward increased levels of mRNAs encoding G₂-phase activators (cyclins A2, B1; Cdk1). Immunostaining for Ki67, BrdU and phosphohistone H3 revealed that these changes were accompanied by cell cycle activation in non-CMs as well as CMs, the latter being accompanied by significantly increased levels of mononuclear diploid cardiomyocytes at P12. However, although cell cycle activation in Tip60-depleted CMs was increased over control levels at each developmental stage, both genotypes progressively decreased with increasing age, indicating only partial relief from replicative senescence.

### Materials and Methods:

Animal care & use: This investigation adhered to the National Institutes of Health (NIH) Guide for the Care and Use of Laboratory Animals (NIH Pub. Nos. 85-23, Revised 1996). All protocols described in the authors' Animal Use Application (AUA #000225), which were approved by the Medical College of Wisconsin's Institutional Animal Care and Use Committee (IACUC), were adhered to in this study. The IACUC has an Animal Welfare Assurance status from the Office of Laboratory Welfare (A3102-01).

Preparation of mice containing floxed Kat5 alleles, wherein Cre-recombinase removes two-thirds of the Tip60 coding sequence including the chromo and acetyltransferase domains, was recently reported in detail [5]. For the experiments described here, these mice were mated with a line obtained from the Jackson Laboratory (Jax #005650) that expresses an α-myosin heavy chain (Myh6)-driven merCremer-recombinase transgene that, upon administration of tamoxifen, enters the nucleus to recombine the floxed alleles [13].

In these experiments, neonatal mice were administered a single injection containing 250 μg tamoxifen (Sigma #T5648) suspended in 6.25% ethanol/sunflower oil into the scruff of the neck on the day of birth (P0). On the day prior to harvest, mice were injected 0.7 mg 5′-bromodeoxyuridine (BrdU). On the days of harvest hearts were perfused with ˜3 ml cardioplegic solution (25 mM KCl/5% dextrose in PBS) followed by apportionment of transversely-sectioned portions for histology, RNA isolation and western blotting, respectively into ice-cold 4% paraformaldehyde (PFA), TRIzol Reagent (Thermo-Fisher #15591626), and RIPA buffer (Thermo-Fisher #89901) containing Halt anti-protease/anti-phosphatase cocktail (Thermo-Fisher #78440). Tissue for histology was fixed in ice-cold 4% PFA overnight, transferred to 70% EtOH for a minimum of one overnight, followed by embedding in paraffin. Samples for RNA were homogenized with a teflon pestle, and, samples for protein were minced and sonicated; both were stored at −80° C. until final processing.

Myocardial infarction and echocardiography of neonatal mice: Pups were removed from the nursing mother and placed in a separate cage away from the mothers during the surgical procedure. P7 mice were anesthetized on ice by hypothermia for ˜3-5 minutes. To produce hypothermic anesthesia, the mice were placed on a surgical gauze sitting on a bed of ice. The level of anesthesia was assessed by the toe pinch reflex response. Once anesthetized, the pups were positioned on their right side and the left forepaw raised away from the chest. The chest was cleaned with chlorhexidine followed by 70% ethanol a total of three times. The mice were placed onto the stage of a Fisher Scientific™ Stereomaster™ Microscope that utilizes Fiber-Optic ring lights that produce little heat.

A lateral thoracotomy was performed ˜3 mm at the fourth intercostal space exposing the heart. An 8-0 prolene suture was threaded through the mid-ventricle under the artery via microscope guidance. Ischemia was induced by tying the suture, being careful not to damage the vessel or myocardium. Successful occlusion was verified by blanching of the myocardium below the ligature. Once the ligature was in place, the incision was closed with two stitches using 8-0 nonabsorbable prolene to close the ribs, muscle, and skin. Subsequently, the pups were removed from the ice dish, placed near a heat lamp until fully recovered, and then returned to the mother.

On the day before harvest (P39), mice were injected (1 mg) intraperitoneally with BrdU and subjected to echocardiography assessment. On the day of harvest, mice were euthanized with CO₂ and hearts were perfused with cardioplegic solution (25 mM KCl/5% dextrose in PBS) followed by 4% paraformaldehyde (PFA). After overnight fixation in PFA, hearts were transferred to 70% EtOH, followed by embedding in paraffin.

Genotyping was performed by PCR in 20 μl reactions that included 2× GoTaq Green Mastermix (Promega #M7123), 1.1 mM MgCl₂, 0.5 μM each primer, 0.5 μM internal control primers, and 4.0 μl template. Templates consisted of 1,200 g supernatants of ear tissue (punches) or tail tip samples that had been boiled for 10 minutes in 0.3 ml 10 mM NaOH/1 mM EDTA. Sequences of primer pairs used for PCR are listed in Table 1. PCR products were amplified in an AB Applied Biosystems GeneAmp PCR System 9700 using the following programs: for LoxP, one 5 minute cycle at 95° C., thirty-five cycles at 94° C. 30 sec/61° C. 45 sec/72° C. 45 sec, followed by one 10 minute cycle at 72° C.; for Myh6-merCremer, one 5 minute cycle at 95° C., thirty-five cycles at 94° C. 30 sec/54° C. 45 sec/72° C. 45 sec, followed by one 10 minute cycle at 72° C. Amplicons were separated at 80-95 V for two hours in 2% agarose and imaged after ethidium bromide staining.

TABLE 1
Primers & Probes
for PCR Genotyping

| | | Lough lab | Amplicon | Annealing |
|---|---|---|---|---|
| Allele | Sequence (5′-3′) and Working Conc. | Identifier | (bp) | ° C. |
| LoxP | FWD 0.5 μM | LFNF-fwd | 359 LoxP | 67 |
| in intron 2 | AGGGAGTCAACGATCGCACGGGAGG | | 258 WT | |
| | (SEQ ID NO: 1) | | | |
| | REV 0.5 μM | LFNF-rev | | |
| | CACAGACAGGGAGTCTTAGCCAGGG (SEQ | | | |
| | ID NO: 2) | | | |

Cycling Details: 94° C. 1 min, then 35 cycles of 94° C. 45 sec/67° C. 35 sec/
72° C. 60 sec, then 72° C. 10 mi

| | | | | |
|---|---|---|---|---|
| LoxP | FWD 0.5 μM | d813 | 785 LoxP | 56 |
| in intron 11 | CTGTGTCTTCTGGCCAAGTGTT | | 684 WT | |
| | (SEQ ID NO: 3) | | | |
| | REV 0.5 μM | 96c | | |
| | TCGGTTCTCAGAGACTAGC | | | |
| | (SEQ ID NO: 4) | | | |

Cycling Details: 94° C. 3 min, then 35 cycles of 94° C. 45 sec/56° C. 35 sec/
72° C. 60 sec, then 72° C. 10 min

| | | | | |
|---|---|---|---|---|
| Myh6-Cre | FWD 1.0 μM | per Jackson | ~100 | 52 |
| transgene | GCGGTCTGGCAGTAAAAACTATC | Lab | | |
| | (SEQ ID NO: 5) | #009074 | | |
| | REV 1.0 μM | | | |
| | GTGAAACAGCATTGCTGTCACTT | | | |
| | (SEQ ID NO: 6) | | | |

Cycling Details: Cycling Details: 94° C. 3 min, then 35 cycles of 94° C.30 sec/
52° C. 60 sec/72° C. 60 sec, then 72° C. 2 min
for Taqman qRT-PCR

| Gene Target | Taqman Probe Kit (Thermo-Fisher catalog #) |
|---|---|
| Gapdh | Mm99999915_g1 |
| Rp137α | Mm01546394_s1 |
| Kat5 (Tip60) | Mm01231512_m1 |
| Ccna2 (Cyclin A2) | Mm00438063_m1 |
| Ccnb1 (Cyclin B1) | Mm03053893_gH |
| Ccnd1 (Cyclin D1) | Mm00432359_m1 |
| Ccnd2 (Cyclin D2) | Mm00438070_m1 |
| Cdk1 | Mm00772472_m1 |
| Cdk4 | Mm00726334_s1 |
| Cdkn1a (p21) | Mm00432448_m1 (4930567H1+) |
| Cdkn1b (p27) | Mm00438168_m1 |
| Meis1 | Mm00487664_m1 |
| Myh7 | Mm00600555_m1 |
| Runx1 | Mm01213404_m1 |

Quantitative RT-PCR (qPCR): Heart tissue in Trizol was purified using PureLink RNA Mini-Kits (Invitrogen #12183018A), including a genomic DNA removal step (PureLink DNase; Thermo-Fisher #12185-010) according to the manufacturer's instructions. RNA Yield & Quality were determined using a Eppendorf Biophotometer Plus Instrument.

cDNA was synthesized as follows. After diluting an RNA sample from each heart so that precisely 1.0 μg was suspended in 14 μl nuclease-free distilled water (NFDW), 4.0 μl 5× VILO reaction mixture (Invitrogen #100002277) were added. To start the reverse-transcription reaction, 2.0 μl 10× SuperScript Enzyme Mix (Invitrogen #100002279) were added, followed by incubation for 10 minutes at 25° C., 60 minutes at 42° C., and 5 minutes at 85° C. cDNA templates were diluted with NFDW to a concentration of 6.25 ng/μl and stored at −20° C.

qPCR was carried-out by subjecting each biological replicate (i.e. sample from each individual heart) to triplicate determinations. Each reaction was performed in a total volume of 20 μl using 96-well arrays, each well containing 1× Taqman Fast-Advanced Master Mix (Thermo-Fisher #4444557), 1× Taqman Probe Kit (Table 1), and 25 ng cDNA as template. The arrayed samples were amplified in a Bio-Rad CFX96 Real Time System (C1000 Touch) programmed as follows: 2 minutes at 50° C.→0:20 minutes at 95° C.→0:03 min at 95° C.→0:30 at 60° C.; the last two steps were repeated 39 times. Results were processed using Bio-Rad CFX Manager 3.1 software.

Western blotting: Blots were prepared with total protein extracted from the superior third of each heart from which the atria had been removed. Upon harvesting in ice-cold RIPA buffer (Thermo-Fisher #89900) containing Halt Protease Inhibitor (Thermo-Fisher #1861281), samples were minced and sonicated (Misonix Sonicator 3000) for 10 seconds at an output setting of 2.5. Total protein concentration was determined using the standard Bradford Assay (Bio-Rad #500-0006) and samples were diluted in Laemmli Sample Buffer (Bio-Rad #161-0747) to 1 μg/μl. For electrophoresis, 10 μg of each sample were loaded into each lane of a pre-cast Bio-Rad 10% acrylamide gel, and separated proteins were electroblotted overnight at 30V onto 0.45 μm nitrocellulose membrane (Bio-Rad #162-0146). The blots were blocked with NFDM/TBST (5% non-fat dry milk/10 mM Tris-HCl (pH 7.6)/150 mM NaCl/0.05% Tween-20). Primary and secondary antibodies and dilutions are listed in Table 2. Blots were reacted with primary antibody in 5% BSA blocking buffer overnight at 4° C. Secondary antibodies were diluted in 5% NFDM/TBST or 5% BSA and applied for 60 minutes at RT. Reacted blots were covered with HRP-substrate (Amersham #RPN2232) for 1 minute at RT, followed by antigen localization and densitometry using GE ImageQuant software.

TABLE 2
Antibodies for Western Blotting

| | | | Made | |
|---|---|---|---|---|
| Antigen | Manufacturer | Catalog# | in | Dilution |
| 1° Tip60 (N1) | Bethyl | custom | rabbit | 1:1000 |
| 2° goat anti-rabbit IgG HRP | Bio-Rad | 170-6515 | goat | 1:7500 |
| 1° GAPDH | Adv ImmunoChem | 2-RGM2 | mouse | 1:1000 |
| | Inc. | (6C5) | | |
| 2° goat anti-mouse IgG HRP | Bio-Rad | 170-6516 | goat | 1:7500 |
| 1° phosphorylated Atm (pATM) | Santa Cruz | sc-47739 | mouse | 1:1000 |
| 2° goat anti-mouse IgG HRP | Bio-Rad | 170-6516 | goat | 1:4000 |
| 1° phosphorylated ATM (pATM | Novus | NB100- | mouse | 1:1000 |
| serine 1981) | | 306 | | |
| 2° goat anti-mouse IgG HRP | Bio-Rad | 170-6516 | goat | 1:4000 |
| 1° Phosphorylated Chk2Thr68-D12 | Thermo Fisher | MA527988 | rabbit | 1:1000 |
| (monoclonal rabbit IgG1 kappa | Scientific | | | |
| isotope) | | | | |
| 2° goat anti-rabbit IgG HRP | Bio-Rad | 170-6515 | goat | 1:4000 |

Immunostaining & cell counting: On the day before harvest, mice were injected with BrdU as described above. Following removal, hearts were perfused with cardioplegic solution and atria were removed. Ventricles were fixed overnight in fresh 4% paraformaldehyde/PBS, processed through EtOH series and embedded in paraffin. Sections (4 μm thick) mounted on microscope slides were de-waxed, subjected to antigen retrieval (100° C. in 10 mM trisodium citrate pH6.0/0.05% Tween-20 for 20 minutes) followed by 30 minutes' cooling at RT, and blocked with 2% goat serum/0.1% Triton-X-100 in PBS. Primary antibodies were diluted in blocking buffer and applied overnight at 4° C.; secondary antibodies were applied for one hour in the dark; combinations of primary and secondary antibodies employed for each antigen, plus dilutions, are shown in Table 3.

TABLE 3
Antibodies for Immunofluorescent Staining

| Antigen | Manufacturer | Catalog # | Made in | Dilution |
|---|---|---|---|---|
| 1° 5′-bromode- | Abcam | ab6326 | rat | 1:200 |
| oxyuridine (BrdU) | | | | |
| 2° goat anti-rat 488 | Invitrogen | A-11006 | goat | 1:500 |
| 1° phosphohistone H3 | EMD | 05-806 | mouse | 1:200 |
| (pH 3) | Millipore | | | |
| 2° goat anti-mouse 488 | Invitrogen | A-11029 | goat | 1:500 |
| 1° phosphorylated | Novus | NB100-306 | mouse | 1:200 |
| Atm (pAtm) | | | | |
| 2° goat anti-mouse 488 | Invitrogen | A-11029 | goat | 1:500 |
| 1° Ki67 | Invitrogen | 14-5698-82 | rat | 1:250 |
| 2° goat anti-rat 488 | Invitrogen | A-11006 | goat | 1:500 |
| 1° Cre | Millipore | 69050-3 | rabbit | 1:500 |
| 2° goat anti-rabbit 594 | Invitrogen | A-11037 | goat | 1:500 |
| 1° cardiac-Troponin | Neomarker | MS295- | mouse | 1:250 |
| (cTnT) | | P1ABX | | |
| 2° goat anti-mouse 594 | Invitrogen | A-11032 | goat | 1:500 |
| 1° HIF1-α | Novus | NB100- | rabbit | 1:200 |
| | | 479SS | | |
| 2° goat anti-rabbit 488 | Invitrogen | A-11034 | goat | 1:500 |
| 1° CD45 | Invitrogen | 14-0452-82 | rat | 1:200 |
| 2° goat anti-rat 488 | Invitrogen | A-11006 | goat | 1:500 |
| 1° GATA-4 | Cell Signaling | D3A3M | rabbit | 1:100 |
| | Technology | 36966S | | |
| 2° goat anti-rabbit 594 | Invitrogen | A-11037 | goat | 1:500 |

Microscopy was performed on a Nikon Eclipse 50i microscope equipped with a Nikon DSU3 digital camera. It was strongly preferred to identify CMs based on expression of a nuclear marker, such as GATA4, for which an antibody having remarkable specificity and sensitivity was employed (Cell Signaling, Cat. #36966). To quantify the percentage of CMs co-expressing Ki67, BrdU, pH3, or pAtm, these were assessed in the FITC channel, only after confirming the identity of each GATA4-stained CM in the Texas Red channel per the following criteria: (i) staining was verified as nuclear by co-staining with DAPI, (ii) counting was restricted to ovoid/spherical nuclei that were at least 1.5 μm diameter, (iii) only nuclei residing in the myocardium as revealed by auto-fluorescence in the FITC channel were counted, (iv) nuclei located in interstitial spaces, epicardium, or vasculature were ignored. At least 500 GATA4-positive cells were evaluated in each section during scanning at 1,000× magnification. To verify pAtm staining, only nuclei that were at least half-filled with FITC fluorescence were counted; pAtm-positive nuclei were also confirmed as DAPI-positive.

TUNEL labeling & counting: Apoptosis was assessed using the DeadEnd Fluorometric TUNEL System (Promega #G3250) per the manufacturer's instructions. The total number of TUNEL-positive nuclei present in each section was manually counted at 400× magnification. TUNEL signal was counted only if confined to a DAPI-positive nucleus. Nuclei were scored as TUNEL-positive only if at least 50% of the nucleus contained fluorescent signal.

Quantitative Assessment of Myocardial Scarring: Paraffinized hearts were transversely sectioned, in entirety, from apex to base, after which eight 4 μm thick sections from equidistant intervals were placed on microscope slides. The slides were stained with Masson trichrome to quantitatively assess scar size. Briefly, trichrome-stained sections were examined with a Nikon SMZ800 microscope and photographed at 10× magnification using a SPOT Insight camera (Nikon Instruments). MIQuant software was used to quantitate infarct size in sections between the apex and the ligation suture site. Results were expressed as the average percentage of area and midline length around the left ventricle.

Cardiomyocyte (CM) nucleation & ploidy: Cells in the myocardium were separated by perfusing collagenase II (1 mg/ml/PBS) retro-aortically using a Langendorff apparatus, as previously described [14]. Perfused ventricles were isolated, triturated in KB buffer (20 mM KCl/10 mM KH₂PO₄/70 mM K-glutamate/1 mM MgCl₂/25 mM glucose/10 mM β-hydroxybutyric acid/20 mM taurine/0.5 mM EGTA/0.1% albumin/10 mM HEPES [pH 7.4]), and filtered through 250-μm mesh. While homogeneously suspended, the cells were fixed by adding PFA to 2%, followed by immunofluorescent detection of CMs using a primary cTnT antibody (Abcam ab8295; Abcam 1:1,000 overnight at 4° C.) followed by a goat anti-mouse secondary (ThermoFisher A11001, 1:500) and DAPI staining to enable evaluation of nucleation and ploidy. Aliquots of the stained cell suspension were spread across microscope slides, followed by application of a coverslip and microscopic examination at 200× magnification. CM nucleation (mono-, bi, tri-, tetra-) was determined by evaluating a minimum of 200 CMs per heart. Nuclear ploidy was estimated from photomicrographs subjected to ImageJ analysis to determine the relative concentration of DNA in each nucleus as inferred by pixilation density within each DAPI-stained (blue channel).

Data analysis and statistics: All determinations were performed by blinded observers. Data are reported as means±SEM. Global data encompassing the P7 thru P31 timepoints were analyzed using a two-way repeated measures ANOVA (time and genotype) analysis to determine whether effects encompassed both time and genotype (i.e. time-genotype interaction). If a global test indicated an effect, post hoc contrasts between baseline and subsequent timepoints within an experimental group was compared using the Dunnett's multiple comparison t test. To assess differences between genotypes at each timepoint, Student's t test with the Bonferroni correction was used. All other data were compared by an unpaired, two-tailed Student's t test.

### Results:

Depleting Tip60 while Avoiding Postnatal Lethality

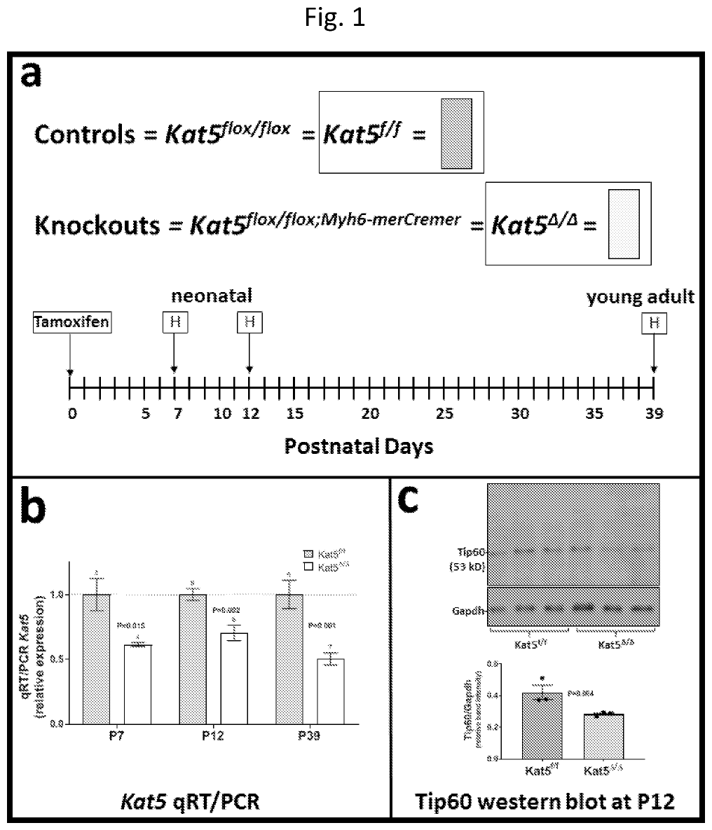

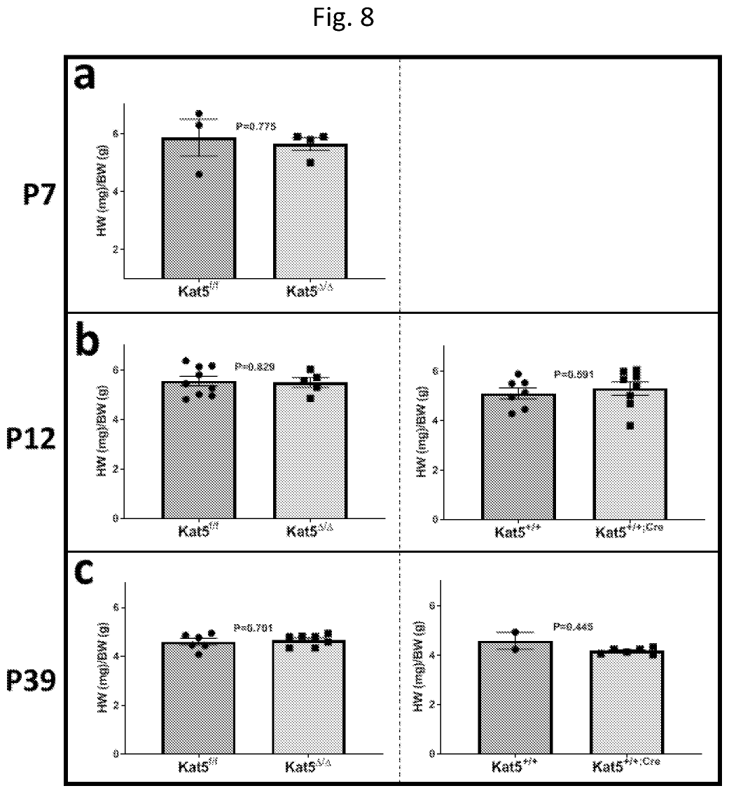

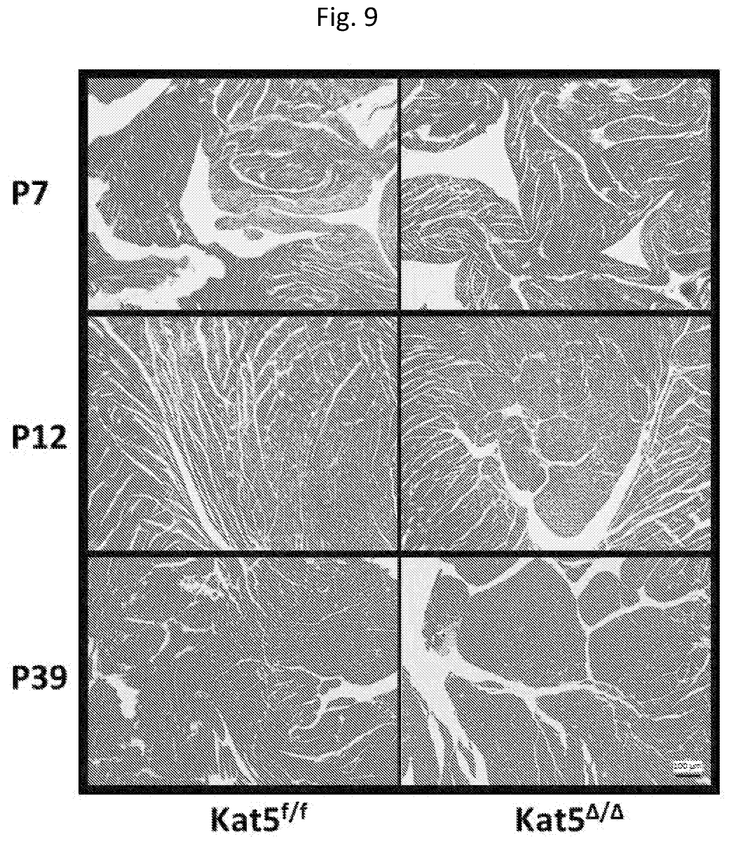

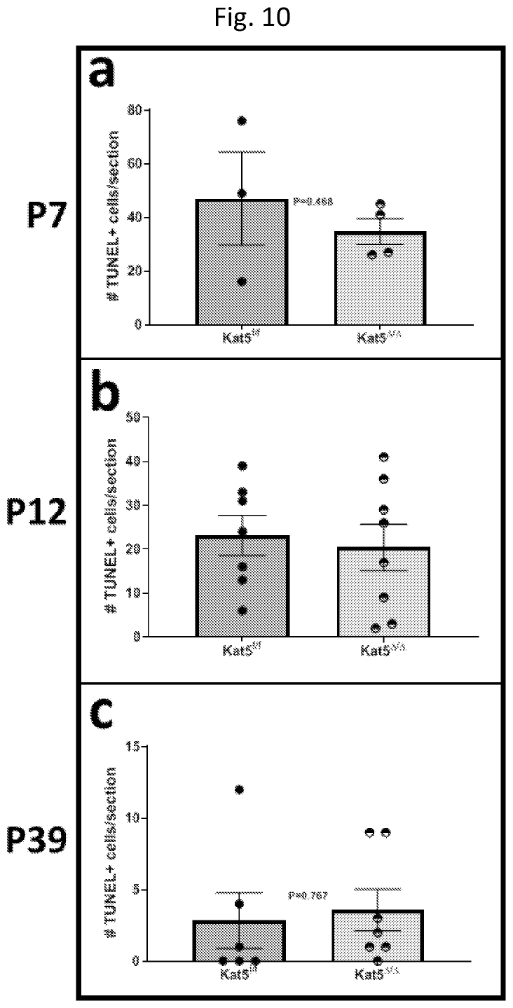

The objective of this study was to assess the effects of disrupting the Kat5 gene, which encodes Tip60, from postnatal CMs beginning at the time of birth (P0). It was anticipated that Tip60 depletion would inhibit the DDR as indicated by reduced levels of pAtm, thereby, in accord with previous findings [11], extending the period of CM proliferation in the neonatal mammalian heart. Because over-depletion of Tip60 is lethal to cells [4] including CMs [5], we decided to conditionally activate the Myh6-cre transgene by injecting tamoxifen into control (Kat5^{flox/flox}, hereafter Kat5^{f/f}) and experimental (Kat5^{flox/flox;Myh6-merCremer} hereafter Kat5^{Δ/Δ}) mice at only one timepoint, postnatal day 0 (P0). As depicted in FIG. 1a, hearts of mice bearing these genotypes were evaluated at postnatal days P7, P12, and P39, the latter to assess the effect of neonatal Tip60 depletion at an early adult stage. As described below, this regimen produced a phenotype that was mild in comparison with the phenotype we observed after injecting adult mice with tamoxifen on multiple days (see Example 2 below); unfortunately, however, multiple injections of tamoxifen during early neonatal stages caused lethality. The single dose regimen significantly depleted Kat5 mRNA in Kat5^{Δ/Δ} hearts at each stage (FIG. 1b), and, western blotting at P12 indicated that Tip60 protein was similarly depleted (FIG. 1c). Importantly, the absence of altered cardiac mass (FIG. 8), fibrosis (FIG. 9), and cell death (FIG. 10) in Tip60-depleted hearts indicated that this regimen did not cause untoward effects during neonatal heart development.

### Tip60 Depletion Reduces Atm Phosphorylation

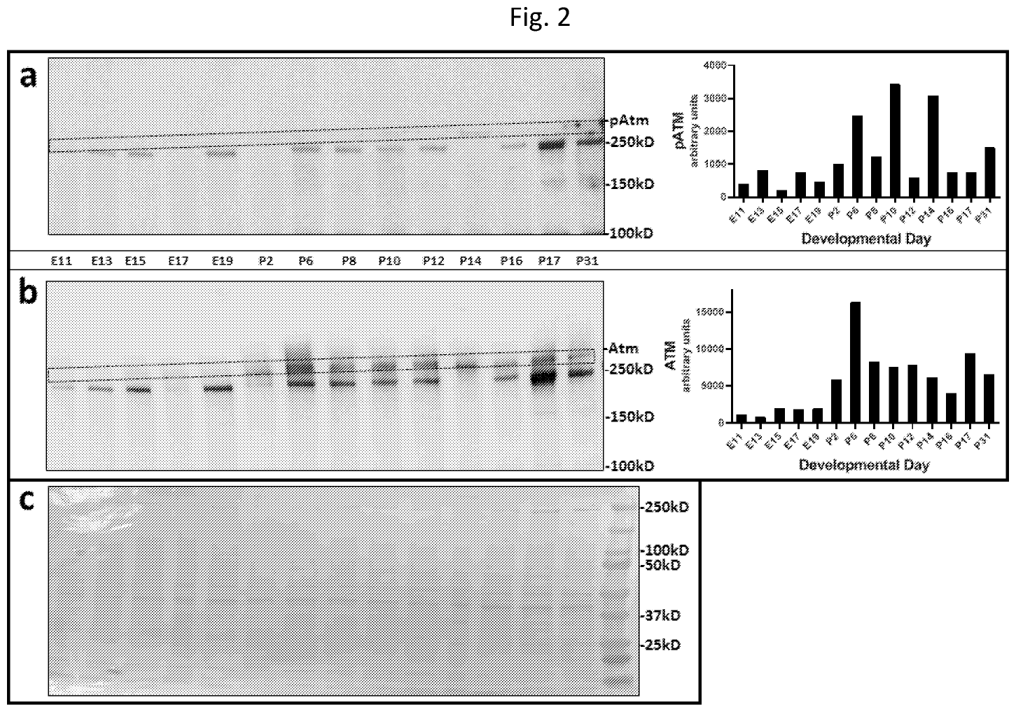

It was recently reported that Atm phosphorylation in CMs at early neonatal stages activates the DNA Damage Response (DDR), which culminates in CM proliferative senescence [11]. It was therefore of interest to determine the developmental pattern of pAtm and bulk Atm expression at successive stages of heart development in wild-type murine hearts. For this purpose, embryonic and adult hearts collected at the developmental stages shown in FIG. 2 were subjected to western blotting. Despite challenges due to low levels of this large phosphorylated protein (˜350 kD) in a background dominated by non-CMs, this revealed a consistent trend toward increased pAtm levels beginning in the early neonatal period (P2; FIG. 2a), a pattern is consistent with previous findings [11]. These observations were accompanied by readily detectable increases in non-phosphorylated (bulk) Atm early in the postnatal period (FIG. 2b).

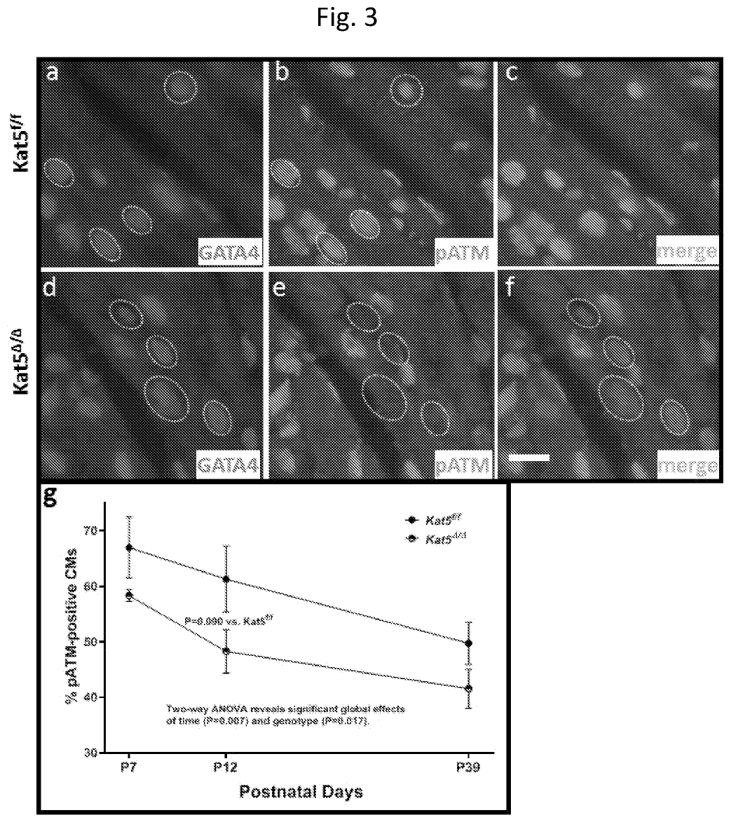

Because CMs constitute a minority cell type in the murine heart [15, 16], samples evaluated by western blotting provide a relatively crude estimate of CM protein content. Therefore, to assess the effect of Tip60 depletion on the percentage of CMs expressing pAtm, immunofluorescent microscopy was employed. These assessments we performed adhering to rigorous ground rules detailed in Materials and Methods, wherein quantitation was restricted to the enumeration of CM nuclei identified by robust GATA4 staining (FIG. 3a,d) that exhibited pAtm signal in at least 50% of the nuclear area (FIG. 3b,e). This revealed that in comparison with the percentage of pAtm-positive CMs in Kat5^{f/f} control hearts, which declined between stages P7 and P39, the percentages in Tip60-depleted CMs were reduced at all stages. (FIG. 3g). This result suggests that depletion of Tip60 in CMs at early neonatal stages inhibits the DDR.

### Genes Encoding Meis1 and p27 are Decreased in Tip60-Depleted Hearts

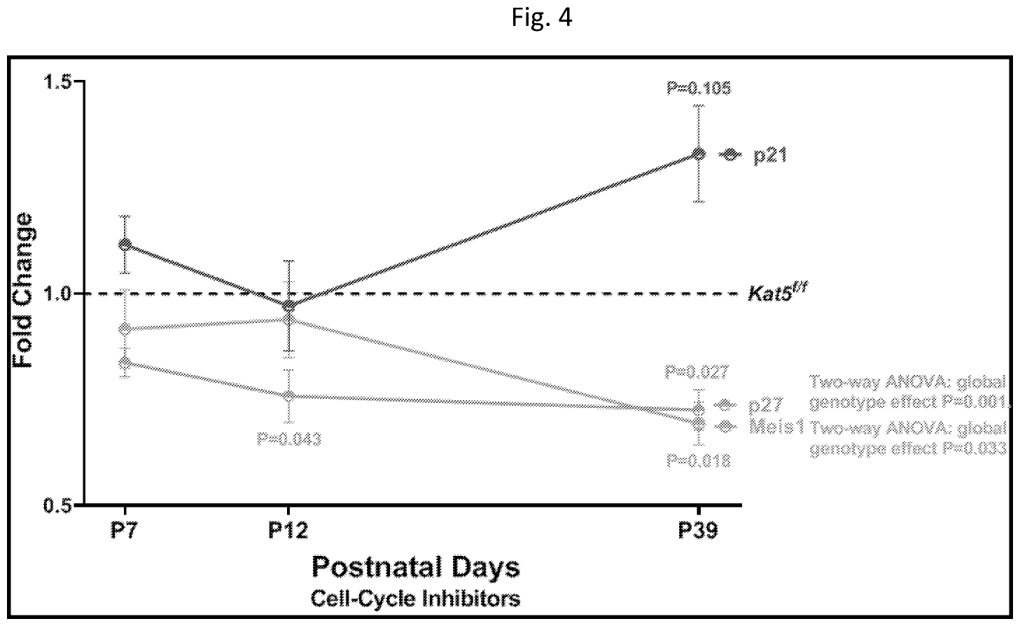

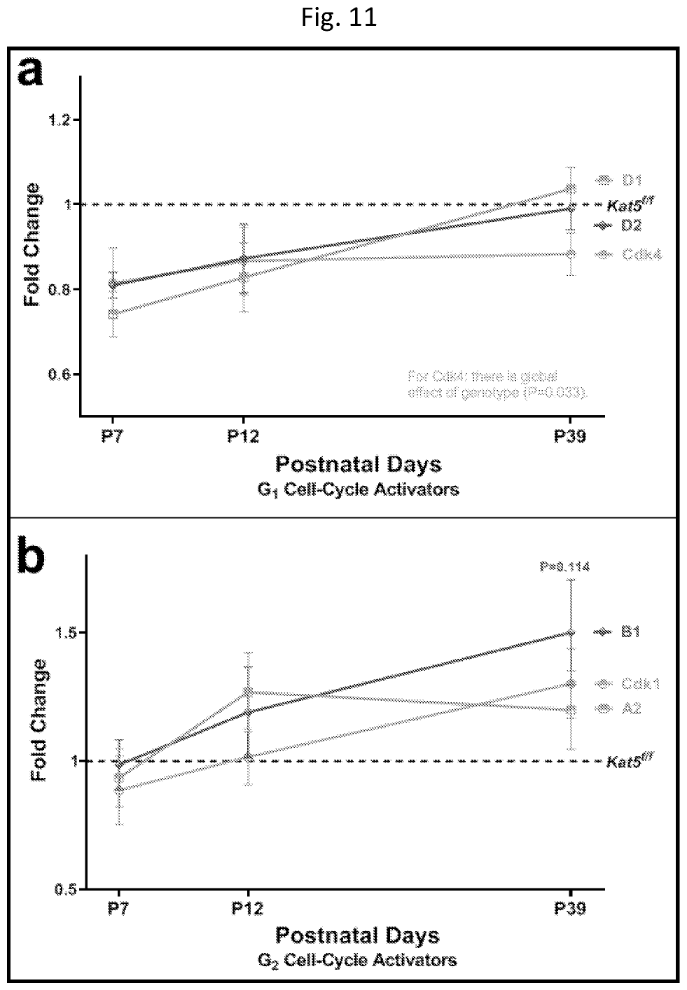

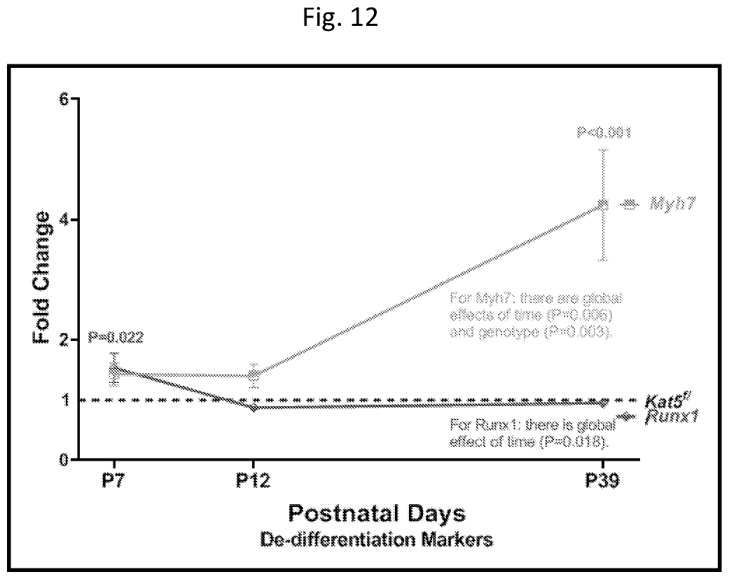

In accord with our hypothesis, depletion of Tip60 in Kat5^{Δ/Δ} hearts, as a consequence of reducing pAtm, should inhibit, or at minimum delay, the onset of CM replicative senescence. To address these possibilities, the assessments described in FIGS. 4-6 were performed, beginning with qPCR determinations to assess the effect of Tip60 depletion on the expression of genes that regulate the cell cycle. The effect on genes that activate the cell cycle are shown in FIG. 11, indicating that while genes that activate early (G₁) cell cycle phases (cyclins D1, D2 and Cdk4) were not activated and perhaps even depressed (FIG. 11a), genes activating late (G₂) cell cycle phases (Cyclins A2, B1) exhibited increases in Tip60-depleted hearts beginning at P12, continuing to increase at P39 when Cdk1 was also increased (FIG. 11b). While supporting a trend, these changes did not achieve statistical significance, likely due to the presence of non-CMs in the whole heart samples taken for qPCR. We also evaluated the expression of de-differentiation markers that become up-regulated prefatory to CM cell cycle activation [17-19]; among these, Myh7 expression was increased at all stages, culminating in a highly significant 4-fold increase by P39 (FIG. 12).

The most remarkable response to Tip60 depletion was the reduced expression of genes encoding cell cycle inhibitor proteins Meis1 and p27. As shown in FIG. 4, reduced expression of Meis1 and p27 was noted at P7, which became statistically significant for p27 at P12 and for both inhibitors at P39. Curiously, inhibited expression of Meis1 and p27 at P39 was accompanied by increased expression of the gene encoding p21, which also inhibits the CM cell cycle.

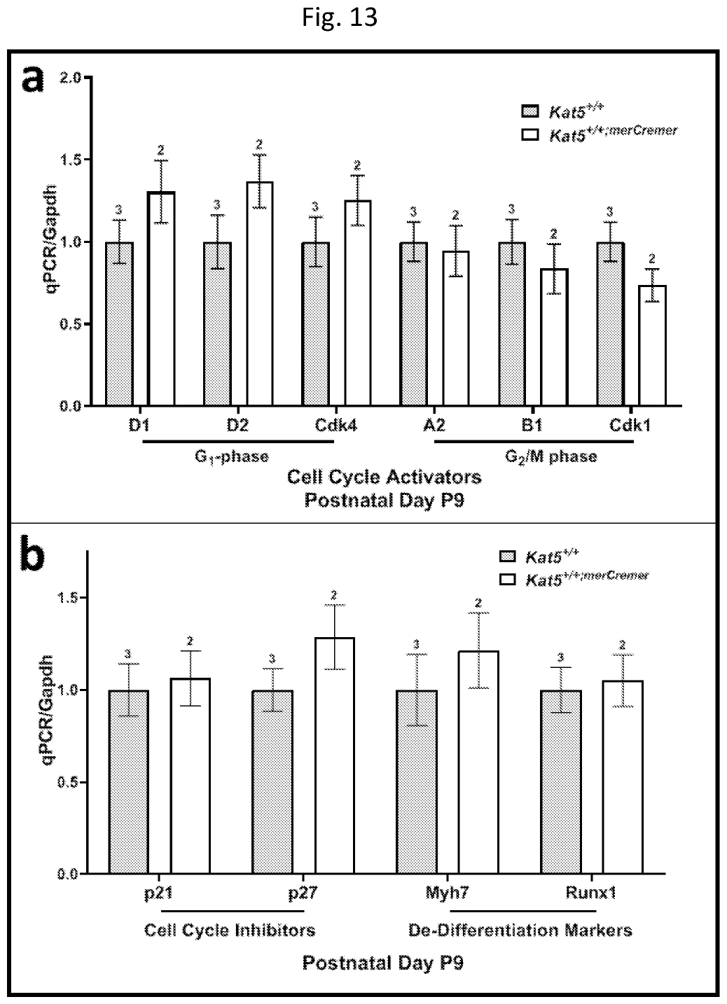

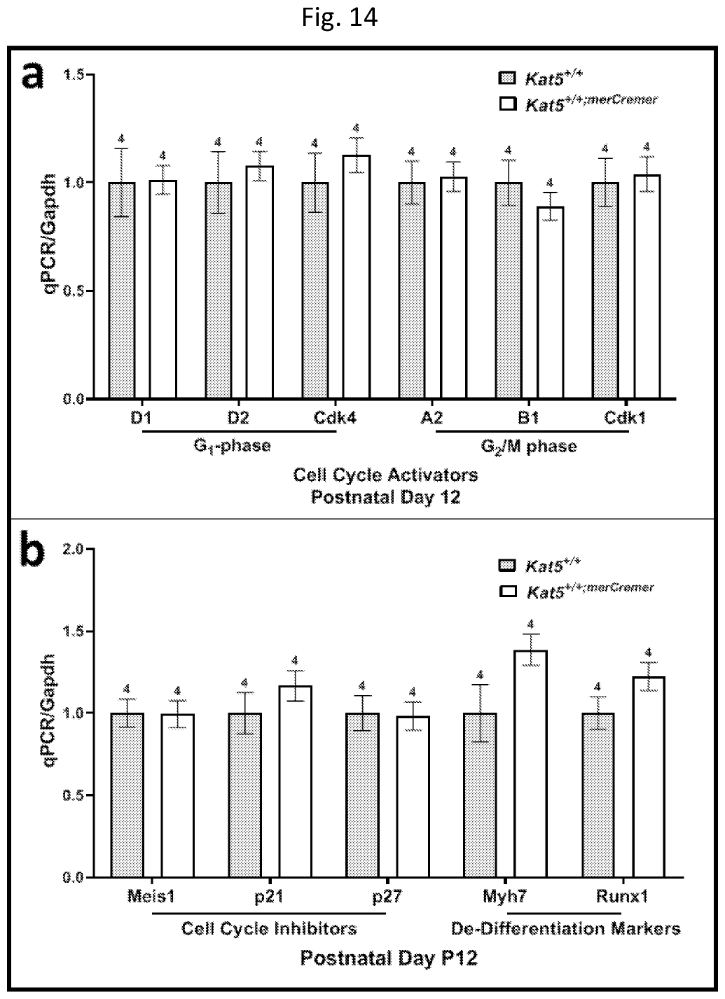

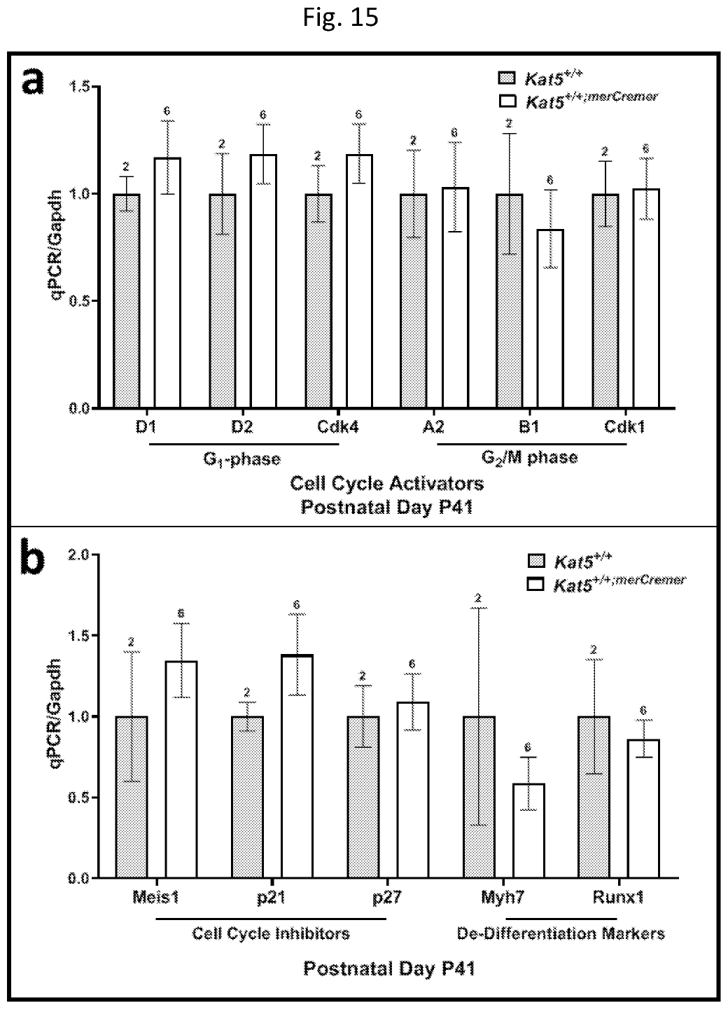

Several reports have described cardiac dysfunction that may be caused by off-target effects of the Myh6-driven merCremer-recombinase transgene employed in this study [20-23]. To assess whether Cre alone may have affected the gene expressions described above, we evaluated its effect in a LoxP-free genetic background by comparing the expression of these genes in wild-type (Kat5^{+/+}) control hearts versus Kat5^{+/+;Myh6-merCremer} hearts that express Cre-recombinase. As shown in FIGS. 13-15, there was no evidence indicating that the changes in gene expression attributed to LoxP-mediated Tip60 depletion were caused by off-target effects of Cre.

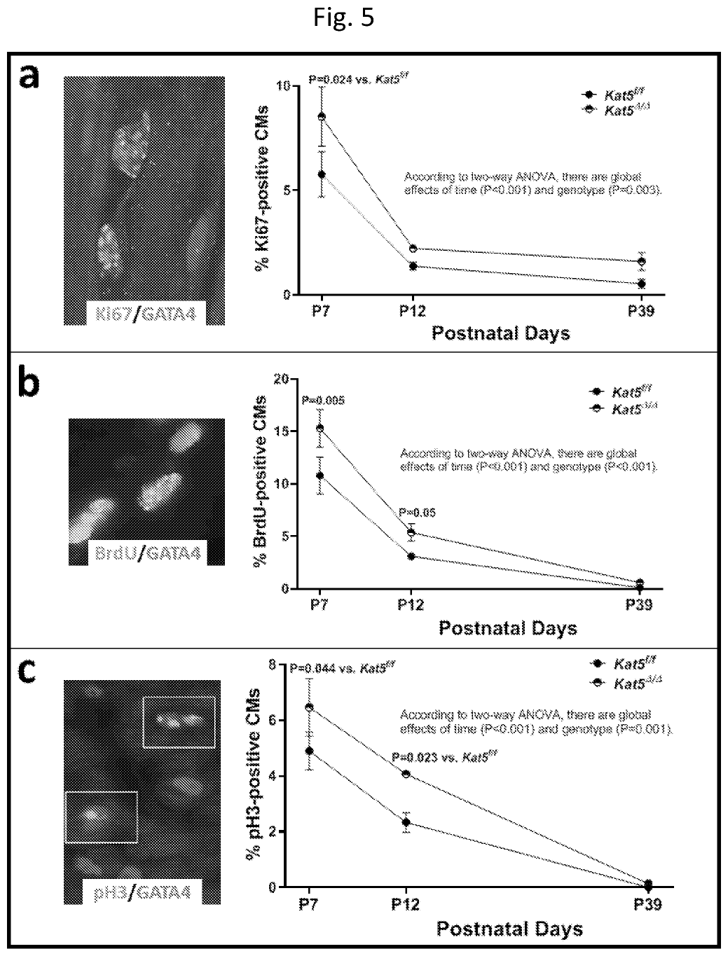

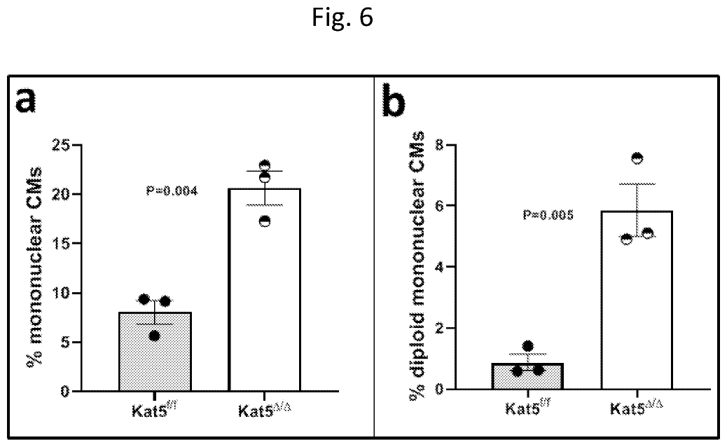

In summary, qPCR revealed that maturing hearts containing CMs depleted of Tip60 have significantly reduced levels of mRNAs encoding the cell cycle inhibitors Meis1 and p27 (FIG. 4), concomitant with significantly increased levels of mRNA encoding the de-differentiation marker Myh7 (FIG. 12). Because these circumstances suggested that Tip60 depletion promotes CM proliferation, the determinations shown in FIGS. 5 and 6 were performed.

### Cell Cycle Activation Markers Ki67, BrdU and pH3 are Up-Regulated in Tip60-Depleted Cardiomyocytes.

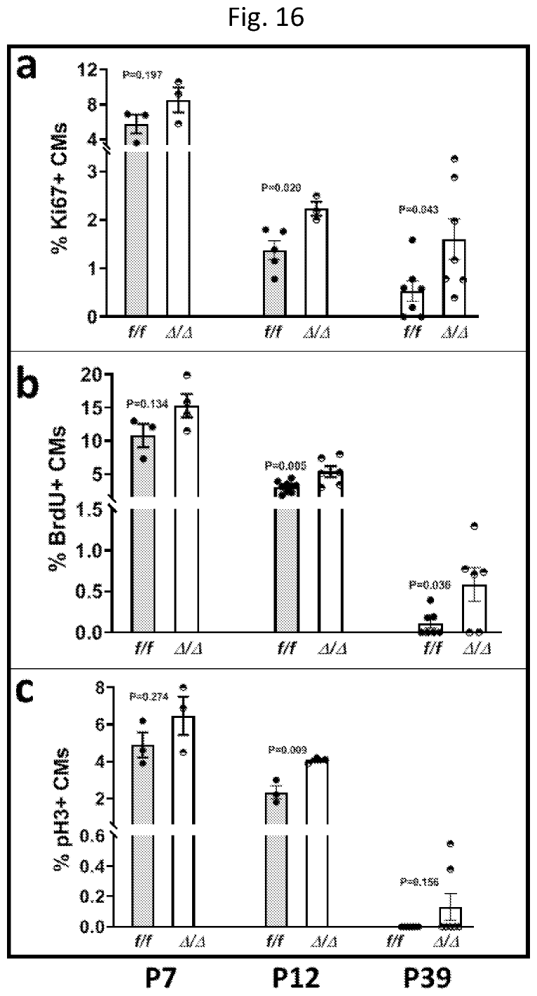

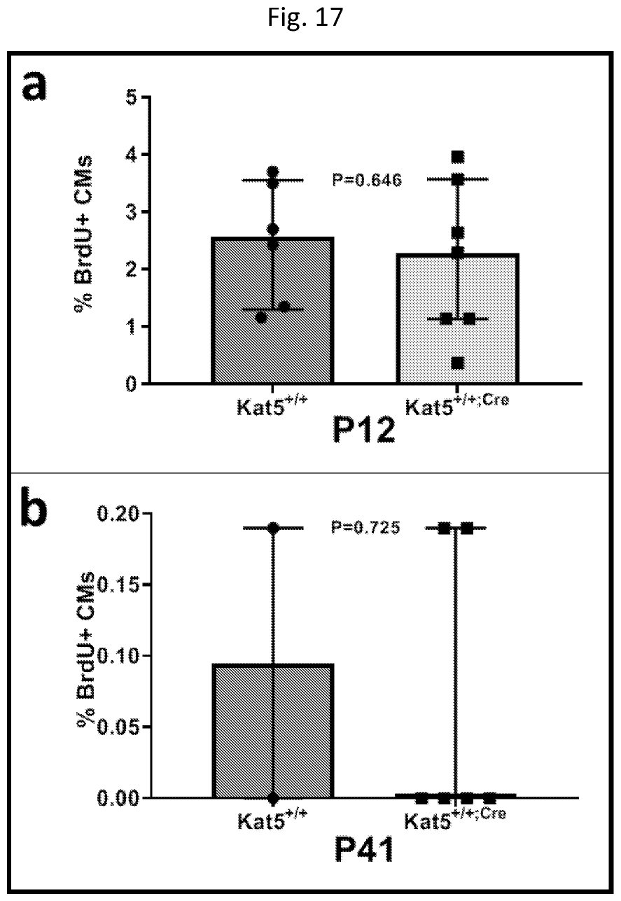

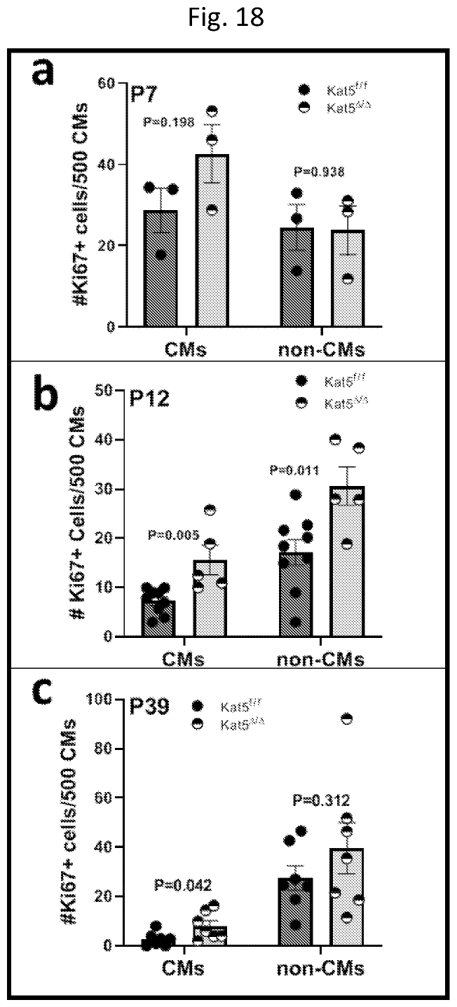

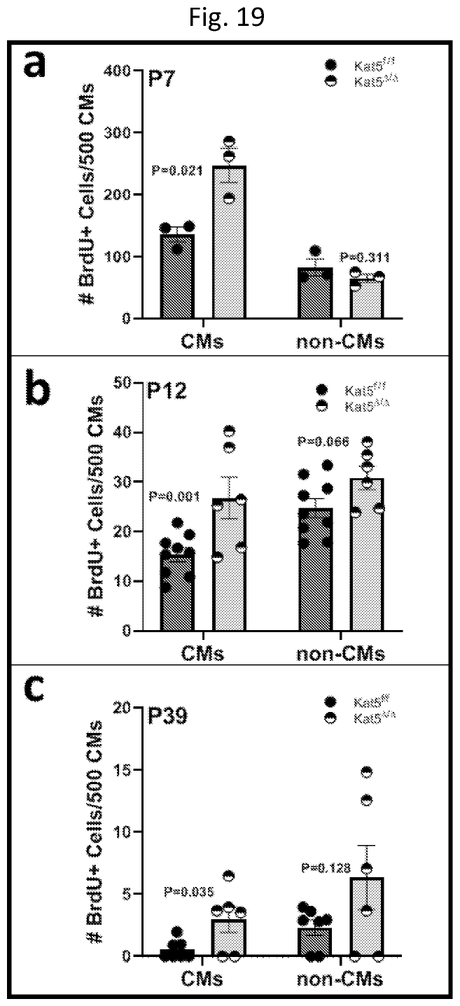

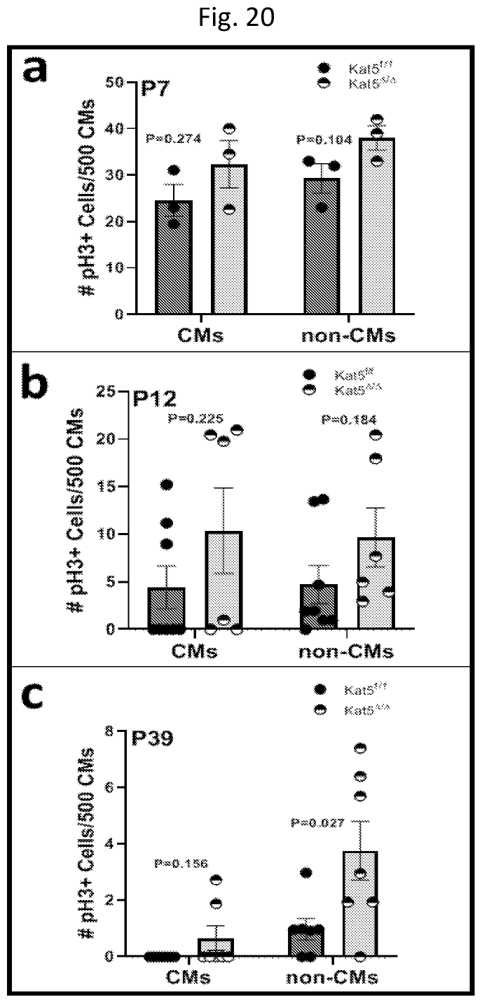

To assess whether the respectively increased and decreased expression of cell cycle activators and inhibitors was accompanied by increased percentages of CMs exhibiting markers of cell cycle transit, the immunohistochemical determinations summarized in FIG. 5 were performed. For these assessments, sections of ventricular myocardium from control and Tip60-depleted hearts at developmental stages P7, P12 and P39 were immunostained for Ki67 (FIG. 5a), 5′-bromodeoxyuridine (BrdU; FIG. 5b), and phosphorylated histone H3 (pH3; FIG. 5c), markers that respectively identify cells in all cell cycle stages, S-phase, and early M-phase. In accord with our preference to probe nuclear antigens, GATA4 was employed to verify CM identity. Blinded quantitation revealed that Tip60 depletion initiated on P0 increased the population of CMs exhibiting cell cycle activation; specifically, Tip60-depleted CMs exhibited the following ranges of percentage increase among the three cell cycle markers monitored at each stage: 32-48% at P7, 62-72% at P12, and 300-532% at P39. With the exception of the 32-48% increases observed on P7, all increases were statistically significant; bar graphs showing individual data points for these determinations are shown in FIG. 16. Controls wherein BrdU incorporation in Kat5^{+/+;Myh6-merCremer} and wild-type (Kat5^{+/+}) CMs was compared at P12 showed that increased cell cycle transit was not caused by Cre alone (FIG. 17). Surprisingly, despite specific depletion of Tip60 in CMs, these findings were accompanied by increased numbers of Ki67-, BrdU- and pH3-positive non-CMs at P12 and P39 as respectively shown in FIGS. 18-20.

Taken together, the findings described in FIGS. 4-5 and their supporting data indicate that conditional depletion of Tip60 activates the cell cycle in postnatal CMs. Despite these increases, proliferative senescence nonetheless ensued as indicated by decreasing cell cycle activation in both genotypes at successive postnatal stages. Nonetheless, increased levels of cell cycle transit in CMs remained in Tip60-depleted hearts at P39 (FIG. 5).

### Mononucleated Diploid Cardiomyocytes are Increased in Tip60-Depleted Hearts

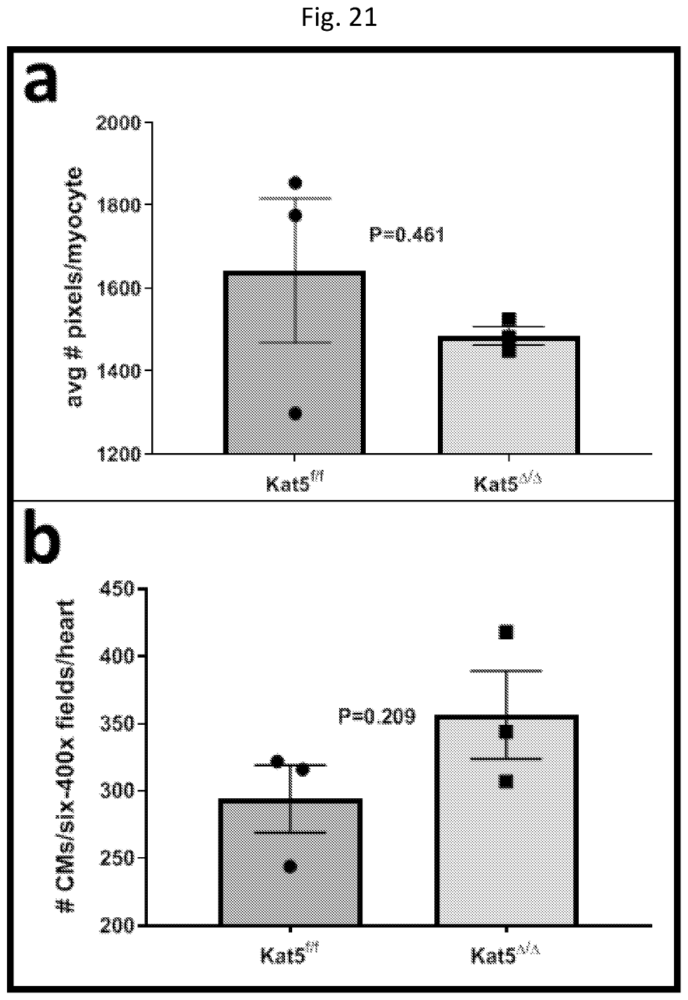

The foregoing determinations did not address the important question of whether, in addition to activating the cell cycle, Tip60 depletion permitted postnatal CMs to undergo bona fide proliferation, as defined by increased numbers of CMs that are both mononuclear and diploid [14]. Normally, during the onset of replicative senescence in the murine heart beginning at ˜P7, endomitosis of cycling CMs results in a preponderance of binucleated diploid CMs by adult stages. It was therefore of interest to ascertain whether Tip60 depletion and the consequent activation of cell cycle markers described in FIGS. 4-5 resulted in increased percentages of mononucleated diploid CMs (MNDCMs), indicative of cytokinesis and complete CM division. To address this question, single-cell suspensions of ventricular CMs were isolated from Kat5^{f/f} and Kat5^{Δ/Δ} hearts at P12, and analyzed for multinuclearity and ploidy. As shown in FIG. 6, the percentage of mononucleated CMs was increased from ˜8% in control hearts to ˜20% in Tip60-depleted (Kat5^{Δ/Δ}) hearts, a ˜2.5-fold increase (FIG. 6a). Importantly, the percentage of mononuclear CMs containing diploid nuclei was also increased in Tip60-depleted hearts, by nearly 7-fold (from 0.9 to 5.9%; FIG. 6b). The increase in MNDCMs is consistent with a trend toward smaller and increased numbers of transversely-sectioned CMs that we observed in wheat germ agglutinin (WGA)-stained sections of Tip60-depleted hearts at P12 (FIG. 21). These results suggest that reduction of Tip60 levels in neonatal CMs permits the generation of MNDCMs, cells that have been shown to possess pro-regenerative potential [14].

Tip60 Depletion Improves Heart Regeneration after Myocardial Infarction.

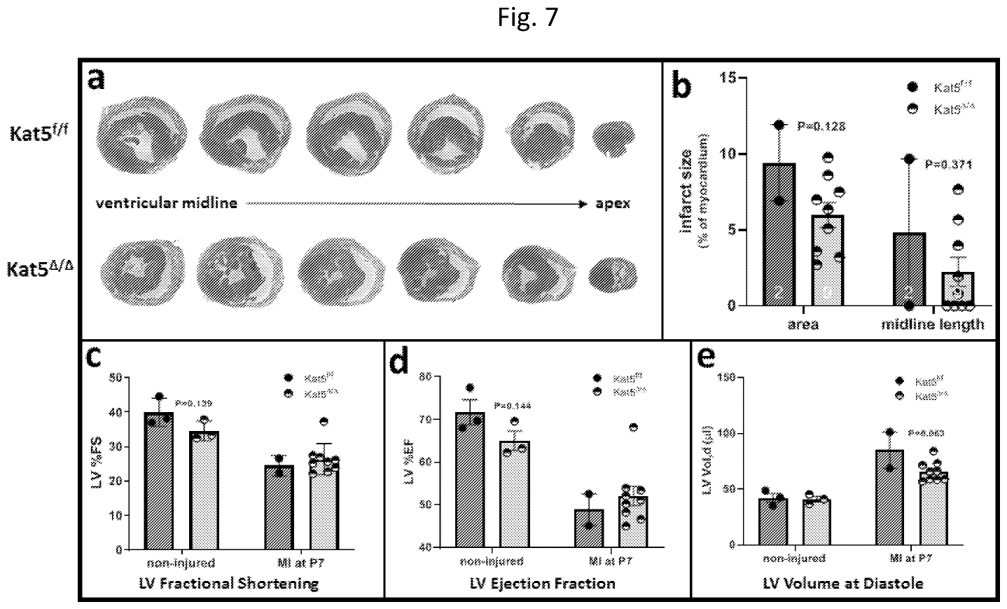

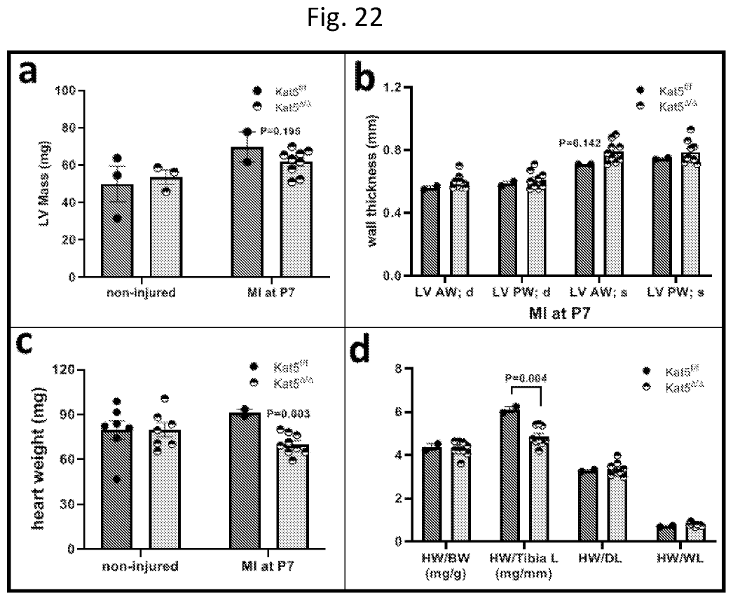

Because the above results (FIG. 6, FIG. 21) correlating Tip60 depletion with increased numbers of MNDCMs at P12 suggested that Tip60-depletion might improve regeneration after myocardial infarction (MI), the determination described in FIG. 7 was performed. In this experiment, Kat5^{f/f} and Kat5^{Δ/Δ} hearts that had been treated with tamoxifen on P0 were infarcted on neonatal day P7 by permanently ligating the left main coronary artery, followed by histologic and echocardiographic assessments at P39 to evaluate scar formation and cardiac function. The extent of scarring in transverse segments of the left ventricle below the midpoint was assessed by Masson trichrome staining (FIG. 7a), which when quantitatively evaluated revealed a trend toward reduced scarring in Tip60-depleted hearts (FIG. 7b). This observation was accompanied by subtly increased fractional shortening (FS; FIG. 7c) and ejection fraction (EF; FIG. 7d), which correlated with remarkably reduced ventricular volume during diastole (FIG. 7e); although subtle, these increases in FS and EF were notable because non-injured Tip60-depleted hearts exhibited decreased function at P39 (FIGS. 7d,e). Echocardiography also revealed possible thickening of the left ventricular walls in infarcted Kat5^{Δ/Δ} hearts during systole (FIG. 22b), combined with reductions in left ventricular mass (FIG. 22a) and overall heart weight (FIG. 22c).

### DISCUSSION

Work on transformed cells has shown that Tip60 is an acetylase that has multiple functions including cell cycle inhibition [8, 24], induction of apoptosis [6, 25], and induction of the DDR [10, 26] via acetylation of Atm. These functions are not mutually exclusive. Although Tip60 is expressed in all tissues examined, including the heart [3, 27], its in vivo functions remain unclear. Because activation of Atm was recently implicated as the inducer of CM proliferative senescence in the neonatal heart [11], it was of interest to ascertain whether Tip60 may induce this process. Therefore we examined the effects of conditionally depleting Tip60 in CMs, at the time of birth and in a fashion that avoids CM pathogenesis [5], on Atm phosphorylation and CM proliferation. Our findings, which indicate that Tip60 depletion reduces pAtm (FIG. 3) while increasing CM cell cycle activation (FIG. 5) and the percentage of MNDCMs (FIG. 6), suggest that Tip60 induces CM proliferative senescence in the maturing heart via Atm signaling; this possibility is consistent with previous findings made in this laboratory [3, 5]. Although Tip60 depletion diminished but did not prevent CM replicative senescence (FIG. 5), this may represent a relatively modest phenotype caused by the requirement to prevent exhaustive Tip60 depletion, or it may reflect intransigence of the postmitotic CM state as previously shown by the inability of inhibiting oxidative damage (which induces the DDR) to prevent CM senescence [11].

Two recent reports from the same laboratory support our contention that Tip60 inhibits CM proliferation. The first report [28] demonstrated that sustained activation of the de-acetylase Sirt1 increases CM proliferation; although Sirt1's targets were not considered, it is interesting that Tip60, which possesses 15 acetylation sites among which lysines K327 and K357 must be auto-acetylated to maintain its HAT activity, is a substrate of Sirt1 [29]. More recently [30], these investigators reported that Sirt1 targets p21, de-acetylation of which subjects it to ubiquitination and degradation, permitting CM proliferation in the adult heart.

Among the molecular responses to Tip60 depletion we interrogated, the most promising in terms of permitting CM cell cycle activation have been the observed decreases in Meis1 and p27 mRNAs (FIG. 4); to our knowledge, however, a connection between Tip60 and either of these cell cycle inhibitors has not been reported. The trends toward differential effects of Tip60 depletion on expression of the G₁-phase and G₂-phase cell cycle activators, wherein G₁-phase activators were reduced (FIG. 11a) as G₂-phase activators increased (FIG. 11b), was not anticipated. Its interesting to consider this result vis-a-vis findings that over-expression of G₂-phase activators strongly increases proliferation in cultured P7 CMs, which subsequently die unless G₁-phase activators are co-expressed [31]. Although the authors reasoned that CM death induced by G₂ activators resulted from premature entry into M-phase, a possibility consistent with other findings [32] discussed below, how G₁ activators might prevent CM death while permitting continued proliferation remains unclear. In any event, the conditions employed to deplete Tip60 in this study revealed no evidence of CM death at any stage (FIG. 10). Finally, it is interesting to consider the up-regulation of G₂-phase activators in light of previous findings implying that Tip60 blocks the cell cycle of kidney [24] and HeLa [33] cells in G₂-phase, raising the possibility that depletion of Tip60 releases a cohort of CMs that had been suspended in G₂-phase of the cell cycle.

It has been speculated that the virtual absence of CM proliferation in the adult myocardium is largely due to multiple layers of inhibition that become established during mid to late neonatal stages [2, 34]. To date, various proteins capable of inhibiting CM proliferation have been identified, include retinoblastoma [35, 36], Meis1 [37] and Meis2 [36], components of the Hippo pathway[38], and glycogen synthase kinase (Gsk) [32, 39]. The goal of regenerating the myocardium by permitting resumption of existing CM proliferation would be most efficiently fulfilled by targeting a single protein that potently blocks this process. As recently reviewed [40], one potently inhibitory candidate is glycogen synthase kinase (Gsk), based on findings that ablation of Gskβ in fetal CMs [39] causes lethality due to unchecked CM proliferation, and findings that co-ablation of Gskα and Gskβ in the adult myocardium causes mitotic catastrophe of CMs [32]. The remarkable effects of Gsk depletion in CMs are relevant to our findings because Gsk, a well-established component of the Akt signaling pathway, directly phosphorylates Tip60 at serines 86 and 90, which is required to maintain its acetyltransferase activity [41-45]. Taken together, these findings support the compelling possibility that a strongly inhibitory Gsk→Tip60 axis exists in CMs, inhibition of which releases CMs from replicative arrest. Again, although depletion of Tip60 in this study only modestly increased CM proliferation without preventing replicative senescence, the extent of its depletion was deliberately limited to avoid CM lethality. It will be interesting to assess the effects of extensive albeit transient depletion of Gsk and/or Tip60 in CMs.

### REFERENCES

[1] H. Sadek, E. N. Olson, Toward the Goal of Human Heart Regeneration, Cell Stem Cell 26(1) (2020) 7-16.
[2] F. X. Galdos, Y. Guo, S. L. Paige, N.J. VanDusen, S. M. Wu, W. T. Pu, Cardiac Regeneration: Lessons From Development, Circ Res 120(6) (2017) 941-959.
[3] J. B. Fisher, M. S. Kim, S. Blinka, Z. D. Ge, T. Wan, C. Duris, D. Christian, K. Twaroski, P. North, J. Auchampach, J. Lough, Stress-induced cell-cycle activation in Tip60 haploinsufficient adult cardiomyocytes, PLoS One 7(2) (2012) e31569.
[4] Y. Hu, J. B. Fisher, S. Koprowski, D. McAllister, M. S. Kim, J. Lough, Homozygous disruption of the Tip60 gene causes early embryonic lethality, Dev Dyn 238(11) (2009) 2912-21.
[5] J. B. Fisher, A. Horst, T. Wan, M. S. Kim, J. Auchampach, J. Lough, Depletion of Tip60 from In Vivo Cardiomyocytes Increases Myocyte Density, Followed by Cardiac Dysfunction, Myocyte Fallout and Lethality, PLoS One 11(10) (2016) e0164855.
[6] S. M. Sykes, H. S. Mellert, M. A. Holbert, K. Li, R. Marmorstein, W. S. Lane, S. B. McMahon, Acetylation of the p53 DNA-binding domain regulates apoptosis induction, Mol Cell 24(6) (2006) 841-51.
[7] Y. Tang, W. Zhao, Y. Chen, Y. Zhao, W. Gu, Acetylation is indispensable for p53 activation, Cell 133(4) (2008) 612-26.
[8] M. Squatrito, C. Gorrini, B. Amati, Tip60 in DNA damage response and growth control: many tricks in one HAT, Trends Cell Biol 16(9) (2006) 433-42.
[9] Y. Sun, X. Jiang, B. D. Price, Tip60: connecting chromatin to DNA damage signaling, Cell Cycle 9(5) (2010) 930-6.
[10] Y. Sun, X. Jiang, Y. Xu, M. K. Ayrapetov, L. A. Moreau, J. R. Whetstine, B. D. Price, Histone H3 methylation links DNA damage detection to activation of the tumour suppressor Tip60, Nat Cell Biol 11(11) (2009) 1376-82.
[11] B. N. Puente, W. Kimura, S. A. Muralidhar, J. Moon, J. F. Amatruda, K. L. Phelps, D. Grinsfelder, B. A. Rothermel, R. Chen, J. A. Garcia, C. X. Santos, S. Thet, E. Mori, M. T. Kinter, P. M. Rindler, S. Zacchigna, S. Mukherjee, D. J. Chen, A. I. Mahmoud, M. Giacca, P. S. Rabinovitch, A. Aroumougame, A. M. Shah, L. I. Szweda, H. A. Sadek, The oxygen-rich postnatal environment induces cardiomyocyte cell-cycle arrest through DNA damage response, Cell 157(3) (2014) 565-79.
[12] Y. Nakada, D. C. Canseco, S. Thet, S. Abdisalaam, A. Asaithamby, C. X. Santos, A. M. Shah, H. Zhang, J. E. Faber, M. T. Kinter, L. I. Szweda, C. Xing, Z. Hu, R. J. Deberardinis, G. Schiattarella, J. A. Hill, O. Oz, Z. Lu, C. C. Zhang, W. Kimura, H. A. Sadek, Hypoxia induces heart regeneration in adult mice, Nature 541(7636) (2017) 222-227.
[13] D. S. Sohal, M. Nghiem, M. A. Crackower, S. A. Witt, T. R. Kimball, K. M. Tymitz, J. M. Penninger, J. D. Molkentin, Temporally regulated and tissue-specific gene manipulations in the adult and embryonic heart using a tamoxifen-inducible Cre protein, Circ Res 89(1) (2001) 20-5.
[14] M. Patterson, L. Barske, B. Van Handel, C. D. Rau, P. Gan, A. Sharma, S. Parikh, M. Denholtz, Y. Huang, Y. Yamaguchi, H. Shen, H. Allayee, J. G. Crump, T. I. Force, C. L. Lien, T. Makita, A. J. Lusis, S. R. Kumar, H. M. Sucov, Frequency of mononuclear diploid cardiomyocytes underlies natural variation in heart regeneration, Nat Genet 49(9) (2017) 1346-1353.
[15] I. Banerjee, J. W. Fuseler, R. L. Price, T. K. Borg, T. A. Baudino, Determination of cell types and numbers during cardiac development in the neonatal and adult rat and mouse, Am J Physiol Heart Circ Physiol 293(3) (2007) H1883-91.
[16] A. R. Pinto, A. Ilinykh, M. J. Ivey, J. T. Kuwabara, M. L. D'Antoni, R. Debuque, A. Chandran, L. Wang, K. Arora, N. A. Rosenthal, M. D. Tallquist, Revisiting Cardiac Cellular Composition, Circ Res 118(3) (2016) 400-9.
[17] G. D'Uva, A. Aharonov, M. Lauriola, D. Kain, Y. Yahalom-Ronen, S. Carvalho, K. Weisinger, E. Bassat, D. Rajchman, O. Yifa, M. Lysenko, T. Konfino, J. Hegesh, O. Brenner, M. Neeman, Y. Yarden, J. Leor, R. Sarig, R. P. Harvey, E. Tzahor, ERBB2 triggers mammalian heart regeneration by promoting cardiomyocyte dedifferentiation and proliferation, Nat Cell Biol 17(5) (2015) 627-38.
[18] S. Ikeda, W. Mizushima, S. Sciarretta, M. Abdellatif, P. Zhai, R. Mukai, N. Fefelova, S. I. Oka, M. Nakamura, D. P. Del Re, I. Farrance, J. Y. Park, B. Tian, L. H. Xie, M. Kumar, C. P. Hsu, S. Sadayappan, H. Shimokawa, D. S. Lim, J. Sadoshima, Hippo Deficiency Leads to Cardiac Dysfunction Accompanied by Cardiomyocyte Dedifferentiation During Pressure Overload, Circ Res 124(2) (2019) 292-305.
[19] K. Gabisonia, G. Prosdocimo, G. D. Aquaro, L. Carlucci, L. Zentilin, I. Secco, H. Ali, L. Braga, N. Gorgodze, F. Bernini, S. Burchielli, C. Collesi, L. Zandona, G. Sinagra, M. Piacenti, S. Zacchigna, R. Bussani, F. A. Recchia, M. Giacca, MicroRNA therapy stimulates uncontrolled cardiac repair after myocardial infarction in pigs, Nature 569(7756) (2019) 418-422.
[20] N. Koitabashi, D. Bedja, A. L. Zaiman, Y. M. Pinto, M. Zhang, K. L. Gabrielson, E. Takimoto, D. A. Kass, Avoidance of transient cardiomyopathy in cardiomyocyte-targeted tamoxifen-induced MerCreMer gene deletion models, Circ Res 105(1) (2009) 12-5.
[21] M. E. Hall, G. Smith, J. E. Hall, D. E. Stec, Systolic dysfunction in cardiac-specific ligand-inducible MerCreMer transgenic mice, Am J Physiol Heart Circ Physiol 301(1) (2011) H253-60.
[22] K. Bersell, S. Choudhury, M. Mollova, B. D. Polizzotti, B. Ganapathy, S. Walsh, B. Wadugu, S. Arab, B. Kuhn, Moderate and high amounts of tamoxifen in alphaMHC-MerCreMer mice induce a DNA damage response, leading to heart failure and death, Dis Model Mech 6(6) (2013) 1459-69.
[23] J. Lexow, T. Poggioli, P. Sarathchandra, M. P. Santini, N. Rosenthal, Cardiac fibrosis in mice expressing an inducible myocardial-specific Cre driver, Dis Model Mech 6(6) (2013) 1470-6.
[24] K. Muckova, J. S. Duffield, K. D. Held, J. V. Bonventre, A. M. Sheridan, cPLA2-interacting protein, PLIP, causes apoptosis and decreases G₁ phase in mesangial cells, Am J Physiol Renal Physiol 290(1) (2006) F70-9.
[25] Y. Tang, J. Luo, W. Zhang, W. Gu, Tip60-dependent acetylation of p53 modulates the decision between cell-cycle arrest and apoptosis, Mol Cell 24(6) (2006) 827-39.
[26] Y. Sun, X. Jiang, S. Chen, N. Fernandes, B. D. Price, A role for the Tip60 histone acetyltransferase in the acetylation and activation of ATM, Proc Natl Acad Sci USA 102(37) (2005) 13182-7.
[27] J. W. Lough, Transient expression of TIP60 protein during early chick heart development, Dev Dyn 223(3) (2002) 419-25.
[28] B. Li, Y. Hu, X. Li, G. Jin, X. Chen, G. Chen, Y. Chen, S. Huang, W. Liao, Y. Liao, Z. Teng, J. Bin, Sirt1 Antisense Long Noncoding RNA Promotes Cardiomyocyte Proliferation by Enhancing the Stability of Sirt1, J Am Heart Assoc 7(21) (2018) e009700.
[29] L. Peng, H. Ling, Z. Yuan, B. Fang, G. Bloom, K. Fukasawa, J. Koomen, J. Chen, W. S. Lane, E. Seto, SIRT1 negatively regulates the activities, functions, and protein levels of hMOF and TIP60, Mol Cell Biol 32(14) (2012) 2823-36.
[30] B. Li, M. Li, X. Li, H. Li, Y. Lai, S. Huang, X. He, X. Si, H. Zheng, W. Liao, Y. Liao, J. Bin, Sirt1-inducible deacetylation of p21 promotes cardiomyocyte proliferation, Aging (Albany N. Y.) 11(24) (2019) 12546-12567.
[31] T. M. A. Mohamed, Y. S. Ang, E. Radzinsky, P. Zhou, Y. Huang, A. Elfenbein, A. Foley, S. Magnitsky, D. Srivastava, Regulation of Cell Cycle to Stimulate Adult Cardiomyocyte Proliferation and Cardiac Regeneration, Cell 173(1) (2018) 104-116 e12.
[32] J. Zhou, F. Ahmad, S. Parikh, N. E. Hoffman, S. Raj an, V. K. Verma, J. Song, A. Yuan, S. Shanmughapriya, Y. Guo, E. Gao, W. Koch, J. R. Woodgett, M. Madesh, R. Kishore, H. Lal, T. Force, Loss of Adult Cardiac Myocyte GSK-3 Leads to Mitotic Catastrophe Resulting in Fatal Dilated Cardiomyopathy, Circ Res 118(8) (2016) 1208-22.
[33] C. Lemercier, G. Legube, C. Caron, M. Louwagie, J. Garin, D. Trouche, S. Khochbin, Tip60 acetyltransferase activity is controlled by phosphorylation, J Biol Chem 278(7) (2003) 4713-8.
[34] J. H. van Berlo, J. D. Molkentin, An emerging consensus on cardiac regeneration, Nat Med 20(12) (2014) 1386-93.
[35] P. Sdek, P. Zhao, Y. Wang, C. J. Huang, C. Y. Ko, P. C. Butler, J. N. Weiss, W. R. Maclellan, Rb and p130 control cell cycle gene silencing to maintain the postmitotic phenotype in cardiac myocytes, J Cell Biol 194(3) (2011) 407-23.
[36] P. Alam, B. Haile, M. Arif, R. Pandey, M. Rokvic, M. Nieman, B. D. Maliken, A. Paul, Y. G. Wang, S. Sadayappan, R. P. H. Ahmed, O. Kanisicak, Inhibition of Senescence-Associated Genes Rb1 and Meis2 in Adult Cardiomyocytes Results in Cell Cycle Reentry and Cardiac Repair Post-Myocardial Infarction, J Am Heart Assoc 8(15) (2019) e012089.
[37] A. I. Mahmoud, F. Kocabas, S. A. Muralidhar, W. Kimura, A. S. Koura, S. Thet, E. R. Porrello, H. A. Sadek, Meis1 regulates postnatal cardiomyocyte cell cycle arrest, Nature 497(7448) (2013) 249-253.
[38] T. Heallen, Y. Morikawa, J. Leach, G. Tao, J. T. Willerson, R. L. Johnson, J. F. Martin, Hippo signaling impedes adult heart regeneration, Development 140(23) (2013) 4683-90.
[39] R. Kerkela, L. Kockeritz, K. Macaulay, J. Zhou, B. W. Doble, C. Beahm, S. Greytak, K. Woulfe, C. M. Trivedi, J. R. Woodgett, J. A. Epstein, T. Force, G. S. Huggins, Deletion of GSK-3beta in mice leads to hypertrophic cardiomyopathy secondary to cardiomyoblast hyperproliferation, J Clin Invest 118(11) (2008) 3609-18.
[40] A. P. Singh, P. Umbarkar, Y. Guo, T. Force, M. Gupte, H. Lal, Inhibition of GSK-3 to induce cardiomyocyte proliferation: a recipe for in situ cardiac regeneration, Cardiovasc Res 115(1) (2019) 20-30.
[41] C. Charvet, M. Wissler, P. Brauns-Schubert, S. J. Wang, Y. Tang, F. C. Sigloch, H. Mellert, M. Brandenburg, S. E. Lindner, B. Breit, D. R. Green, S. B. McMahon, C. Borner, W. Gu, U. Maurer, Phosphorylation of Tip60 by GSK-3 determines the induction of PUMA and apoptosis by p53, Mol Cell 42(5) (2011) 584-96.
[42] S. Y. Lin, T. Y. Li, Q. Liu, C. Zhang, X. Li, Y. Chen, S. M. Zhang, G. Lian, K. Ruan, Z. Wang, C. S. Zhang, K. Y. Chien, J. Wu, Q. Li, J. Han, S. C. Lin, GSK3-TIP60-ULK1 signaling pathway links growth factor deprivation to autophagy, Science 336(6080) (2012) 477-81.
[43] T. Y. Li, L. Song, Y. Sun, J. Li, C. Yi, S. M. Lam, D. Xu, L. Zhou, X. Li, Y. Yang, C. S. Zhang, C. Xie, X. Huang, G. Shui, S. Y. Lin, K. Reue, S. C. Lin, Tip60-mediated lipin 1 acetylation and ER translocation determine triacylglycerol synthesis rate, Nature communications 9(1) (2018) 1916.
[44] X. Cheng, X. Ma, Q. Zhu, D. Song, X. Ding, L. Li, X. Jiang, X. Wang, R. Tian, H. Su, Z. Shen, S. Chen, T. Liu, W. Gong, W. Liu, Q. Sun, Pacer Is a Mediator of mTORC1 and GSK3-TIP60 Signaling in Regulation of Autophagosome Maturation and Lipid Metabolism, Mol Cell 73(4) (2019) 788-802 e7.
[45] T. Nie, S. Yang, H. Ma, L. Zhang, F. Lu, K. Tao, R. Wang, R. Yang, L. Huang, Z. Mao, Q. Yang, Regulation of ER stress-induced autophagy by GSK3beta-TIP60-ULK1 pathway, Cell death & disease 7(12) (2016) e2563.

### Example 2: Genetic Depletion of Tip60 Confers Regeneration of Infarcted Myocardium in Adult Mice

As shown in Example 1 and in prior published work by the inventors (Fisher et al PLoS One. 2012; 7:e31569. doi: 10.1371/journal.pone.0031569, PMID: 22348108; Fisher et al PLoS One. 2016; 11(10):e0164855. doi: 10.1371/journal.pone.0164855, PMID: 27768769), the early effects of Tip60 depletion to delay CM senescence raised the possibility that temporal, conditional depletion of Tip60 might be exploited in the setting of cardiac injury in adult animals reversing cellular senescence and conferring protection from apoptosis. Moreover, consideration of our findings vis-à-vis reports that activation of Atm—which requires prior acetylation by Tip60⁷⁻⁹—induces CM proliferative senescence in the neonatal²⁹ and the adult heart,³⁰ compelled testing of the hypothesis described in this example that Tip60 functions in adult CMs to maintain their non-proliferative status, preventing regeneration after cardiac injury.

In this Example, we describe experiments designed to test this possibility. Using mice containing foxed Kat5 alleles, combined with a conditional tamoxifen-activated Myh6-merCremer recombinase transgene, we report that Tip60 depletion subsequent to myocardial infarction (MI) maintained cardiac function by 10 days post-MI, a condition that was completely sustained until 28 days post-MI, when hearts were subjected to histologic determinations. Said histological analyses revealed that, at 28 days post-MI, Tip60-depleted hearts contained significantly less scar tissue, concomitant with increased numbers of CMs exhibiting cell cycle activation markers (Ki67, 5′-bromodeoxyuridine [BrdU], phospho histone H3 [pH3]) in both the remote and border zones, as well as a remarkable incidence of smooth muscle α-actin (SMA)-positive CMs in the border zone indicative of CM de-differentiation. In addition to increased cell cycle activity, the remote zone of Tip60-depleted hearts displayed decreased numbers of terminal deoxynucleotidyl transferase dUTP nick end labeling (TUNEL)-positive as well as cleaved caspase-3-positive cells, indicating protection from apoptosis. These findings demonstrate that depletion of Tip60 from adult CMs after MI preserves cardiac function, a phenomenon possibly mediated by the generation of new CMs combined with the preservation of existing cells. These findings advance our understanding of the molecular mechanisms that maintain proliferative senescence of CMs in the adult myocardium while suggesting a novel therapeutic target for restoring and maintaining cardiac muscle after MI.

### Materials and Methods:

Note: A more detailed description of the methods used in this study can be found in the Detailed Methods section that follows.

Animal care & experimentation: This investigation adhered to the National Institutes of Health (NIH) Guide for the Care and Use of Laboratory Animals (NIH Pub. Nos. 85-23, Revised 1996). All protocols described in the authors' Animal Use Application (AUA #000225), which were approved by the Medical College of Wisconsin's Institutional Animal Care and Use Committee (IACUC), were adhered to in this study. The IACUC has Animal Welfare Assurance status from the Office of Laboratory Animal Welfare (A3102-01).

Preparation of mice containing floxed Kat5 alleles, wherein Cre-recombinase removes exons 3-11 comprising two-thirds of the Tip60 coding sequence including the chromo and acetyltransferase domains, was recently described.²⁸ For these experiments, foxed mice were mated with a line (Jackson Laboratory #005650) containing an α-myosin heavy chain (Myh6)-driven merCremer-recombinase transgene, the product of which, upon administration of tamoxifen, enters the nucleus to recombine floxed alleles.³¹ All mice were on a mixed B6/sv129 genetic background. Experimental groups contained equivalent numbers of male and female littermates.

For the MI experiments, beginning on the third day after inducing MI by left main coronary artery ligation, mice received daily intraperitoneal injections of tamoxifen (40 mg/kg [Sigma #T5648] suspended with 5% ethanol in sunflower oil), for three consecutive days. Echocardiography was performed on a subset of mice at intervals following MI. On the day before harvest (28 days post-MI), mice were injected (1 mg) intraperitoneally with BrdU. On the day of harvest, mice were euthanized with CO₂ and hearts were perfused with ˜5 mL cardioplegic solution (25 mM KCl/5% dextrose in PBS) followed by 4% paraformaldehyde (PFA). After overnight fixation in PFA, hearts were transferred to 70% EtOH, followed by embedding in paraffin.

For qPCR and western blotting determinations, non-infarcted adult Kat5 floxed mice were given the three-day regimen of tamoxifen (40 mg/kg/day×3 days) and, 3-9 days after the 1^{st} dose of tamoxifen, hearts were harvested and apportioned for RNA and protein isolation by respectively placing samples in TRIzol (Thermo-Fisher #15596026) and RIPA buffer (Thermo-Fisher #89901) containing Halt anti-protease/anti-phosphatase cocktail (Thermo-Fisher #78440). Samples were minced and homogenized with a teflon pestle and stored at −80° C. until further processing.

Quantitative assessment of myocardial scarring: Paraffinized hearts were transversely sectioned, in entirety, from apex to base, after which eight 4 μm thick sections from equidistant (˜0.8 mm) intervals were placed on microscope slides. The slides were stained with Masson trichrome to quantitatively assess scar size.³² Briefly, trichrome-stained sections were examined with a Nikon SMZ800 microscope and photographed at 10× magnification using a SPOT Insight camera (Nikon Instruments). MIQuant software was used to quantitate infarct size in sections between the apex and the ligation suture site, as previously described.³² Results were expressed as the average percentage of area and midline length around the left ventricle.

Quantitative assessment of cell cycle activation: Heart tissue was processed for histology, followed by immunostaining cell cycle activation markers BrdU, Ki67, and pH3, and counter-staining cardiac troponin T (cTnT) to verify CM identity, as described in detail in the Detailed Methods section. Fluorescent signals were photographed in six randomly selected fields of the left ventricle at the magnification indicated in each figure legend using a Nikon Eclipse 50i microscope equipped with a Nikon DSU3 digital camera. To quantify the extent of CM cell cycle activation, myonuclei (≥1.5 μm diameter) exhibiting signal comprising >50% of the nuclear area and confirmed to be surrounded by cTnT-positive cytoplasm were enumerated in each field by a blinded observer; cells in which nuclei did not conform to these standards were identified as non-cardiomyocytes (non-CMs). Approximately 1,000 CMs were evaluated in each section (heart). Results are presented as the average number of events per field.

Statistics: All determinations were performed in blinded fashion and are reported as means±SEM. Echocardiography data were analyzed by a two-way repeated measures ANOVA (time and genotype) to determine whether there was a main effect of time, genotype, or a time-genotype interaction. If global tests showed an effect, post hoc contrasts between baseline and subsequent timepoints within experimental groups were compared by a Dunnett's multiple comparison t test; differences between genotypes at each timepoint were compared by a Student's t test with the Bonferroni correction. All other data were compared by an unpaired, two-tailed Student's t test.

### Detailed Methods:

Myocardial infarction and echocardiography: To induce myocardial infarction (MI), mice were respirated (model 845, Harvard Apparatus) via an endotracheal tube with room air supplemented with 100% oxygen to maintain blood gases within normal physiological limits. The electrocardiogram (ECG; limb lead II configuration) was continuously recorded (Powerlab) using needle electrodes and rectal temperature was maintained at 37° C. throughout the experiments using a servo-controlled heating pad. Once the mice were anesthetized and prepared for surgery, thoracotomy was performed to the left of the sternum to expose the heart, followed by opening of the pericardium and placement of an 8.0 nylon suture beneath the left main coronary artery at a level below the tip of the left atrium to target the lower half of the ventricle, with the aid of a microscope. Ischemia was induced by carefully tying the suture with a double knot, after which coronary occlusion was verified by visual observation of blanching of the myocardium distal to the ligature and by ST segment elevation on the ECG. After ligation, the chest wall was closed with polypropylene suture and recovery was monitored until mice became fully ambulatory. Immediately prior to initiating the surgical procedure to produce MI, mice were injected subcutaneously with sustained release meloxicam (4 mg/kg) to limit post-operative pain.

At scheduled intervals, echocardiographic assessment (VisualSonics Vevo 770 or 3100 high-frequency ultrasound imaging systems) was performed on mice lightly anesthetized with isoflurane delivered via a nose cone in the parasternal long-axis, short-axis, and apical 4-chamber views using a transducer (RMV 707 or MX550D) operating at 30-40 mHz. The parasternal views in M-mode were used to measure left ventricular anteroposterior internal diameter (LVID), anterior wall thickness (LVAW), and posterior wall thickness (LVPW) at end-diastole (d) and end-systole (s) at the mid-ventricular level. Left ventricular systolic function was assessed by fractional shortening: FS (%)=([LVIDd−LVIDs]/LVIDd)*100. In addition, global left ventricular function was assessed by calculating the myocardial performance index: MPI=(isovolumic contraction time+isovolumic relaxation time)/ejection time. Time intervals were obtained from pulsed Doppler waveforms of mitral valve inflow and aortic valve outflow from apical 4-chamber views.

Genotyping was performed by PCR in 20 μl reactions that included 2× GoTaq Green Master Mix (Promega #M7123), 1.1 mM MgCl₂, 0.5 μM each primer, 0.5 μM internal control primers, and 4.0 μl template. Templates consisted of supernatants of ear tissue samples that had been boiled for 10 minutes in 0.3 mL 10 mM NaOH/1 mM EDTA. Sequences of primer pairs used for PCR are listed in Table 4. PCR products were amplified in an AB Applied Biosystems GeneAmp PCR System 9700 using the following programs: for LoxP, one 5 minute cycle at 95° C., thirty-five cycles at 94° C. 30 sec/61° C. 45 sec/72° C. 45 sec, followed by one 10 minute cycle at 72° C.; for Myh6-merCremer, one 5 minute cycle at 95° C., thirty-five cycles at 94° C. 30 sec/54° C. 45 sec/72° C. 45 sec, followed by one 10 minute cycle at 72° C. Amplicons were separated at 100-110 V for one hour in 1% agarose with ethidium bromide and imaged.

Synthetic-Primer

TABLE 4
Primers & Probes
for PCR Genotyping

| Allele | Sequence (5′-3′) & Working Conc. | Amplicon (bp) | Annealing ° C. |
|---|---|---|---|
| LoxP | FWD 0.5 μM | 687 LoxP | 61 |
| in intron 2 | GGAGGGAGTCAACGATCGCA | 586 WT | |
| | (SEQ ID NO: 7) | | |
| | REV 0.5 μM | | |
| | AATGGGGGACCTACTCACCA | | |
| | (SEQ ID NO: 8) | | |

Cycling Details: 94° C. 5 min, then 35 cycles of 94° C. 30 sec/61° C. 45 sec/
72° C. 45 sec, then 72° C. 10 min

| | | | |
|---|---|---|---|
| LoxP | FWD 0.5 μM | 655 LoxP | 61 |
| in intron 11 | GCACTCATCCAGGCTGTCC | 554 WT | |
| | (SEQ ID NO: 9) | | |
| | REV 0.5 μM | | |
| | TCGGTTCTCAGAGACTAGC | | |
| | (SEQ ID NO: 4) | | |

Cycling Details: 94° C. 5 min, then 35 cycles of 94° C. 30 sec/61° C. 45 sec/
72° C. 45 sec, then 72° C. 10 min

| | | | |
|---|---|---|---|
| Myh6-Cre | FWD 0.5 μM | ~440 | 54 |
| transgene | ATACCGGAGATCATGCAAGC | | |
| | (SEQ ID NO: 10) | | |
| | REV 0.5 μM | | |
| | AGGTGGACCTGATCATGGAG | | |
| | (SEQ ID NO: 11) | | |

Cycling Details: 94° C. 5 min, then 35 cycles of 94° C. 30 sec/54° C. 45 sec/
72° C. 45 sec, then 72° C. 10 min
for Taqman qRT-PCR

| Gene Target | Taqman Probe Kit (Thermo-Fisher #) |
|---|---|
| Gapdh | Mm99999915_g1 |
| Kat5 (Tip60) | Mm01231512_m1 |

Quantitative RT-PCR (qPCR): Heart tissue, previously disrupted by homogenization with a motorized (Kimble 749540-0000) Teflon pestle and stored at −80° C. in TRIzol reagent, was thawed. RNA was immediately purified using PureLink RNA Mini-Kits (ThermoFisher #12183018A), including a genomic DNA removal step (PureLink DNase kit for on-column protocol, Thermo-Fisher #12185-010), according to the manufacturer's instructions. RNA yield & quality were determined via 260/280 ratio using Eppendorf Biophotometer Plus Instrument.

cDNA was synthesized as follows. After diluting an RNA sample from each heart so that precisely 1.0 μg was suspended in 14 μl nuclease-free distilled water (NFDW), 4.0 μl 5× VILO reaction mixture (ThermoFisher #11754050) were added. To start the reverse-transcription reaction, 2.0 μl 10× SuperScript Enzyme Mix (ThermoFisher #11754050) were added, followed by transfer to an Applied Biosystems Veriti 96-well Thermocycler programmed as follows: 10 minutes at 25° C.→60 minutes at 42° C.→5 minutes at 85° C. cDNA templates were diluted with NFDW to a concentration of 6.25 ng/μ1 and stored at −20° C.

qPCR was carried-out by subjecting each biological replicate (i.e. sample from each individual heart) to triplicate determinations. Each reaction was performed in a total volume of 20 μl in 96-well arrays, each well containing 1× Taqman Fast-Advanced Master Mix (ThermoFisher #4444557), 1× Taqman Probe Kit (Table 4), and 25 ng cDNA as template. The arrayed samples were amplified in a Bio-Rad CFX96 Real Time System (C1000 Touch) programmed as follows: 2 minutes at 50° C.→20 seconds at 95° C.→3 seconds at 95° C.→30 seconds at 60° C.; the last two steps were repeated 39 times. Results were processed using Bio-Rad CFX Manager 3.1 software.

Western blotting: Upon harvesting in ice-cold RIPA Lysis and Extraction buffer (ThermoFisher #89900) fortified with Halt Protease and Inhibitor Cocktail (ThermoFisher #78440), samples were finely minced, homogenized, and stored at −80° C. Prior to electrophoresis, tissues were thawed at 0° C. and homogenized with a motorized Teflon pestle, followed by determination of total protein concentration using a Standard Bradford Assay (Bio-Rad #500-0006) and dilution in Laemmli Sample Buffer (Bio-Rad #161-0747) to a concentration of 2.5 mg/mL. For electrophoresis, 20 μg of each sample were loaded into each lane of a pre-cast Bio-Rad 4-20% acrylamide gel, and separated proteins were transferred (60 min at 100 V) onto 0.45 μm nitrocellulose membrane (Bio-Rad #162-0145). The blots were blocked with 5% non-fat dry milk/10 mM Tris-HCl (pH 7.6)/150 mM NaCl/Tween-20 (5% NFDM/TBST) or 5% BSA in TBST. Primary and secondary antibodies and dilutions are listed in Table 2. Blots were reacted with primary antibody in 5% NFDM/TBST or 5% BSA blocking buffer overnight at 4° C. Secondary antibodies were diluted in 5% NFDM/TBST and applied for 60 minutes at RT. Reacted blots were covered with horseradish peroxidase substrate (ThermoFisher #34580) for 5 min at room temperature, followed by chemiluminescence imaging and densitometry using Bio-Rad ChemiDoc and ImageJ software, respectively.

Immunostaining & cell counting: As described in the narrative, hearts were perfused with ˜5 mL cardioplegic solution (25 mM KCl/5% dextrose in PBS) followed by 4% paraformaldehyde (PFA). After overnight fixation in PFA, hearts were transferred to 70% EtOH, followed by embedding in paraffin, sectioning at 4 μm thickness, and placement of sections on microscope slides. For staining, sections were de-waxed and subjected to antigen retrieval (100° C. in 10 mM trisodium citrate [pH6.0]/0.05% Tween-20 for 20 minutes), followed by 30 min cooling at room temperature, and blocking with 2% goat serum/0.1% Triton X-100 in PBS. Primary antibodies were diluted in blocking buffer and applied overnight at 4° C.; secondary antibodies were applied for one hour in the dark. Combinations of primary and secondary antibodies employed for each antigen, plus dilutions, are shown in Table 5.

TABLE 5
Antibodies for Immunofluorescent Staining & Western Blotting

| | | | | |
|---|---|---|---|---|
| Antigen | Manufacturer | Catalog # | Made in | Dilution |

Immunostaining

| | | | | |
|---|---|---|---|---|
| 1° 5′-bromodeoxyuridine | Abcam | ab6326 | rat | 1:200 |
| (BrdU) | | | | |
| 2° goat anti-rat 594 | Invitrogen | A-11007 | goat | 1:500 |
| 1° phosphohistone H3 | EMD | 06-570 | rabbit | 1:400 |
| (pH 3) | Millipore | | | |
| 2° goat anti-rabbit 594 | Invitrogen | A-11037 | goat | 1:500 |
| 1° Ki67 | Invitrogen | 14-5698-82 | rat | 1:250 |
| 2° goat anti-rat 594 | Invitrogen | A-11007 | goat | 1:500 |
| 1° cardiac-Troponin | Abcam | Ab8295 | mouse | 1:200 |
| (cTnT) | | | | |
| 2° goat anti-mouse 488 | Invitrogen | A-11029 | goat | 1:500 |
| 1° smooth muscle actin | DAKO | M0851 | mouse | 1:100 |
| (SMA) | | | | |
| 2° goat anti-mouse 488 | Invitrogen | A-11029 | goat | 1:500 |
| 1° cleaved caspase-3 | Cell | 9661 | rabbit | 1:50 |
| (Asp175) | Signaling | | | |
| 2° goat anti-rabbit 594 | Invitrogen | A-11007 | goat | 1:500 |

Wheat Germ Agglutinin Staining

| | | | |
|---|---|---|---|
| Wheat Germ | Thermo | W11261 | 50 μg/ml |
| Agglutinin-488 | Fisher | | |

Western Blotting

| | | | | |
|---|---|---|---|---|
| 1° Tip60 | Bethyl | custom | rabbit | 1:1000 |
| 2° goat anti-rabbit IgG, | Thermo | 32460 | goat | 1:5000 |
| HRP | Fisher | | | |

Slides were stained to detect markers for nuclear cell cycle activation (Ki67, 5′-bromodeoxyuridine [BrdU], phospho histone H3 [pH3]), a marker of cardiomyocyte identity (cardiac Troponin T [cTnT]), and smooth muscle α-actin (SMA). Microscopy was performed at 200× magnification using a Nikon Eclipse 50i microscope equipped with a Nikon DSU3 digital camera. For each heart, six microscopic fields in sections representing (i) the remote zone, specifically within an area of myocardium mm distal to the boundary of the infarct, and (ii) the border zone, specifically the area immediately adjacent to the infarct zone, were randomly selected for counting, which was manually performed by blinded observers. Double-positive cells were counted and presented as the average number of events per microscopic field.

Terminal deoxynucleotidyl transferase dUTP nick end labeling (TUNEL): Apoptosis was assessed using the DeadEnd Fluorometric TUNEL System (Promega #G3250) per the manufacturer's instructions. The total number of TUNEL-positive cells within sections representing the border and remote zones were manually counted at 400× magnification. TUNEL signal was counted only if confined to a DAPI-positive nucleus. Nuclei were scored as TUNEL-positive only if at least 50% of the nucleus contained fluorescent signal.

Wheat germ agglutinin (WGA) staining was performed using Thermo-Fisher #W11261 Alexa Fluor 488 conjugate. Sections mounted on microscope slides were stained with 50 μg/ml WGA in PBS for 10 minutes at room temperature, followed by thorough washing. Images of CMs at 400× magnification in transverse orientation were photo-micrographed as described above and processed to determine average pixel numbers/CM, as indicative of CM size, using ImageJ software. Briefly, the green (488) channel displaying CMs outlined in cross-section was isolated, followed by thresholding to fill-in spaces occupied by CM cytoplasm, then adjusting the settings to acquire particle sizes in the 100-infinity range having a circularity of 0.2-100. After results (which were set to “include holes”) were obtained, particles representing incorrectly oriented CMs or blood vessels were removed.

### Results:

### Experimental Scheme and Impact of Conditional Tip60 Depletion on Non-Injured Mice

Our objective was to assess whether effects of MI could be minimized by subsequently depleting Tip60. Experimentally, we assessed the effect of administering tamoxifen on days 3-5 post-MI to control mice containing foxed Kat5 alleles (Kat5^{f/f}), in comparison with identical mice containing a Myh6-merCremer-recombinase transgene³¹ (Kat5^{flox/flox;Myh6-merCremer} denoted Kat5^{Δ/Δ}). This was followed by experiments to control for off-target effects of the Cre transgene per se, by comparing wild-type (i.e., Kat5^{+/+}) with Kat5^{+/+;Myh6-merCremer} genotypes. The experimental timeline consisted of a 28-day post-MI follow-up period to permit regeneration and healing to become manifest.

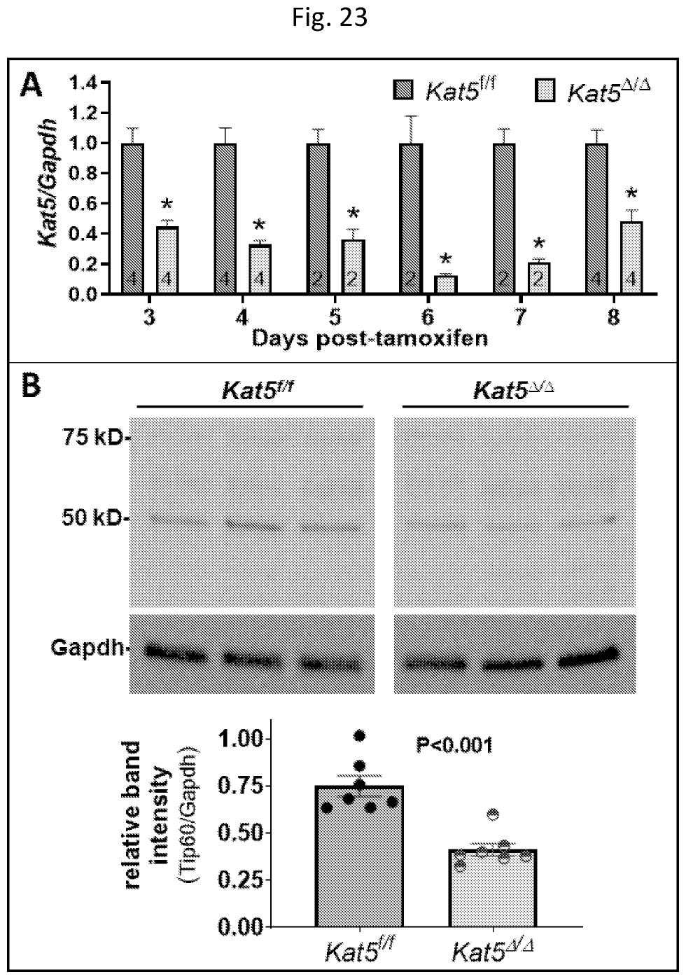

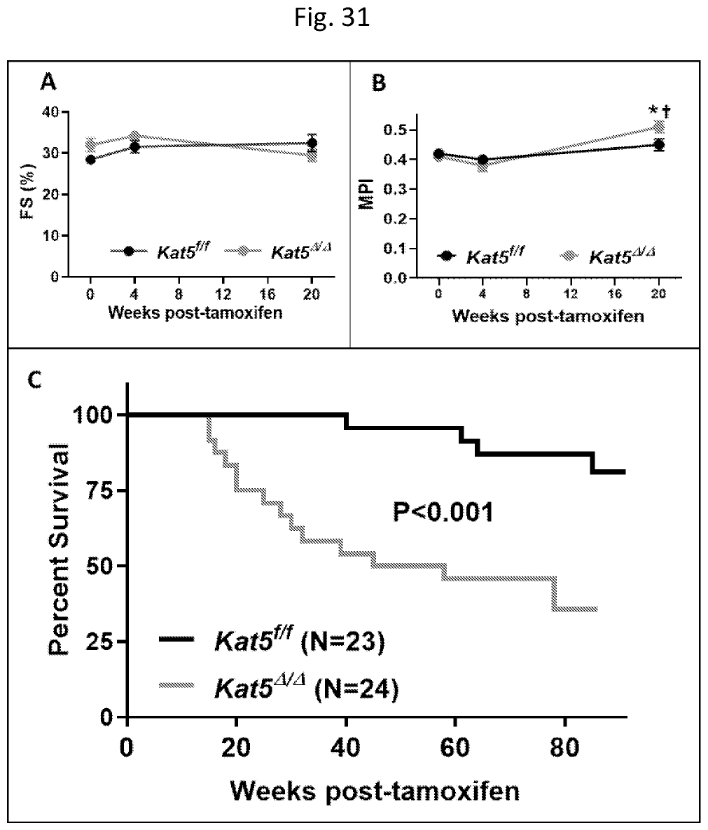

Prior to performing MI experiments, the effect of administering three consecutive daily doses of tamoxifen (40 mg/kg) to non-infarcted adult mice was assessed to determine the extent of conditional Tip60 depletion, and, because we had previously observed that Tip60 is a vital protein in CMs,²⁸ to assess whether its depletion compromised cardiac function and longevity. As shown in FIG. 23, levels of Kat5 transcripts (FIG. 23A) and Tip60 protein (FIG. 23B) were both depleted ≥50% in hearts of Kat5^{Δ/Δ} mice as early as 3 days after the first dose of tamoxifen. It is likely that depletion in CMs was even more extensive, since Tip60 was not depleted in non-CMs, which comprise a majority of the cell types in the adult mouse heart.³³ FIG. 31 shows echocardiographic and survival data from non-injured mice, revealing normal function of Tip60-depleted hearts at the experimental endpoint (4 weeks post-tamoxifen). Kat5^{Δ/Δ} mice did not begin to die until four months later, at 20 weeks post-tamoxifen (FIG. 31C) when cardiac dysfunction became apparent (FIG. 31A-B); all indices of cardiac function in non-injured mice are shown in Table 6. Hence, no untoward effects of Tip60 depletion were noted during the 28-day (4-week) timeline proposed for the MI experiments.

TABLE 6
Comparison of Echocardiography Assessments in Non-injured Tip60-depleted
Mice: Tip60-depleted (Kat5^{Δ/Δ}) versus Control (Kat5^{f/f}).

| | baseline | | 4 weeks-post-tamoxifen | | 20 weeks-post-tamoxifen | |
|---|---|---|---|---|---|---|
| | Kat5^{f/f} | Kat5^{Δ/Δ} | Kat5^{f/f} | Kat5^{Δ/Δ} | Kat5^{f/f} | Kat5^{Δ/Δ} |
| | N = 23 | N = 23 | N = 23 | N = 23 | N = 23 | N = 18 |
| LVAWd (mm) | 0.89 ± 0.02 | 0.83 ± 0.02 | 0.94 ± 0.02 | 0.92 ± 0.03† | 1.00 ± 0.03† | 0.93 ± 0.02† |
| LVAWs (mm) | 1.24 ± 0.03 | 1.22 ± 0.03 | 1.33 ± 0.03 | 1.30 ± 0.03 | 1.44 ± 0.05† | 1.33 ± 0.03 |
| LVIDd (mm) | 3.93 ± 0.06 | 3.74 ± 0.06 | 3.86 ± 0.08 | 3.60 ± 0.08 | 3.82 ± 0.11 | 3.80 ± 0.10 |
| LVIDs (mm) | 2.82 ± 0.07 | 2.55 ± 0.08 | 2.66 ± 0.10 | 2.37 ± 0.07 | 2.62 ± 0.14 | 2.69 ± 0.11 |
| LVPWd (mm) | 0.75 ± 0.03 | 0.74 ± 0.02 | 0.82 ± 0.03 | 0.78 ± 0.02 | 0.89 ± 0.03† | 0.87 ± 0.03† |
| LVPWs (mm) | 1.04 ± 0.03 | 1.10 ± 0.03 | 1.17 ± 0.04† | 1.14 ± 0.04 | 1.23 ± 0.05† | 1.17 ± 0.04 |
| HR (bpm) | 428 ± 7 | 458 ± 11 | 474 ± 9† | 469 ± 13 | 487 ± 16† | 444 ± 18 |
| FS | 28.5 ± 1.0 | 32.0 ± 1.5 | 31.6 ± 1.5 | 34.3 ± 1.2 | 32.5 ± 2.0 | 29.5 ± 1.4 |
| (%) | | | | | | |
| MPI | 0.42 ± 0.01 | 0.41 ± 0.01 | 0.40 ± 0.01 | 0.38 ± 0.02 | 0.45 ± 0.02 | 0.51 ± 0.02*† |
| LV mass (mg) | 94.6 ± 3.0 | 81.5 ± 2.9 | 101.1 ± 4.1 | 85.8 ± 2.8* | 112.0 ± 5.4† | 101.6 ± 3.8† |

*P < 0.05 vs. Kat5^{f/f} and
†P < 0.05 vs. baseline.

Tip60 Depletion Preserves Cardiac Function and Reduces Myocardial Scarring after MI

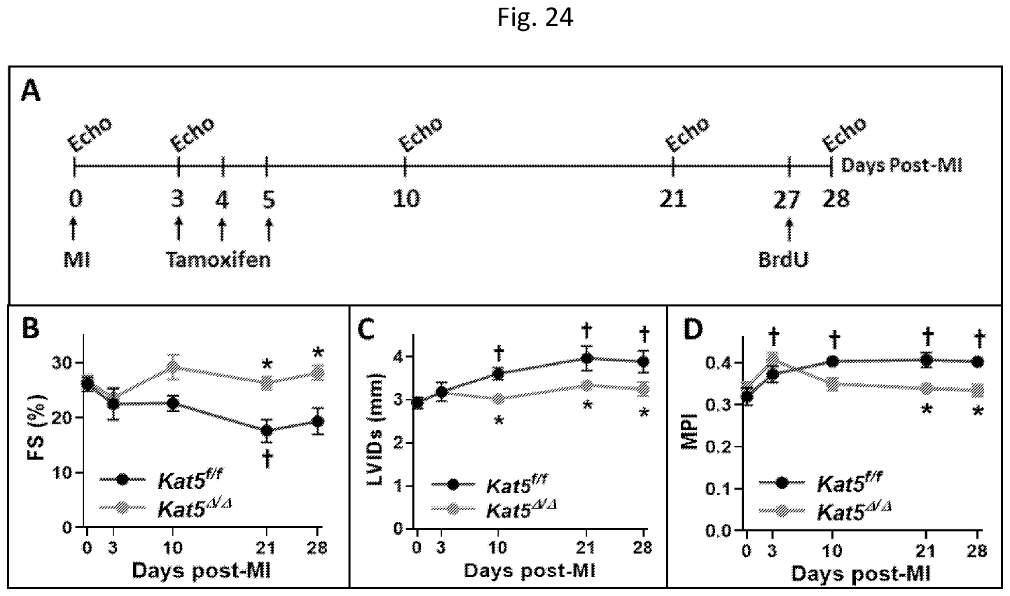

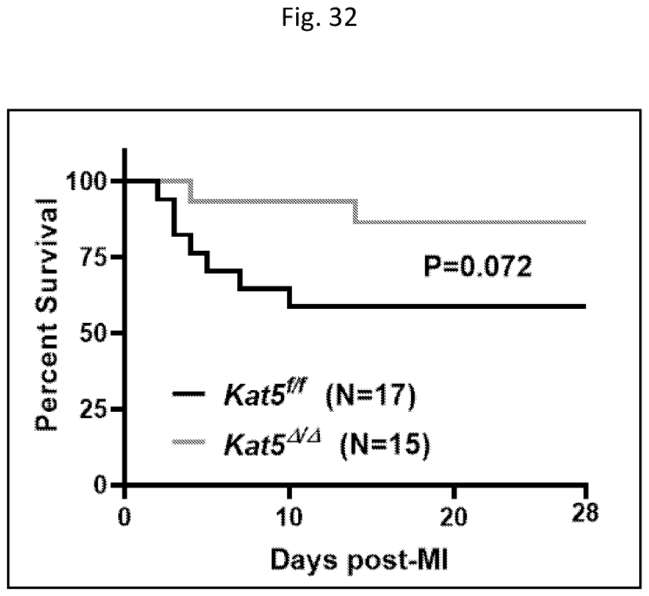

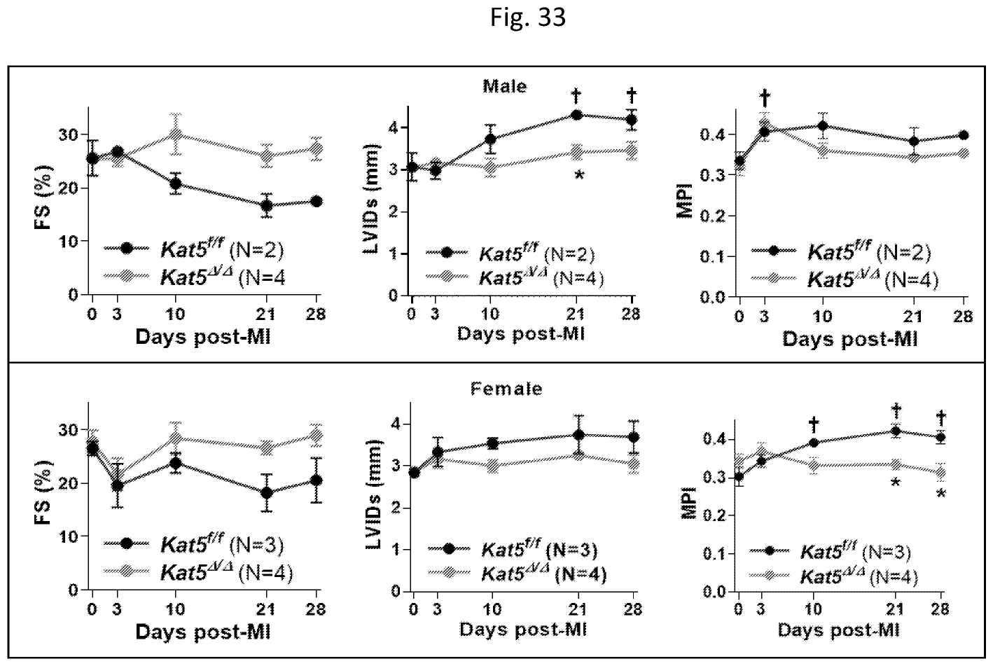

The impact of Tip60 depletion on the effects of MI was then addressed. The method of MI employed in this study was designed to generate relatively small, uniform infarctions by permanently ligating the left main coronary artery at a position below the tip of the left atrium to target the distal half of the left ventricle. As indicated by the experimental timeline shown in FIG. 24A, echocardiography was performed on infarcted Kat5^{f/f} and Kat5^{Δ/Δ} mice at intervals up to 28 days post-MI. Remarkably, one week after the first tamoxifen injection—at day 10 post-MI—all indices of left ventricular function assessed by echocardiography were normal in Kat5^{Δ/Δ} mice in comparison with Kat5^{f/f} controls (FIG. 24B-D). These indices included fractional shortening (FS; FIG. 24B), end-systolic diameter (LVIDs; FIG. 24C), and the myocardial performance index (MPI, FIG. 24D), which is a global measure of ventricular performance based on the ratio of the sum of isovolumic contraction and relaxation times to the ejection time, determined from Doppler recordings of mitral valve inflow and aortic valve outflow waveforms. All measures were maintained at pre-MI baseline (day 0) values between the 10- and 28-day post-MI time-points (FIG. 24B-D; summary of all echocardiographic data is shown in Table 7). Preservation of function in Tip60-depleted hearts is consistent with survival data (FIG. 32), which indicated a trend toward improved survival of Kat5^{Δ/Δ} mice. Functional improvement with Tip60 depletion was evident in both male and female mice (FIG. 33)

TABLE 7
Comparison of Echocardiography Assessments of Infarcted Tip60-depleted Mice:
Tip60-depleted Kat5^{Δ/Δ}) versus Control (Kat5^{f/f}).

| | Baseline | | 3 days-post-MI | | 10 days-post-MI | |
|---|---|---|---|---|---|---|
| | Kat5^{f/f} | Kat5^{Δ/Δ} | Kat5^{f/f} | Kat5^{Δ/Δ} | Kat5^{f/f} | Kat5^{Δ/Δ} |
| | N = 5 | N = 8 | N = 5 | N = 8 | N = 5 | N = 8 |
| LVAWd | 0.72 ± 0.01 | 0.76 ± 0.02 | 0.84 ± 0.04 | 0.86 ± 0.03† | 0.75 ± 0.04 | 0.85 ± 0.01 |
| (mm) | | | | | | |
| LVAWs | 1.00 ± 0.05 | 1.05 ± 0.02 | 1.06 ± 0.09 | 1.07 ± 0.06 | 0.87 ± 0.08 | 1.16 ± 0.04* |
| (mm) | | | | | | |
| LVIDd | 3.96 ± 0.12 | 4.03 ± 0.06 | 4.10 ± 0.15 | 4.15 ± 0.10 | 4.67 ± 0.14† | 4.27 ± 0.07 |
| (mm) | | | | | | |
| LVIDs | 2.93 ± 0.13 | 2.96 ± 0.06 | 3.19 ± 0.22 | 3.17 ± 0.11 | 3.61 ± 0.14† | 3.02 ± 0.12* |
| (mm) | | | | | | |
| LVPWd | 0.70 ± 0.04 | 0.75 ± 0.02 | 0.82 ± 0.021* | 0.77 ± 0.02 | 0.75 ± 0.03 | 0.76 ± 0.02 |
| (mm) | | | | | | |
| LVPWs | 0.93 ± 0.03 | 1.00 ± 0.04 | 0.99 ± 0.02 | 1.01 ± 0.03 | 0.99 ± 0.09 | 1.02 ± 0.03 |
| (mm) | | | | | | |
| HR | 377 ± 21 | 418 ± 14 | 469 ± 23† | 464 ± 13 | 435 ± 20 | 429 ± 14 |
| (bpm) | | | | | | |
| FS | 26.2 ± 1.3 | 26.6 ± 1.1 | 22.5 ± 2.9 | 23.7 ± 1.7 | 22.6 ± 1.4 | 29.2 ± 2.3 |
| (%) | | | | | | |
| MPI | 0.32 ± 0.02 | 0.34 ± 0.02 | 0.37 ± 0.02 | 0.41 ± 0.02† | 0.40 ± 0.01† | 0.35 ± 0.02 |
| LV mass | 79.3 ± 5.0 | 87.8 ± 3.7 | 102.4 ± 5.6 | 102.5 ± 6.6 | 111.8 ± 6.1† | 105.2 ± 3.8 |
| (mg) | | | | | | |
| | | | 21 days-post-MI | | 28 days-post-MI | |
| | | | Kat5^{f/f} | Kat5^{Δ/Δ} | Kat5^{f/f} | Kat5^{Δ/Δ} |
| | | | N = 5 | N = 8 | N = 5 | N = 8 |
| | | LVAWd | 0.78 ± 0.01 | 0.83 ± 0.02 | 0.73 ± 0.08 | 0.83 ± 0.01 |
| | | (mm) | | | | |
| | | LVAWs | 0.93 ± 0.04 | 1.11 ± 0.04 | 0.93 ± 0.12 | 1.14 ± 0.02* |
| | | (mm) | | | | |
| | | LVIDd | 4.79 ± 0.26† | †4.52 ± 0.08† | 4.80 ± 0.21† | 4.52 ± 0.15† |
| | | (mm) | | | | |
| | | LVIDs | 3.97 ± 0.29† | †3.34 ± 0.10* | 3.89 ± 0.25† | 3.25 ± 0.16* |
| | | (mm) | | | | |
| | | LVPWd | 0.76 ± 0.03 | 0.76 ± 0.02 | 0.74 ± 0.03 | 0.75 ± 0.02 |
| | | (mm) | | | | |
| | | LVPWs | 0.92 ± 0.04 | 0.99 ± 0.01 | 0.94 ± 0.06 | 1.00 ± 0.03 |
| | | (mm) | | | | |
| | | HR | 450 ± 23† | 414 ± 10 | 407 ± 22 | 408 ± 14 |
| | | (bpm) | | | | |
| | | FS | 17.6 ± 2.1† | 26.3 ± 1.2* | 19.4 ± 2.4 | 28.2 ± 1.4* |
| | | (%) | | | | |
| | | MPI | 0.41 ± 0.02† | †0.34 ± 0.01* | 0.40 ± 0.01† | 0.33 ± 0.01* |
| | | LV mass | 121.6 ± 9.9† | 114.7 ± 6.1†† | 114.2 ± 7.4† | 114.7 ± 7.0† |
| | | (mg) | | | | |

*P < 0.05 vs. Kat5^{f/f} and
†P < 0.05 vs. baseline (Day 0).

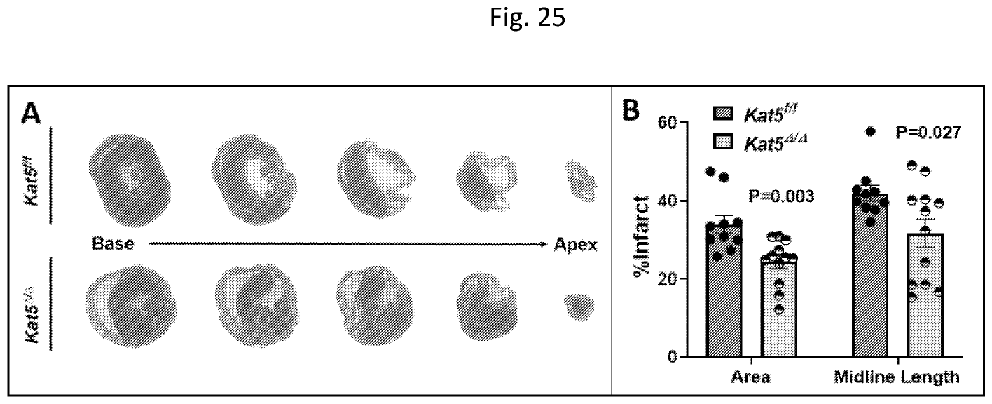

In agreement with improved function observed by echocardiography, we observed significantly decreased scarring in the myocardium at 28 days post MI (FIG. 25). For this assessment, hearts from 28 days post-MI mice were fixed and transversely sectioned from apex to base, followed by Masson trichrome staining (FIG. 25A). Total scar area and scar midline length were quantitatively assessed in blind as previously described,³² by digitizing areas occupied by blue staining between the apex and the site of ligation (FIG. 25B). This revealed that scarring, as evaluated by both parameters, was significantly diminished by 25-30% in the myocardium of Tip60-depleted Kat5^{Δ/Δ} mice.

Tip60 Depletion after MI is Accompanied by Cell Cycle Activation and SMA Expression in CMs, Concomitant with Reduced Apoptosis

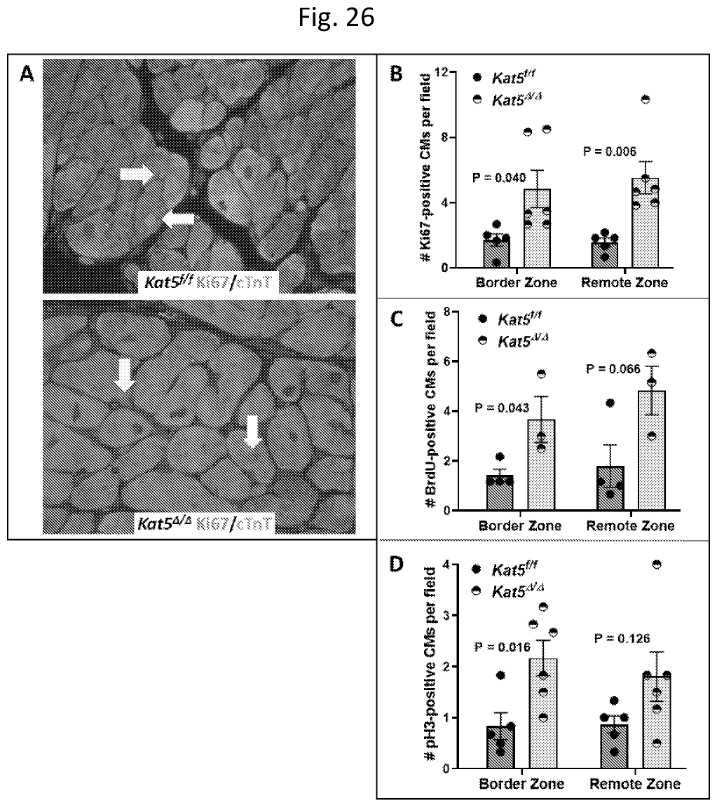

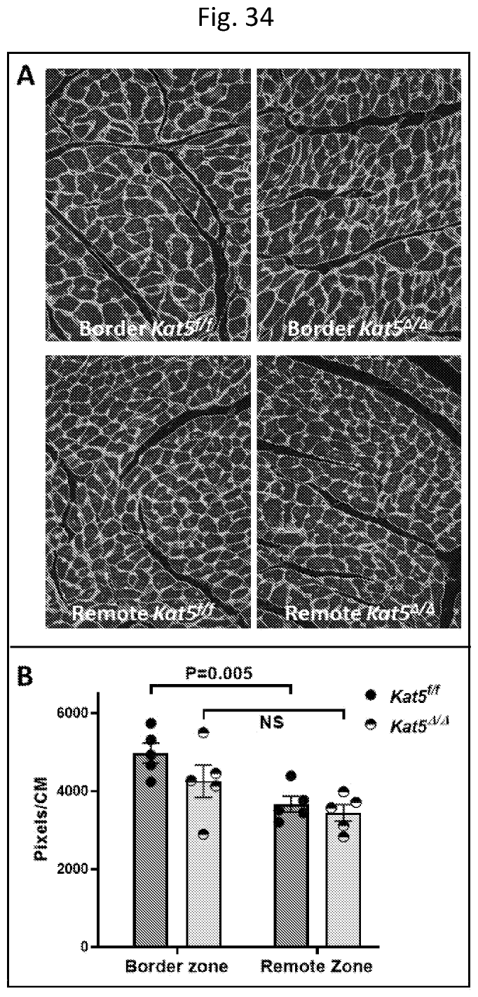

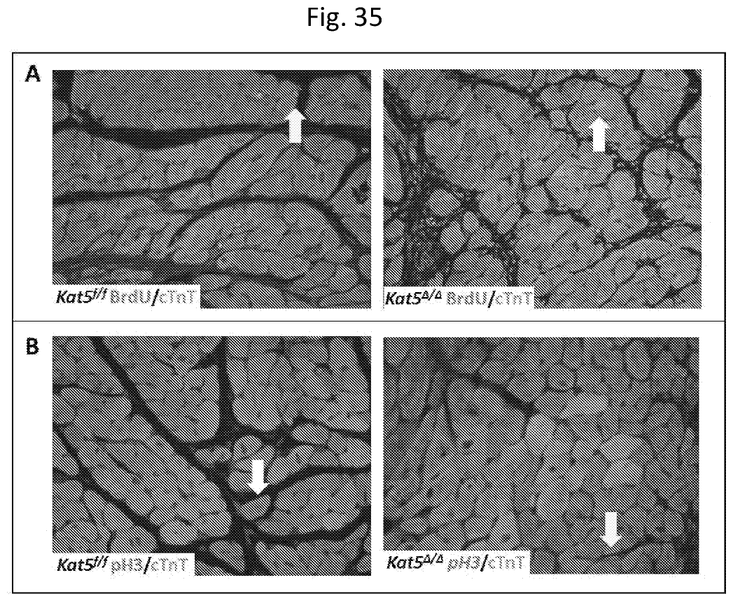

Observations of preserved cardiac function and muscle mass in infarcted Tip60-depleted hearts could be explained by CM proliferation, protection from ischemia-induced cell death, or hypertrophic growth of the remaining myocardium. These possibilities were examined as shown in FIGS. 26-28, and in FIG. 34. FIG. 26 demonstrates that Tip60 depletion was associated with >two-fold increases, most of which were statistically significant (P<0.05), in the number of CMs exhibiting increased cell cycle activation at 28 days post-MI. Increased numbers of cycling CMs were observed in both the border and remote zones of Tip60-depleted myocardium in Kat5^{Δ/Δ} hearts (FIG. 26B-D), although cycling CMs were not detected in the infarct zone. Cell cycle activation was documented using three indicators—Ki67 (FIG. 26A-B), BrdU (FIG. 26C & FIG. 35A), and pH3 (FIG. 26D & FIG. 35B)—markers that respectively detect all cell cycle phases, S-phase, and early M-phase.

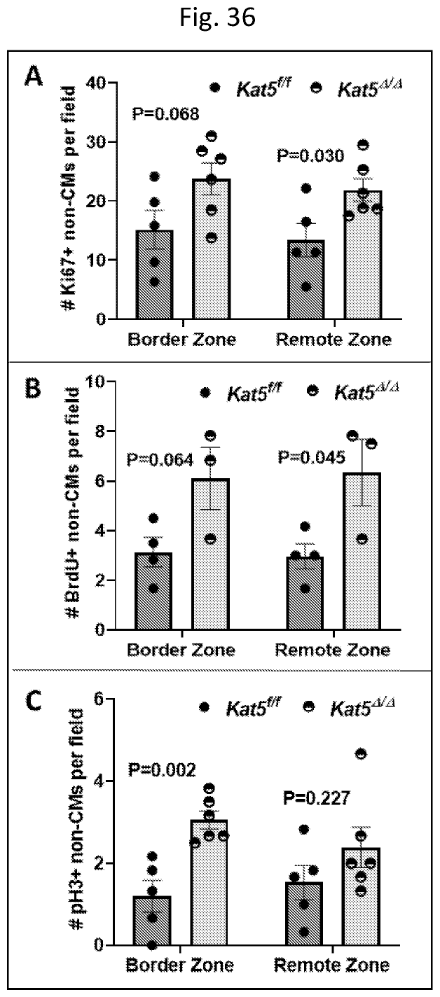

Unknown cell types that did not exhibit cTnT staining, the marker of CM identity employed in these determinations, also displayed increased cell cycle activation in Kat5^{Δ/Δ} hearts at 28 post-MI (non-CMs; FIG. 36). Because Tip60 was theoretically depleted only in CMs, this unexpected finding suggests involvement of a paracrine-mediated response.

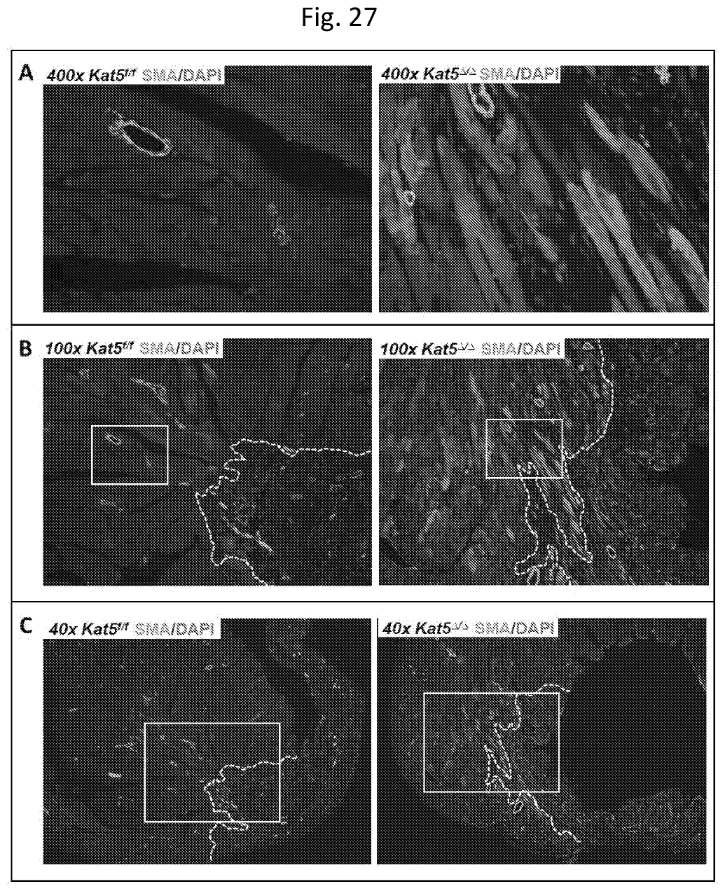

FIG. 27 displays the surprising finding that areas within the infarct border zone of 28 days post-MI Kat5^{Δ/Δ} hearts contained a high density of SMA-positive CMs, which was not observed in Kat5^{f/f} control hearts. This phenomenon suggests the presence of de-differentiated CMs, as documented in regenerating myocardium,³⁴ and/or early developing CMs, since SMA is transiently expressed in CMs during initial stages of embryonic heart development.^{35, 36}

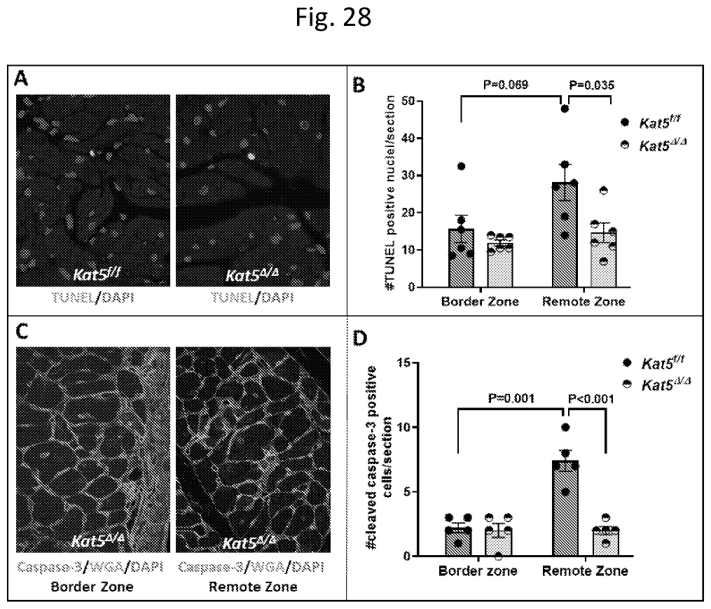

FIG. 28 shows that at 28 days post-MI, numbers of TUNEL-positive cells (FIG. 28 A,B), as well as cleaved caspase-3-positive cells (FIG. 28 C,D), both of which are indicative of apoptosis, were equivalent in the border zone of Kat5^{f/f} and in Kat5^{Δ/Δ} hearts, whereas cells exhibiting these markers were significantly reduced in the remote zone of infarcted Kat5^{Δ/Δ} hearts. Although conditions employed for these assessments precluded co-immunostaining to identify the cell-types undergoing apoptosis, these data suggest the possibility that CM apoptosis, which is increased in the remote zone during post-infarction remodeling,³⁷⁻⁴⁰ is inhibited by depleting Tip60, consistent with its pro-apoptotic function.^{10,41}

In summary, the findings described in FIGS. 26-28 suggest that observations of preserved cardiac function and muscle mass in Tip60-depleted hearts (FIGS. 24-25) may reflect de novo CM generation, as well as diminished cell death. The possibility that CM hypertrophy contributes to preserved function is deemed unlikely, since wheat germ agglutinin (WGA) staining indicated that Tip60 depletion causes a trend toward diminished, rather than increased, CM size in infarcted hearts (FIG. 34).

### Benefits of Tip60 Depletion are not Caused by Cre Recombinase

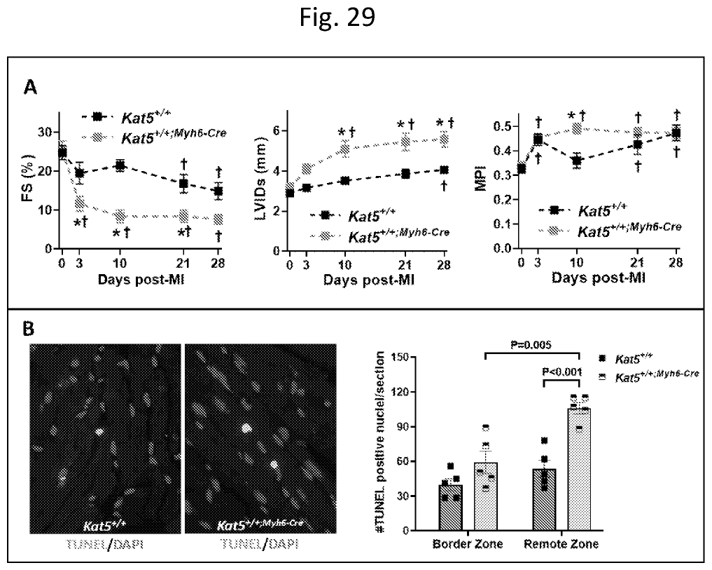

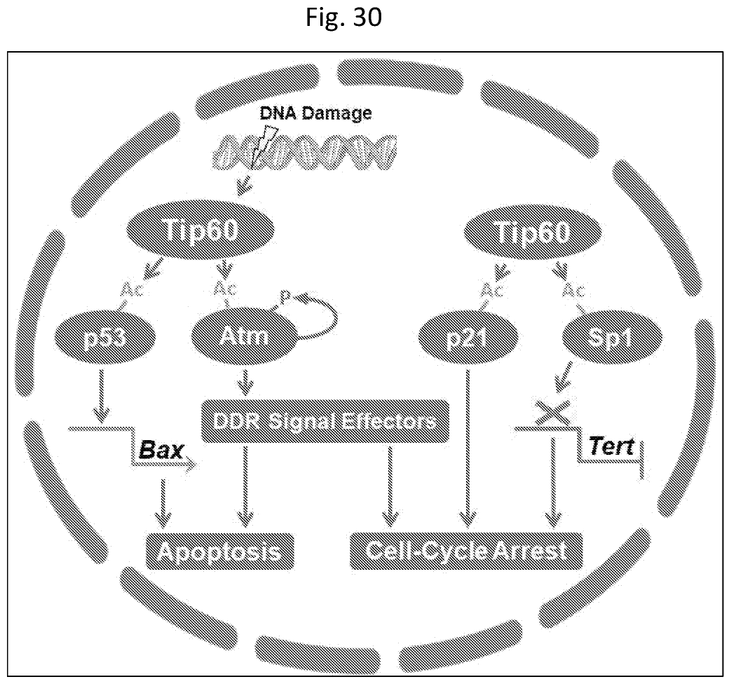

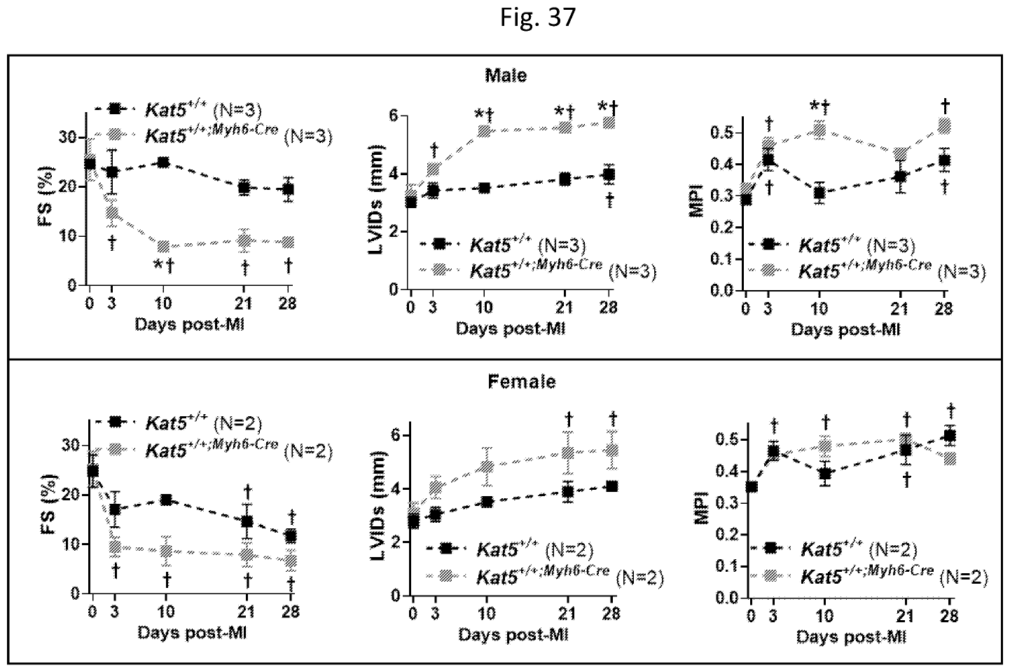

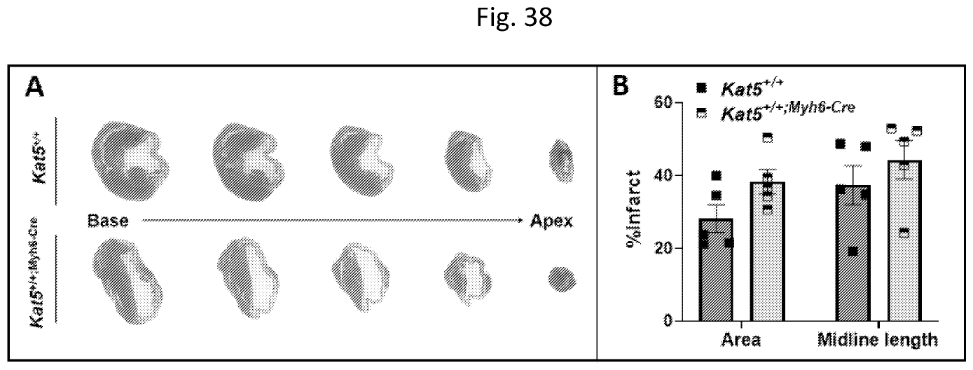

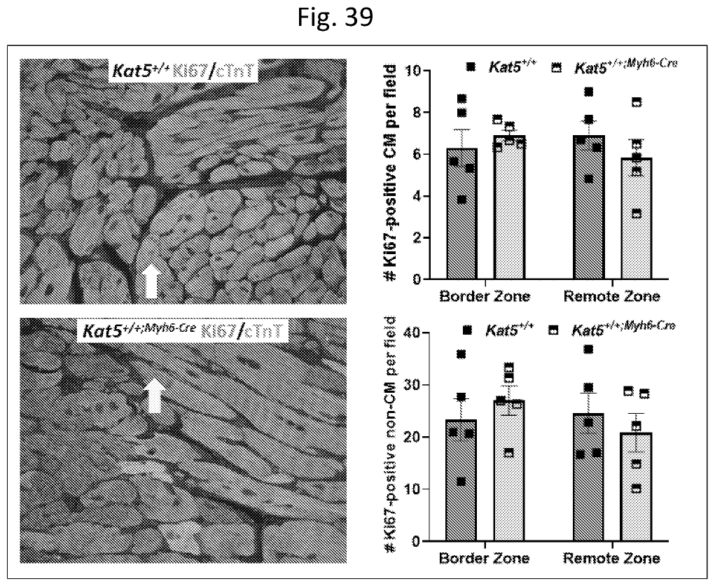

Several laboratories have reported cardiac dysfunction and induction of apoptosis following expression of the Myh6-driven merCremer-recombinase transgene used in this study.⁴²⁻⁴⁵ Although we are aware of no reports of beneficial myocardial function or cellular responses due to Cre-recombinase activation in CMs, it was important to examine this possibility. Hence, control experiments comparing infarcted wild-type Kat5^{+/+} and Kat5^{+/+;Myh6-merCremer} mice injected with tamoxifen 3 days post-MI were performed to assess whether Cre-recombinase caused beneficial effects in the absence of Tip60 depletion. Echocardiography revealed that Cre-recombinase did not improve function, instead causing greater dysfunction at all timepoints (FIG. 29A, FIG. 37, Table 8). Moreover, Cre-recombinase alone had no effect on scarring (FIG. 38) or on CM cell cycle activation (FIG. 39). Finally, instead of reducing apoptosis in the remote zone as in the instance of Tip60 depletion (FIG. 28), the presence of Cre-recombinase alone was associated with increased numbers of TUNEL-positive cells in the remote zone (FIG. 29B). These findings suggest that although Cre recombinase may impair post-MI recovery; these pathogenic effects were overcome in the background of Tip60 depletion.

TABLE 8
Comparison of Echocardiography Assessment of Infarcted Wild-type (Kat5^{+/+}) and Kat5^{+/+;} ^{Myh6-merCremer} Mice.

| | Baseline | | 3 days-post-MI | | 10 days-post-MI | |
|---|---|---|---|---|---|---|
| | Kat5^{+/+} | Kat5^{+/+;} ^{Myh6-Cre} | Kat5^{+/+} | Kat5^{+/+;} ^{Myh6-Cre} | Kat5^{+/+} | Kat5^{+/+;} ^{Myh6-Cre} |
| | N = 5 | N = 5 | N = 5 | N = 5 | N = 5 | N = 5 |
| LVAWd | 0.62 ± 0.03 | 0.62 ± 0.03 | 0.79 ± 0.02† | 0.70 ± 0.07 | 0.68 ± 0.02 | 0.57 ± 0.03 |
| (mm) | | | | | | |
| LVAWs | 0.82 ± 0.03 | 0.85 ± 0.03 | 0.92 ± 0.03 | 0.78 ± 0.08 | 0.85 ± 0.03 | 0.64 ± 0.04*† |
| (mm) | | | | | | |
| LVIDd | 3.83 ± 0.14 | 4.22 ± 0.20 | 3.94 ± 0.19 | 4.64 ± 0.24 | 4.47 ± 0.11 | 5.54 ± 0.37*† |
| (mm) | | | | | | |
| LVIDs | 2.89 ± 0.16 | 3.16 ± 0.24 | 3.16 ± 0.17 | 4.10 ± 0.24 | 3.51 ± 0.06 | 5.09 ± 0.42*† |
| (mm) | | | | | | |
| LVPWd | 0.60 ± 0.02 | 0.62 ± 0.03 | 0.73 ± 0.40 | 0.71 ± 0.07 | 0.68 ± 0.03 | 0.28 ± 0.05 |
| (mm) | | | | | | |
| LVPWs | 0.82 ± 0.03 | 0.85 ± 0.02 | 0.91 ± 0.02 | 0.82 ± 0.08 | 0.91 ± 0.01 | 0.66 ± 0.07*† |
| (mm) | | | | | | |
| HR | 370 ± 10 | 374 ± 11 | 439 ± 4† | 418 ± 13 | 385 ± 28 | 429 ± 2 |
| (bpm) | | | | | | |
| FS | 24.7 ± 1.8 | 25.4 ± 2.3 | 19.5 ± 2.8 | 11.6 ± 1.9*† | 21.4 ± 1.5 | 8.4 ± 1.6*† |
| (%) | | | | | | |
| MPI | 0.33 ± 0.02 | 0.34 ± 0.01 | 0.45 ± 0.02† | 0.46 ± 0.01† | 0.36 ± 0.03 | 0.49 ± 0.02*† |
| LV | 61.4 ± 6.6 | 74.2 ± 6.6 | 77.4 ± 5.3 | 92.2 ± 12.9 | 91.5 ± 5.3 | 110.5 ± 13.0† |
| mass (mg) | | | | | | |
| | | | 21 days-post-MI | | 28 days-post-MI | |
| | | | Kat5^{+/+} | Kat5^{+/+;} ^{Myh6-Cre} | Kat5^{+/+} | Kat5^{+/+;} ^{Myh6-Cre} |
| | | | N = 5 | N = 5 | N = 5 | N = 5 |
| | | LVAWd | 0.63 ± 0.05 | 0.55 ± 0.02 | 0.60 ± 0.04 | 0.53 ± 0.03 |
| | | (mm) | | | | |
| | | LVAWs | 0.73 ± 0.07 | 0.64 ± 0.04† | 0.70 ± 0.05 | 0.61 ± 0.03† |
| | | (mm) | | | | |
| | | LVIDd | 4.63 ± 0.19 | 5.92 ± 0.39*† | 4.76 ± 0.13† | 6.02 ± 0.37*† |
| | | (mm) | | | | |
| | | LVIDs | 3.86 ± 0.22 | 5.45 ± 0.43*† | 4.05 ± 0.15† | 5.58 ± 0.39*† |
| | | (mm) | | | | |
| | | LVPWd | 0.62 ± 0.03 | 0.59 ± 0.04 | 0.64 ± 0.03 | 0.56 ± 0.06 |
| | | (mm) | | | | |
| | | LVPWs | 0.77 ± 0.04 | 0.65 ± 0.06† | 0.82 ± 0.04 | 0.68 ± 0.08 |
| | | (mm) | | | | |
| | | HR | 364 ± 22 | 425 ± 30 | 374 ± 14 | 416 ± 20 |
| | | (bpm) | | | | |
| | | FS | 16.8 ± 2.4† | 8.4 ± 1.5*† | 14.9 ± 2.2† | 7.6 ± 1.3† |
| | | (%) | | | | |
| | | MPI | 0.43 ± 0.04† | 0.48 ± 0.02† | 0.47 ± 0.03† | 0.47 ± 0.02† |
| | | LV | 87.2 ± 6.3 | 121.1 ± 11.5*† | 90.9 ± 5.2 | 118.8 ± 10.1† |
| | | mass (mg) | | | | |

*P < 0.05 vs. Kat5^{+/+} and
†P < 0.05 vs. baseline (Day 0).

### DISCUSSION

The experimental goal of this study was to test the hypothesis that Tip60, a tumor suppressor protein known to inhibit the cell cycle in cultured cells, maintains CMs in a state of proliferative senescence and dormant apoptotic potential, preventing regeneration of the myocardium after injury. Observations reported here that Tip60 depletion after MI maintains cardiac function, reduces scar formation, promotes CM cell cycle activation, and reduces apoptosis are consistent with this hypothesis.

### Reliability of the Tip60 Depletion Model

We recently reported that depletion of Tip60 from CMs using a constitutively active Myh6-Cre transgene,²⁷ which begins to be expressed at late embryonic stages of development, results in lethality due to CM fallout by three months of age.²⁸ Based on our earlier finding that global Tip60 depletion causes early embryolethality,²³ this suggested that CM death was caused by exhaustive Tip60 depletion. Therefore the experimental scheme employed here (FIG. 24A) was designed to induce only temporal reduction of Tip60 in CMs by conditionally activating the product of the Myh6-driven merCremer recombinase transgene.³¹ No untoward effects of Tip60 depletion were observed during the 28-day period following tamoxifen injection into either non-injured (FIG. 31, Table 6) or injured (FIGS. 24-25; Table 7) mice; non-injured mice from which Tip60 was depleted survived for up to five months (FIG. 31B). Moreover, assessments to control for possible off-target effects of Cre-recombinase (FIG. 29, FIGS. 37-39; Table 8), which causes early transient cellular effects to be reported elsewhere, indicated that the beneficial effects observed at 28 days post-MI are exclusively due to Tip60 depletion; hence, the persistent maintenance of cardiac function in infarcted Tip60-depleted hearts throughout the 28-day experimental period (FIG. 24B) occurred despite deleterious effects of Cre-recombinase (FIG. 29). Thus the results reported here are caused by depletion of Tip60, reliably informing Tip60's function in the myocardium.

Is Preservation of Cardiac Function by Tip60 Depletion Due to Regeneration and/or Preservation of CMs?

Periodic echocardiographic evaluation of infarcted mice from which Tip60 was depleted beginning on day 3 post-MI until 28 days post-MI revealed that cardiac function was normalized at 10 days, a condition that was sustained until termination of the experiment at 28 days post-MI (FIG. 24B; Table 7). While the complete absence of dysfunction at rest may in part reflect recovery from relatively small infarctions, it is remarkable that few previously reported interventions produced a similar extent of functional preservation. Among these, hearts engineered to enable Cre-mediated depletion of the Hippo pathway component Salvador similarly exhibited full functional recovery and scar resolution nine weeks after MI. Incredibly, this occurred even though the onset of Salvador depletion was not commenced until 21 days after induction of MI.³² It will therefore be interesting to ascertain whether further delaying the timing of Tip60 depletion after MI confers functional improvement.

Preservation of function at 28 days post-MI was accompanied by significantly diminished myocardial scarring (FIG. 25), concomitant with increased numbers of CMs exhibiting cell cycle activation (FIG. 26) as revealed by monitoring three markers of this process. Activation of the CM cell cycle is consistent with our previous observation of cell cycle activation in CMs of hypertrophied Kat5^{+/-} heterozygous hearts.²⁵ Findings in the present investigation were accompanied by the presence of groups of SMA-positive CMs in the infarct border zone of hearts containing Tip60-depleted CMs (FIG. 27); SMA,^{35, 36} along with other genes that are transiently expressed during heart development in the early embryo, has emerged as a marker for the de-differentiation of CMs occurring prefatory to renewed CM proliferation in the diseased adult myocardium.³⁴ Hence, the aggregate of these observations suggests that Tip60 depletion mediates cardiac regeneration via the de novo differentiation of CMs, in a fashion reminiscent of the mechanism occurring during heart development in the early embryo. The observation that non-CMs exhibited a similar extent of cell cycle activation (FIG. 36) was surprising because Myh6-Cre-mediated depletion of Tip60 is theoretically confined to CMs; it is speculated that this may reflect pro-proliferative paracrine signaling elicited by Tip60-depleted CMs. Although these data cannot discern whether Tip60 depletion promotes cardiac regeneration via cell cycle-activated hypertrophy or by advancement to mononuclear diploid daughter cells (i.e. complete progression through cytokinesis), our evaluation of WGA-stained transverse sections of myocardium revealed that CM size was certainly not increased, and may have been decreased, by Tip60 depletion (FIG. 34); we are currently attempting to distinguish these possibilities by employing the rigorous approach of enumerating numbers of newly generated mononuclear diploid cardiomyocytes (MNDCMs⁴⁶) in infarcted Tip60-depleted hearts.

As shown in FIG. 28, preservation of function also correlated with significantly reduced numbers of apoptotic cells in the remote zone of Tip60-depleted hearts at 28 days post-MI. As in the instance of increased cell cycle activation, this finding is consistent with the diminished numbers of apoptotic cells we previously observed in the myocardium of hypertrophied Kat5^{+/-} heterozygous hearts,²⁵ as well as with well-documented findings that Tip60 is pro-apoptotic.^{10, 11, 47-49} Although we were unable to specify identity of the apoptotic cells, this finding suggests that Tip60 depletion may mitigate CM losses occurring in the remote zone during pathogenic post-infarction remodeling of the left ventricle.^{40, 50, 51} This possibility warrants further investigation.

### Implications

As recently suggested,² progress in the field of cardiac regeneration would be advanced by the identification, and ability to release the effects of, inhibitory factors that have evolved to maintain CMs in their profound state of proliferative senescence. Of course, regenerative approaches based on the relief of inhibitory factors would also mandate interventions to regulate CM proliferation, once unleased, in order to prevent rhabdomyosarcoma formation⁵² and/or mitotic catastrophe,^{5, 53} as observed following Gsk-3 depletion; regarding the latter it is curious that Gsk-3 has been shown to activate Tip60.^{47, 54, 55} Several inhibitory proteins, mostly tumor suppressors, have been identified that when deleted result in activation of the CM cell cycle; these include Gsk-3,⁵⁶ Retinoblastomal,^{3, 57} Meis1⁴ and Meis2,⁵⁷ and Hippo pathway components.^{32, 58} Improved regenerative efficacy via simultaneous depletion of two of these inhibitors was recently reported.⁵⁷ We are now investigating the intriguing possibility that the regenerative efficacy of Tip60 depletion in CMs is mediated by down-regulating one or more of its documented inhibitory pathways depicted in FIG. 30. Among these, the possibility that Tip60 activates Atm to initiate the DDR, which culminates in proliferative senescence, is consistent with recent reports showing that Atm de-activation promotes CM proliferation in the adult heart,³⁰ and that Atm depletion alleviates effects of DDR-induced heart failure.⁵⁹ The findings reported here justify inclusion of Tip60 to the list of potential cardiac therapeutic targets.

### REFERENCES

1. Sadek H and Olson E N. Toward the Goal of Human Heart Regeneration. Cell Stem Cell. 2020; 26:7-16.
2. Galdos F X, Guo Y, Paige S L, VanDusen N J, Wu S M and Pu W T. Cardiac Regeneration: Lessons From Development. Circ Res. 2017; 120:941-959.
3. Sdek P, Zhao P, Wang Y, Huang C J, Ko C Y, Butler P C, Weiss J N and Maclellan W R. Rb and p130 control cell cycle gene silencing to maintain the postmitotic phenotype in cardiac myocytes. J Cell Biol. 2011; 194:407-23.
4. Mahmoud A I, Kocabas F, Muralidhar S A, Kimura W, Koura A S, Thet S, Porrello E R and Sadek H A. Meis1 regulates postnatal cardiomyocyte cell cycle arrest. Nature. 2013; 497:249-253.
5. Zhou J, Ahmad F, Parikh S, Hoffman N E, Rajan S, Verma V K, Song J, Yuan A, Shanmughapriya S, Guo Y, Gao E, Koch W, Woodgett J R, Madesh M, Kishore R, Lal H and Force T. Loss of Adult Cardiac Myocyte GSK-3 Leads to Mitotic Catastrophe Resulting in Fatal Dilated Cardiomyopathy. Circ Res. 2016; 118:1208-22.
6. Heallen T, Morikawa Y, Leach J, Tao G, Willerson J T, Johnson R L and Martin J F. Hippo signaling impedes adult heart regeneration. Development. 2013; 140:4683-90.
7. Sun Y, Jiang X and Price B D. Tip60: connecting chromatin to DNA damage signaling. Cell Cycle. 2010; 9:930-6.
8. Sun Y, Jiang X, Xu Y, Ayrapetov M K, Moreau L A, Whetstine J R and Price B D. Histone H3 methylation links DNA damage detection to activation of the tumour suppressor Tip60. Nat Cell Biol. 2009; 11:1376-82.
9. Sun Y, Jiang X, Chen S, Fernandes N and Price B D. A role for the Tip60 histone acetyltransferase in the acetylation and activation of ATM. Proc Natl Acad Sci USA. 2005; 102:13182-7.
10. Sykes S M, Mellert H S, Holbert M A, Li K, Marmorstein R, Lane W S and McMahon S B. Acetylation of the p53 DNA-binding domain regulates apoptosis induction. Mol Cell. 2006; 24:841-51.
11. Tang Y, Luo J, Zhang W and Gu W. Tip60-dependent acetylation of p53 modulates the decision between cell-cycle arrest and apoptosis. Mol Cell. 2006; 24:827-39.
12. Squatrito M, Gorrini C and Amati B. Tip60 in DNA damage response and growth control: many tricks in one HAT. Trends Cell Biol. 2006; 16:433-42.
13. Muckova K, Duffield J S, Held K D, Bonventre J V and Sheridan A M. cPLA2-interacting protein, PLIP, causes apoptosis and decreases G₁ phase in mesangial cells. Am J Physiol Renal Physiol. 2006; 290:F70-9.
14. Lee M S, Seo J, Choi D Y, Lee E W, Ko A, Ha N C, Yoon J B, Lee H W, Kim K P and Song J. Stabilization of p21 (Cip1/WAF1) following Tip60-dependent acetylation is required for p21-mediated DNA damage response. Cell death and differentiation. 2013; 20:620-9.
15. Rajagopalan D, Pandey A K, Xiuzhen M C, Lee K K, Hora S, Zhang Y, Chua B H, Kwok H S, Bhatia S S, Deng L W, Tenen D G, Kappei D and Jha S. TIP60 represses telomerase expression by inhibiting Sp1 binding to the TERT promoter. PLoS pathogens. 2017; 13:e1006681.
16. Bose A, Sudevan S, Rao V J, Shima H, Trivedi A K, Igarashi K and Kundu T K. Haploinsufficient tumor suppressor Tip60 negatively regulates oncogenic Aurora B kinase. J Biosci. 2019; 44.
17. Gorrini C, Squatrito M, Luise C, Syed N, Perna D, Wark L, Martinato F, Sardella D, Verrecchia A, Bennett S, Confalonieri S, Cesaroni M, Marchesi F, Gasco M, Scanziani E, Capra M, Mai S, Nuciforo P, Crook T, Lough J and Amati B. Tip60 is a haplo-insufficient tumour suppressor required for an oncogene-induced DNA damage response. Nature. 2007; 448:1063-7.
18. Gao C, Bourke E, Scobie M, Famme M A, Koolmeister T, Helleday T, Eriksson L A, Lowndes N F and Brown J A. Rational design and validation of a Tip60 histone acetyltransferase inhibitor. Sci Rep. 2014; 4:5372.
19. McGuire A, Casey M C, Shalaby A, Kalinina O, Curran C, Webber M, Callagy G, Holian E, Bourke E, Kerin M J and Brown J A L. Quantifying Tip60 (Kat5) stratifies breast cancer. Sci Rep. 2019; 9:3819.
20. Coffey K, Blackburn T J, Cook S, Golding B T, Griffin R J, Hardcastle I R, Hewitt L, Huberman K, McNeill H V, Newell D R, Roche C, Ryan-Munden C A, Watson A and Robson C N. Characterisation of a Tip60 specific inhibitor, NU9056, in prostate cancer. PLoS One. 2012; 7:e45539.
21. Halkidou K, Gnanapragasam V J, Mehta P B, Logan I R, Brady M E, Cook S, Leung H Y, Neal D E and Robson C N. Expression of Tip60, an androgen receptor coactivator, and its role in prostate cancer development. Oncogene. 2003; 22:2466-77.
22. Lough J W. Transient expression of TIP60 protein during early chick heart development. Dev Dyn. 2002; 223:419-25.
23. Hu Y, Fisher J B, Koprowski S, McAllister D, Kim M S and Lough J. Homozygous disruption of the Tip60 gene causes early embryonic lethality. Dev Dyn. 2009; 238:2912-21.
24. Xiao G, Mao S, Baumgarten G, Serrano J, Jordan M C, Roos K P, Fishbein M C and MacLellan W R. Inducible activation of c-Myc in adult myocardium in vivo provokes cardiac myocyte hypertrophy and reactivation of DNA synthesis. Circ Res. 2001; 89:1122-9.
25. Fisher J B, Kim M S, Blinka S, Ge Z D, Wan T, Duris C, Christian D, Twaroski K, North P, Auchampach J and Lough J. Stress-induced cell-cycle activation in Tip60 haploinsufficient adult cardiomyocytes. PLoS One. 2012; 7:e31569.
26. Gogna R, Madan E, Khan M, Pati U and Kuppusamy P. p53's choice of myocardial death or survival: Oxygen protects infarct myocardium by recruiting p53 on NOS3 promoter through regulation of p53-Lys(118) acetylation. EMBO Mol Med. 2013; 5:1662-83.
27. Oka T, Maillet M, Watt A J, Schwartz R J, Aronow B J, Duncan S A and Molkentin J D. Cardiac-specific deletion of Gata4 reveals its requirement for hypertrophy, compensation, and myocyte viability. Circ Res. 2006; 98:837-45.
28. Fisher J B, Horst A, Wan T, Kim M S, Auchampach J and Lough J. Depletion of Tip60 from In Vivo Cardiomyocytes Increases Myocyte Density, Followed by Cardiac Dysfunction, Myocyte Fallout and Lethality. PLoS One. 2016; 11:e0164855.
29. Puente B N, Kimura W, Muralidhar S A, Moon J, Amatruda J F, Phelps K L, Grinsfelder D, Rothermel B A, Chen R, Garcia J A, Santos C X, Thet S, Mori E, Kinter M T, Rindler P M, Zacchigna S, Mukherjee S, Chen D J, Mahmoud A I, Giacca M, Rabinovitch P S, Aroumougame A, Shah A M, Szweda L I and Sadek H A. The oxygen-rich postnatal environment induces cardiomyocyte cell-cycle arrest through DNA damage response. Cell. 2014; 157:565-79.
30. Nakada Y, Canseco D C, Thet S, Abdisalaam S, Asaithamby A, Santos C X, Shah A M, Zhang H, Faber J E, Kinter M T, Szweda L I, Xing C, Hu Z, Deberardinis R J, Schiattarella G, Hill J A, Oz O, Lu Z, Zhang C C, Kimura W and Sadek H A. Hypoxia induces heart regeneration in adult mice. Nature. 2017; 541:222-227.
31. Sohal D S, Nghiem M, Crackower M A, Witt S A, Kimball T R, Tymitz K M, Penninger J M and Molkentin J D. Temporally regulated and tissue-specific gene manipulations in the adult and embryonic heart using a tamoxifen-inducible Cre protein. Circ Res. 2001; 89:20-5.
32. Leach J P, Heallen T, Zhang M, Rahmani M, Morikawa Y, Hill M C, Segura A, Willerson J T and Martin J F. Hippo pathway deficiency reverses systolic heart failure after infarction. Nature. 2017; 550:260-264.
33. Pinto A R, Ilinykh A, Ivey M J, Kuwabara J T, D'Antoni M L, Debuque R, Chandran A, Wang L, Arora K, Rosenthal N A and Tallquist M D. Revisiting Cardiac Cellular Composition. Circ Res. 2016; 118:400-9.
34. Kubin T, Poling J, Kostin S, Gajawada P, Hein S, Rees W, Wietelmann A, Tanaka M, Lorchner H, Schimanski S, Szibor M, Warnecke H and Braun T. Oncostatin M is a major mediator of cardiomyocyte dedifferentiation and remodeling. Cell Stem Cell. 2011; 9:420-32.
35. Sugi Y and Lough J. Onset of expression and regional deposition of alpha-smooth and sarcomeric actin during avian heart development. Dev Dyn. 1992; 193:116-24.
36. Ruzicka D L and Schwartz R J. Sequential activation of alpha-actin genes during avian cardiogenesis: vascular smooth muscle alpha-actin gene transcripts mark the onset of cardiomyocyte differentiation. J Cell Biol. 1988; 107:2575-86.
37. Schwarz K, Simonis G, Yu X, Wiedemann S and Strasser R H. Apoptosis at a distance: remote activation of caspase-3 occurs early after myocardial infarction. Mol Cell Biochem. 2006; 281:45-54.
38. Joki Y, Ohashi K, Yuasa D, Shibata R, Ito M, Matsuo K, Kambara T, Uemura Y, Hayakawa S, Hiramatsu-Ito M, Kanemura N, Ogawa H, Daida H, Murohara T and Ouchi N. FGF21 attenuates pathological myocardial remodeling following myocardial infarction through the adiponectin-dependent mechanism. Biochem Biophys Res Commun. 2015; 459:124-30.
39. Dassanayaka S, Brittian K R, Jurkovic A, Higgins L A, Audam T N, Long B W, Harrison L T, Militello G, Riggs D W, Chitre M G, Uchida S, Muthusamy S, Gumpert A M and Jones S P. E2f1 deletion attenuates infarct-induced ventricular remodeling without affecting O-GlcNAcylation. Basic Res Cardiol. 2019; 114:28.
40. Wang Y, Chen Y, Yan Y, Li X, Chen G, He N, Shen S, Chen G, Zhang C, Liao W, Liao Y and Bin J. Loss of CEACAM1, a Tumor-Associated Factor, Attenuates Post-infarction Cardiac Remodeling by Inhibiting Apoptosis. Sci Rep. 2016; 6:21972.
41. Tang Y, Zhao W, Chen Y, Zhao Y and Gu W. Acetylation is indispensable for p53 activation. Cell. 2008; 133:612-26.
42. Koitabashi N, Bedja D, Zaiman A L, Pinto Y M, Zhang M, Gabrielson K L, Takimoto E and Kass D A. Avoidance of transient cardiomyopathy in cardiomyocyte-targeted tamoxifen-induced MerCreMer gene deletion models. Circ Res. 2009; 105:12-5.
43. Hall M E, Smith G, Hall J E and Stec D E. Systolic dysfunction in cardiac-specific ligand-inducible MerCreMer transgenic mice. Am J Physiol Heart Circ Physiol. 2011; 301:H253-60.
44. Bersell K, Choudhury S, Mollova M, Polizzotti B D, Ganapathy B, Walsh S, Wadugu B, Arab S and Kuhn B. Moderate and high amounts of tamoxifen in alphaMHC-MerCreMer mice induce a DNA damage response, leading to heart failure and death. Dis Model Mech. 2013; 6:1459-69.
45. Lexow J, Poggioli T, Sarathchandra P, Santini M P and Rosenthal N. Cardiac fibrosis in mice expressing an inducible myocardial-specific Cre driver. Dis Model Mech. 2013; 6:1470-6.
46. Patterson M, Barske L, Van Handel B, Rau C D, Gan P, Sharma A, Parikh S, Denholtz M, Huang Y, Yamaguchi Y, Shen H, Allayee H, Crump J G, Force T I, Lien C L, Makita T, Lusis A J, Kumar S R and Sucov H M. Frequency of mononuclear diploid cardiomyocytes underlies natural variation in heart regeneration. Nat Genet. 2017; 49:1346-1353.
47. Charvet C, Wissler M, Brauns-Schubert P, Wang S J, Tang Y, Sigloch F C, Mellert H, Brandenburg M, Lindner S E, Breit B, Green D R, McMahon S B, Borner C, Gu W and Maurer U. Phosphorylation of Tip60 by GSK-3 determines the induction of PUMA and apoptosis by p53. Mol Cell. 2011; 42:584-96.
48. Xu Y, Liao R, Li N, Xiang R and Sun P. Phosphorylation of Tip60 by p38alpha regulates p53-mediated PUMA induction and apoptosis in response to DNA damage. Oncotarget. 2014; 5:12555-72.
49. Ikura T, Ogryzko V V, Grigoriev M, Groisman R, Wang J, Horikoshi M, Scully R, Qin J and Nakatani Y. Involvement of the TIP60 histone acetylase complex in DNA repair and apoptosis. Cell. 2000; 102:463-73.
50. Dorn G W, 2nd. Apoptotic and non-apoptotic programmed cardiomyocyte death in ventricular remodelling. Cardiovasc Res. 2009; 81:465-73.
51. Dorn G W, 2nd. Novel pharmacotherapies to abrogate postinfarction ventricular remodeling. Nature reviews Cardiology. 2009; 6:283-91.
52. Gabisonia K, Prosdocimo G, Aquaro G D, Carlucci L, Zentilin L, Secco I, Ali H, Braga L, Gorgodze N, Bernini F, Burchielli S, Collesi C, Zandona L, Sinagra G, Piacenti M, Zacchigna S, Bussani R, Recchia F A and Giacca M. MicroRNA therapy stimulates uncontrolled cardiac repair after myocardial infarction in pigs. Nature. 2019; 569:418-422.
53. Singh A P, Umbarkar P, Guo Y, Force T, Gupte M and Lal H. Inhibition of GSK-3 to induce cardiomyocyte proliferation: a recipe for in situ cardiac regeneration. Cardiovasc Res. 2019; 115:20-30.
54. Cheng X, Ma X, Zhu Q, Song D, Ding X, Li L, Jiang X, Wang X, Tian R, Su H, Shen Z, Chen S, Liu T, Gong W, Liu W and Sun Q. Pacer Is a Mediator of mTORC1 and GSK3-TIP60 Signaling in Regulation of Autophagosome Maturation and Lipid Metabolism. Mol Cell. 2019; 73:788-802 e7.
55. Lin S Y, Li T Y, Liu Q, Zhang C, Li X, Chen Y, Zhang S M, Lian G, Liu Q, Ruan K, Wang Z, Zhang C S, Chien K Y, Wu J, Li Q, Han J and Lin S C. GSK3-TIP60-ULK1 signaling pathway links growth factor deprivation to autophagy. Science. 2012; 336:477-81.
56. Kerkela R, Kockeritz L, Macaulay K, Zhou J, Doble B W, Beahm C, Greytak S, Woulfe K, Trivedi C M, Woodgett J R, Epstein J A, Force T and Huggins G S. Deletion of GSK-3beta in mice leads to hypertrophic cardiomyopathy secondary to cardiomyoblast hyperproliferation. J Clin Invest. 2008; 118:3609-18.
57. Alam P, Haile B, Arif M, Pandey R, Rokvic M, Nieman M, Maliken B D, Paul A, Wang Y G, Sadayappan S, Ahmed R P H and Kanisicak O. Inhibition of Senescence-Associated Genes Rb1 and Meis2 in Adult Cardiomyocytes Results in Cell Cycle Reentry and Cardiac Repair Post-Myocardial Infarction. J Am Heart Assoc. 2019; 8:e012089.
58. Heallen T, Zhang M, Wang J, Bonilla-Claudio M, Klysik E, Johnson R L and Martin J F. Hippo pathway inhibits Wnt signaling to restrain cardiomyocyte proliferation and heart size. Science. 2011; 332:458-61.
59. Higo T, Naito A T, Sumida T, Shibamoto M, Okada K, Nomura S, Nakagawa A, Yamaguchi T, Sakai T, Hashimoto A, Kuramoto Y, Ito M, Hikoso S, Akazawa H, Lee J K, Shiojima I, McKinnon P J, Sakata Y and Komuro I. DNA single-strand break-induced DNA damage response causes heart failure. Nature communications. 2017; 8:15104.
60. Oh H, Wang S C, Prahash A, Sano M, Moravec C S, Taffet G E, Michael L H, Youker K A, Entman M L and Schneider M D. Telomere attrition and Chk2 activation in human heart failure. Proc Natl Acad Sci USA. 2003; 100:5378-83.

### Example 3: Tip60 Inhibitor as a Cardioprotector

In this Example, we test whether a small molecule organic drug, NU9056, which specifically inhibits Tip60 acetyltransferase activity, is cardioprotective. NU9056's effects will be screened in a neonatal CM cell culture model, and in vivo experiments will be performed in adult mouse MI models. Not to be bound by theory, but we believe drugs that inhibit Tip60's acetyltransferase (HAT) domain, thereby inhibiting site-specific acetylation of pAtm, may temporarily permit CM proliferation to regenerate the infarcted myocardium. Inactivation of Tip60's HAT domain may also prevent acetylation of proteins that drive p21 transcription, thereby relieving cell cycle blockade. Because Tip60 also acetylates p53, which downstream regulates expression of Bax, inhibition of Tip60 may have the dual benefit of inhibiting apoptosis while promoting CM regeneration.

Recently, small molecule drugs that specifically inactivate Tip60's HAT domain, including NU9056, have become commercially available (Tocris; Minneapolis, Minn.; #4903). NU9056 inhibits Tip60's HAT domain relative to the acetylase domains of other HAT proteins (i.e. IC50 values for Tip60, p300, pCAF & GCN5 are respectively <2, 60, 36 & >100 μM).

A wide range of NU9056 concentrations (0.03-100 μM NU9056) will be applied to cultures of homogeneously dispersed wild-type rat neonatal CMs for 24 hours. The cells will then be assessed for cell proliferation by direct cell counting and [3H]-thymidine incorporation as well as by immunostaining for Ki67, BrdU and pH3. Apoptosis will also be evaluated, by TUNEL labeling and by staining of caspase-3. All results will be confirmed using a minimum of three independent determinations, and statistical evaluation will be performed using ANOVA or a Student's t-test, as appropriate.

We expect that low concentrations of NU9056 (0.1-3 μM) will inhibit apoptosis and promote CM proliferation.

Two MI models, ischemia/reperfusion (I/R) and permanent occlusion will be used to test whether NU9056 reduces infarct size and promotes CM regeneration. Infarcted mice will be treated once daily for seven days with NU9056 (or vehicle), with the first dose administered 1 day after coronary occlusion. In the I/R model, mice will be treated daily for 7 days with the first dose administered at the onset of reperfusion and cardioprotection will be evaluated by monitoring plasma cTnI levels, infarct size, and extent of contractile dysfunction/ventricular remodeling (i.e., using echocardiography). In the permanent ligation model, effects will be evaluated by scar size (trichrome staining) and contractile dysfunction/ventricular remodeling (echocardiography). After establishing that NU9056 has a positive effect, molecular/cellular studies will be conducted to assess effects on apoptosis, and CM proliferation. Initial doses to be tested, i.e. 4, 40, and 400 mg/mouse/day, are based on desired peak blood levels of 1, 10 and 100 μM, assuming that the drug is evenly distributed within total body water (˜18 ml/25 g male mouse). Dosages, administration frequency and treatment duration will be refined based on accumulating evidence.

1. The method of inducing proliferation of adult cardiomyocytes (CMs), the method comprising: transiently contacting the adult CMs with an effective amount of a Tip60 inhibitor to induce proliferation of cardiomyocytes.

2. The method of claim 1, wherein adult cardiomyocytes are within a patient, and where the contacting step comprises administering to the patient the Tip60 inhibitor to induce in vivo proliferation of cardiomyocytes.

3. The method of claim 2, wherein the patient has suffered from cardiac injury.

4. The method of claim 2, wherein the patient has suffered from myocardial infarction.

5. The method of claim 3, wherein the Tip60 inhibitor is administered within 5 days of the cardiac injury or myocardial infarction.

6. The method of claim 3, wherein the Tip60 inhibitor is administered for a sufficient time to induce in vivo proliferation of the patient's cardiomyocytes.

7. A method of regenerating heart tissue within a patient in need thereof, the method comprising administering an effective amount of a Tip60 inhibitor to transiently induce proliferation and regeneration of heart tissue.

8. A method of treating a subject with cardiac injury, the method comprising: administering a therapeutically effective amount of a Tip60 inhibitor to treat the cardiac injury.

9. The method of claim 8, wherein the Tip60 inhibitor is administered transiently.

10. The method of claim 8, wherein the cardiac injury is the result of a myocardial infarction.

11. The method of claim 8, wherein the Tip60 inhibitor is NU9056.

12. The method of claim 2, wherein method further comprises administering a glycogen synthase kinase (GSK) inhibitor to the patient.

13. The method of claim 2, wherein the patient is a human.

14. (canceled)

15. The method of claim 1, wherein the Tip60 inhibitor is NU9056.

16. The method of claim 7, wherein the Tip60 inhibitor is NU9056.

17. The method of claim 7, wherein method further comprises administering a glycogen synthase kinase (GSK) inhibitor to the patient.

18. The method of claim 8, wherein method further comprises administering a glycogen synthase kinase (GSK) inhibitor to the patient
